(19) United States
(12) Patent Application Publication
(10) Pub. No.: US 2025/0130231 A1
(43) Pub. Date: Apr. 24, 2025

(54) COMPOSITIONS AND METHODS FOR DETECTION AND TREATMENT OF CORONAVIRUS INFECTION

(71) Applicants: THE TRANSLATIONAL GENOMICS RESEARCH INSTITUTE, Phoenix, AZ (US); ARIZONA BOARD OF REGENTS ON BEHALF OF NORTHERN ARIZONA UNIVERSITY, Flagstaff, AZ (US)

(72) Inventors: John Altin, Flagstaff, AZ (US); Jason Ladner, Flagstaff, AZ (US)

(21) Appl. No.: 18/988,878

(22) Filed: Dec. 19, 2024

Related U.S. Application Data
(62) Division of application No. 17/395,330, filed on Aug. 5, 2021, now Pat. No. 12,247,978.
(60) Provisional application No. 63/061,344, filed on Aug. 5, 2020.

(51) Int. Cl. G01N 33/569; A61K 31/18; A61K 31/22; A61K 31/40; A61K 31/7052; A61K 39/215; A61P 11/00; C12N 7/00; G01N 33/68

(52) U.S. Cl. CPC G01N 33/56983; A61K 31/18; A61K 31/22; A61K 31/40; A61K 31/7052; A61K 39/215; A61P 11/00; C12N 7/00; G01N 33/6884; C12N 2770/20034; G01N 2333/165; G01N 2800/12; G01N 2800/245; G01N 2800/50

(57) ABSTRACT

The present disclosure includes a multiplexed peptide assay to generate an epitope-resolved view of antibody reactivity across all human coronaviruses (CoVs). PepSeq accurately classifies SARS-CoV-2 exposure status and reveals epitopes across the Spike and Nucleocapsid proteins. Two of these represent recurrent reactivities to conserved, functionally-important sites in the S2 subunit of Spike, regions that we show are also targeted for the endemic CoVs in pre-pandemic controls. At one of these sites, we demonstrate that the SARS-CoV-2 response strongly and recurrently cross-reacts with the endemic virus hCoV-OC43. The disclosed epitope-resolved analysis reveals new CoV targets for the development of diagnostics, vaccines and therapeutics, including a site that may have broad neutralizing potential.

### CROSS REFERENCE TO RELATED APPLICATIONS

This application claims the benefit of U.S. Provisional Application No. 63/061,344, filed on Aug. 5, 2020, the content of which is incorporated herein by reference in its entirety.

### STATEMENT REGARDING FEDERALLY SPONSORED RESEARCH

This invention was made with government support under U24AI152172 and U54MD012388 awarded by the National Institute of Health. The government has certain rights in this invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 155,047 byte ASCII (text) file named “91482.251PAT_Seq Listing_ST25” created on Aug. 5, 2021.

### TECHNICAL FIELD

The present invention is related to compositions and methods for diagnosing, treating, and preventing disease and predicting immune response to disease, and more particularly, is related to compositions and methods for diagnosing, treatment, prevention, and prognosis of coronavirus infections.

### BACKGROUND

Severe acute respiratory syndrome coronavirus 2 (SARS-CoV-2) is a single-stranded RNA virus in the Coronaviridae family that emerged in late 2019 and has caused morbidity, mortality and economic disruption on a global scale with few precedents (Zhu et al., 2020). The Coronaviridae family includes four species/strains that are endemic in the human population and usually associated with mild, self-limiting upper respiratory tract infections: HCoV-229E, HCoV-NL63, HCoV-HKU1 and HCoV-OC43 (Betacoronavirus 1 species). Two other species, MERS-CoV and SARS-CoV, have recently emerged to cause severe disease in humans. Like the other human-infecting coronavirus (CoV) species (Callow et al., 1990; Dijkman et al., 2008), SARS-CoV-2 infection can elicit a robust antibody response in humans (Liu et al., 2020; Ni et al., 2020) and this response represents the major focus of widespread efforts to develop accurate diagnostics, as well as strategies for passive and active immunization against infection (Casadevall and Pirofski, 2020; Thanh Le et al., 2020; Krammer and Simon, 2020). Existing serological assays for SARS-CoV-2 antibody reactivity generally use full-length viral proteins or domains—Spike(S) , Nucleocapsid (N), or the receptor-binding domain (RBD) of S—as antigenic baits, followed by enzyme-linked or fluorescent detection (Krammer and Simon, 2020). These assays provide a single measure of antibody reactivity, which represents a composite signal across many epitopes, and are able to detect viral exposure with a range of accuracies (Deeks et al., 2020; Whitman et al., 2020). Neutralization assays using either native or pseudotyped viruses have also been developed (Nie et al., 2020). It remains to be seen how these different assays will perform as diagnostics or correlates of the protection conferred by infection or vaccination.

Relative to protein-based analyses of the humoral response, epitope-level assays have the potential to add several layers of information. First, although SARS-CoV-2 proteins are generally distinct from other human-infecting Coronaviruses, some regions of strong homology exist (Lu et al., 2020; Zhu et al., 2020), meaning that there is the potential for immune cross-reactivity that can only be resolved at the epitope level. Indeed, it was recently demonstrated that a large fraction of non-exposed individuals have T cell reactivity to SARS-CoV-2 peptides, indicating cross-reactivity with existing responses, possibly those generated against homologous peptides from endemic CoV strains (Grifoni et al., 2020). In the case of antibody responses, cross-reactivity has been described between the more closely related SARS-CoV and SARS-CoV-2 (Lv et al., 2020; Pinto et al., 2020). Epitope-resolved analyses therefore have the potential to identify antigens that may discriminate related CoVs, leading to more specific diagnostic assays. High levels of sequence conservation may also indicate functional essentiality; therefore, by highlighting potentially cross-reactive epitopes in conserved regions of the proteome, epitope-level assays can identify antibodies and targets with therapeutic potential, against which viral escape may be more difficult (Friesen et al., 2014).

A second rationale for generating epitope-resolved views is that antibody recognition of different protein regions can have divergent functional consequences, including neutralization potential. For coronaviruses, antibodies binding the surface-exposed, receptor-binding S protein exhibit the greatest neutralizing potential (Du et al., 2009; Pillay, 2020), but these antibodies can recognize a wide variety of epitopes within the protein, each with the potential for different functional consequences. This likely accounts for the imperfect correlation between the titers of S-binding antibodies and viral neutralization activity across individuals (Robbiani et al., 2020). Due to its interaction with the host entry receptor (the angiotensin converting enzyme 2—ACE2), the RBD of S represents the predominant target of vaccination and monoclonal antibody development strategies, and a growing number of antibodies against this domain have been described (Chi et al., 2020; Hansen et al., 2020; Robbiani et al., 2020; Zost et al., 2020). However, antibodies against epitopes outside of the RBD have also been shown to have neutralizing activity (Chi et al., 2020; Poh et al., 2020): these may act in various ways, including by preventing important protease cleavage events and/or conformational changes required for successful entry into cells. On the other hand, antibodies that recognize epitopes within the N protein, which coats the viral genome and is contained within mature viral particles, likely provide little or no neutralization potential, but may be useful signatures for differentiating vaccine responses from those resulting from natural virus infection, a strategy already used for other viruses (Hoofnagle et al., 1974; Lubroth et al., 1996). In addition to different neutralization potential, it is possible that unfavorable distributions of epitope reactivity can contribute to immunopathology, for example through antibody dependent enhancement (Halstead and O'rourke, 1977; Katzelnick et al., 2017; Khurana et al., 2013), although this phenomenon remains to be demonstrated for SARS-CoV-2 (Eroshenko et al., 2020).

Peptide sub-sequences have been used for decades as probes to detect antibodies recognizing linear epitopes within the full-length proteins from which they are derived (Fleri et al., 2017; Lucchese et al., 2007). Although unable to detect antibodies whose binding depends on elements that are distal in the primary sequence, this strategy has the advantage that it enables the highly-efficient design and synthesis of antigen baits. In its simplest format, peptides can be used individually, for example in separate wells in an ELISA. A recent study used this approach to identify two linear epitopes in S protein that were targeted by neutralizing antibodies in SARS-CoV-2 convalescent donors (Poh et al., 2020). More powerful assays involve sets of peptides that are assayed in multiplex-using either spatial addressing, in the case of peptide arrays (Price et al., 2012), or DNA indexing, in the case of phage display libraries (Larman et al., 2011). Using the latter approach, the highly-multiplexed and epitope-resolved detection of antibodies to viruses has been demonstrated with high sensitivity and specificity (Xu et al., 2015).

### SUMMARY

A need exists for methods of diagnosing, treating, preventing, and determining an immune response to coronavirus infections, including COVID-19 caused by SARS-CoV-2 infection. A high-resolution understanding of the antibody response to SARS-CoV-2 is important for the design of effective diagnostics, vaccines and therapeutics. However, SARS-CoV-2 antibody epitopes remain largely uncharacterized, and it is unknown whether and how the response may cross-react with related viruses.

The present disclosure presents a synthetic biology approach to highly-multiplexed peptide-based serological assays in which libraries of peptide baits—each covalently coupled to a DNA barcode—are synthesized from high-complexity DNA pools using a simple and fully in vitro approach. Library synthesis takes advantage of in vitro transcription and translation, including an intramolecular coupling mediated by puromycin (Kozlov et al., 2012) and the DNA-barcoded peptides can then be used to probe serum antibodies using a high-throughput sequencing read-out. We use this platform to synthesize libraries of overlapping 30mers covering all human coronavirus (CoV) proteomes and assay these against sera from pre-pandemic and SARS-CoV-2 convalescent donors. Our results demonstrate accurate detection of SARS-CoV-2 exposure and reveal multiple immunodominant antibody epitopes, including at least one in which antibody responses cross-react between SARS-CoV-2 and an endemic human CoV.

In some aspects, the present disclosure relates to a method of detecting in a sample the presence of an antibody that binds to a spike protein or a nucleocapsid protein of a severe acute respiratory syndrome-associated coronavirus (SARS-CoV), the method comprising: providing a biological sample from a subject suspected to be infected with a SARS-CoV; contacting the biological sample with a peptide comprising an amino acid sequence having at least 85% sequence identity to an amino acid sequence selected from the group consisting of SEQ ID NOs: 1-4, 275-412, and 421-423; and detecting antigen-antibody complexes formed. In one aspect, the method comprises contacting the biological sample with a peptide comprising an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 1-4, 275-412, and 421-423.

In other aspects, the present disclosure includes methods for diagnosing, prognosing or monitoring the treatment of a coronavirus infection in a subject by detecting in a sample from the subject the presence or absence of an antibody to a peptide comprising an amino acid sequence selected from the group consisting of SEQ ID NOS: 1-4 and SEQ ID NOS: 421-423. In various embodiments, methods for diagnosing, prognosing or monitoring the treatment of a coronavirus infection in a subject may comprise the step of detecting in a sample from the subject the presence or absence of an antibody to at least one of a first peptide, a second peptide, a third peptide, and a fourth peptide. The first peptide may comprise a first amino acid sequence comprising SEQ ID NO: 421; the second peptide may comprise a second amino acid sequence comprising SEQ ID NO: 422; the third peptide may comprise a third amino acid sequence comprising SEQ ID NO: 3; and the fourth peptide may comprise a fourth amino acid sequence comprising SEQ ID NO: 4. Further, methods of serologically detecting an antibody to coronavirus in a sample may comprise the steps of contacting the sample with a peptide under conditions sufficient to allow the binding of the antibody to the peptide, wherein the peptide comprises an amino acid sequence selected from the group consisting of SEQ ID NOS: 1-4 and SEQ ID NOS: 421-423, and detecting formation of an antibody-peptide complex comprising the antibody and the peptide. The formation of the antibody-peptide complex is indicative of an antibody to an epitope of a coronavirus antigen being present in the sample. In some embodiments, the coronavirus is severe acute respiratory syndrome coronavirus 2 (SARS-CoV-2).

The present disclosure further includes a peptide reactive with an antibody specific to coronavirus. The peptide may comprise an amino acid sequence selected from the group consisting of SEQ ID NOS: 1-4, 11, 13-15, 110-120, 275-412, and 421-423. In one aspect, the peptide comprises an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOS: 1-4, 11, 13-15, 110-120, 275-412, and 421-423. In some embodiments, a synthesized peptide is provided comprising an amino acid sequence selected from the group consisting of SEQ ID NOS: 1-4 and 421-423 or an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOS: 1-4 and 421-423. According to further embodiments, an immunogenic composition is provided. The immunogenic composition may comprise a peptide comprising an amino acid sequence selected from the group consisting of SEQ ID NOS: 1-4, 11, 13-15, 110-120, 275-412, and 421-423. The immunogenic composition may comprise a peptide comprising an amino acid sequence selected from the group consisting of SEQ ID NOS: 1-423. A method of eliciting an immune response in a subject may comprise the step of administering to the subject an immunogenic composition including a peptide comprising an amino acid sequence selected from the group consisting of SEQ ID NOS: 1-423, or from the group consisting of SEQ ID NOS: 1-4, 11, 13-15, 110-120, 275-412, and 421-423, or from the group consisting of SEQ ID NOS: 1-4 and 421-423. The immunogenic composition may further comprise a pharmaceutically acceptable carrier or adjuvant. Thus, a method of recruiting an antiviral antibody in a subject to prevent or limit a coronavirus infection in the subject is provided herein, wherein the antiviral antibody is specific to coronavirus.

In certain aspects, the present disclosure provides a method of detecting in a sample the presence of an antibody that binds to a spike protein or a nucleocapsid protein of a severe acute respiratory syndrome-associated coronavirus (SARS-CoV), the method comprising: providing a biological sample from a subject suspected to be infected with a SARS-CoV; contacting the biological sample with a peptide comprising an amino acid sequence selected from the group consisting of SEQ ID NOs: 1-4, 275-412, and 421-423; and detecting antigen-antibody complexes formed.

In one aspect, the method comprises the step of: detecting in the biological sample from the subject the presence or absence of an antibody to at least one of a first peptide, a second peptide, a third peptide, and a fourth peptide; wherein the first peptide comprises a first amino acid sequence comprising SEQ ID NO: 1; the second peptide comprises a second amino acid sequence comprising SEQ ID NO: 2; the third peptide comprises a third amino acid sequence comprising SEQ ID NO: 3; and the fourth peptide comprises a fourth amino acid sequence comprising SEQ ID NO: 4. In another aspect, the method comprises detecting in the biological sample from the subject the presence or absence of an antibody to the first peptide, second peptide, third peptide, and fourth peptide.

In some aspects, the method comprises the step of detecting in the biological sample from the subject the presence or absence of: a first antibody to a peptide comprising an amino sequence comprising SEQ ID NO: 421; a second antibody to a peptide comprising an amino sequence comprising SEQ ID NO: 422; and/or a third antibody to a peptide comprising an amino sequence comprising SEQ ID NO: 423. In one aspect, the method comprises detecting in the biological sample from the subject the presence or absence of the first antibody, the second antibody, and the third antibody.

In some aspects, the SARS-CoV is severe acute respiratory syndrome coronavirus 2 (SARS-CoV-2). In other aspects, the biological sample is whole blood, serum, or plasma. In yet other aspects, the method of detecting antigen-antibody complexes comprises a technique selected from the group consisting of flow cytometry, immunohistochemistry, enzyme-linked immunosorbent assay (ELISA), Western Blot, and immunoaffinity chromatography.

In certain aspects, the present disclosure relates to an immunogenic composition, comprising: a peptide comprising an amino acid sequence selected from the group consisting of SEQ ID NOs: 1-4, 275-412, and 421-423; and a pharmaceutically acceptable carrier or adjuvant. In other aspects, the present disclosure relates to an immunogenic composition, comprising: a peptide comprising an amino acid sequence selected from the group consisting of SEQ ID NOS: 424-447; and a pharmaceutically acceptable carrier or adjuvant. In yet other aspects, the present disclosure relates to an immunogenic composition, comprising: a peptide comprising an amino acid sequence selected from the group consisting of SEQ ID NOs: 1-4, 275-412, and 421-447 or an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 1-4, 275-412, and 421-447; and a pharmaceutically acceptable carrier or adjuvant.

In one aspect, the immunogenic composition comprises: a first peptide comprising an amino sequence comprising SEQ ID NO: 421; a second peptide comprising an amino sequence comprising SEQ ID NO: 422; and/or a third peptide comprising an amino sequence comprising SEQ ID NO: 423.

In another aspect, the immunogenic composition comprises: a first peptide comprising an amino sequence comprising SEQ ID NO: 435; a second peptide comprising an amino sequence comprising SEQ ID NO: 441; a third peptide comprising an amino sequence comprising SEQ ID NO: 442; and/or a fourth peptide comprising an amino sequence comprising SEQ ID NO: 447.

In one aspect, the immunogenic composition comprises: a first peptide comprising an amino sequence comprising SEQ ID NO: 1; a second peptide comprising an amino sequence comprising SEQ ID NO: 2; a third peptide comprising an amino sequence comprising SEQ ID NO: 3; and/or a fourth peptide comprising an amino sequence comprising SEQ ID NO: 4.

In another aspect, the immunogenic composition comprises: a first peptide comprising an amino sequence comprising SEQ ID NO: 422; a second peptide comprising an amino sequence comprising SEQ ID NO: 423; a third peptide comprising an amino sequence comprising SEQ ID NO: 435; a fourth peptide comprising an amino sequence comprising SEQ ID NO: 441; a fifth peptide comprising an amino sequence comprising SEQ ID NO: 442; and/or a sixth peptide comprising an amino sequence comprising SEQ ID NO: 447.

In other aspects, the present disclosure relates to a method of eliciting an immune response in a subject, the method comprising administering to the subject an immunogenic composition disclosed herein.

In certain aspects, the present disclosure provides a method of detecting in a sample the presence of an antibody that binds to a spike protein or a nucleocapsid protein of a severe acute respiratory syndrome-associated coronavirus (SARS-CoV), the method comprising: providing a biological sample from a subject suspected to be infected with a SARS-CoV; contacting the biological sample with a peptide comprising an amino acid sequence selected from the group consisting of SEQ ID NOs: 424-447; and detecting antigen-antibody complexes formed.

In one aspect, the method comprises the step of detecting in the biological sample from the subject the presence or absence of: a first antibody to a peptide comprising an amino sequence comprising SEQ ID NO: 435; a second antibody to a peptide comprising an amino sequence comprising SEQ ID NO: 441; a third antibody to a peptide comprising an amino sequence comprising SEQ ID NO: 442; and/or a fourth antibody to a peptide comprising an amino sequence comprising SEQ ID NO: 447.

In yet another aspect, the disclosure provides a method of detecting in a sample the presence of an antibody that binds to a spike protein or a nucleocapsid protein of a severe acute respiratory syndrome-associated coronavirus (SARS-CoV), the method comprising: providing a biological sample from a subject suspected to be infected with a SARS-CoV; contacting the biological sample with a peptide comprising an amino acid sequence selected from the group consisting of SEQ ID NOs: 1-4, 275-412, and 421-447 or an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 1-4, 275-412, and 421-447; and detecting antigen-antibody complexes formed.

In one aspect, the method comprises the step of detecting in the biological sample from the subject the presence or absence of: a first antibody to a peptide comprising an amino sequence comprising SEQ ID NO: 422; a second antibody to a peptide comprising an amino sequence comprising SEQ ID NO: 423; a third antibody to a peptide comprising an amino sequence comprising SEQ ID NO: 435; a fourth antibody to a peptide comprising an amino sequence comprising SEQ ID NO: 441; a fifth antibody to a peptide comprising an amino sequence comprising SEQ ID NO: 442; and/or a sixth antibody to a peptide comprising an amino sequence comprising SEQ ID NO: 447. In another aspect, the method comprises the step of detecting in the biological sample from the subject the presence or absence of the first antibody, second antibody, third antibody, fourth antibody, fifth antibody, and sixth antibody.

In some aspects, the present disclosure provides a method for diagnosing, prognosing or monitoring the treatment of a coronavirus infection in a subject, the method comprising the step of: detecting in a sample from the subject the presence or absence of an antibody to at least one of a first peptide, a second peptide, a third peptide, and a fourth peptide; wherein the first peptide comprises a first amino acid sequence selected from the group consisting of SEQ ID NOs: 1-4, 11, 13-15, 110-120, 275-412, and 421-423; wherein the second peptide comprises a second amino acid sequence selected from the group consisting of SEQ ID NOs: 1-4, 11, 13-15, 110-120, 275-412,and 421-423; wherein the third peptide comprises a third amino acid sequence selected from the group consisting of SEQ ID NOs: 1-4, 11, 13-15, 110-120, 275-412, and 421-423; wherein the fourth peptide comprises a fourth amino acid sequence selected from the group consisting of SEQ ID NOs: 1-4, 11, 13-15, 110-120, 275-412, and 421-423; and wherein the first peptide, second peptide, third peptide, and fourth peptide comprise different amino acid sequences.

In other aspects, the present disclosure provides a method of serologically detecting an antibody to coronavirus in a sample, the method comprising the steps of: contacting the sample with a peptide, under conditions sufficient to allow the binding of the antibody to the peptide, wherein the peptide comprises an amino acid sequence selected from the group consisting of SEQ ID NOs: 1-4, 11, 13-15, 110-120, 275-412, and 421-423; and detecting formation of an antibody-peptide complex comprising the antibody and the peptide, wherein formation of the antibody-peptide complex is indicative of an antibody to an epitope of a coronavirus antigen being present in the sample.

In some aspects, the amino acid sequence is selected from the group consisting of SEQ ID NOs: 1-4 and 421-423. In other aspects, the subject is human.

In other aspects, the present invention provides a peptide reactive with an antibody specific to coronavirus, where in the peptide comprises an amino acid sequence selected from the group consisting of SEQ ID NOs: 1-4, 11, 13-15, 110-120, 275-412, and 421-423. In one aspect, the present invention provides a peptide reactive with an antibody specific to coronavirus, where in the peptide comprises an amino acid sequence selected from the group consisting of 1-4, 275-412, and 421-447.

In other aspects, the present invention provides an immunogenic composition, comprising: a peptide comprising an amino acid sequence selected from the group consisting of SEQ ID NOs: 1-447.

In yet other aspects, the present disclosure relates to a method of eliciting an immune response in a subject, the method comprising: administering to the subject an immunogenic composition including a peptide comprising an amino acid sequence selected from the group consisting of SEQ ID NOs: 1-423. In one aspect, the present disclosure relates to a method of eliciting an immune response in a subject, the method comprising: administering to the subject an immunogenic composition including a peptide comprising an amino acid sequence selected from the group consisting of SEQ ID NOs: 1-447.

In some aspects, the present invention provides a method of recruiting an antiviral antibody in a subject to limit a coronavirus infection in the subject, the method comprising: administering a peptide to the subject, wherein the peptide comprises an amino acid sequence selected from the group consisting of SEQ ID NOs: 1-4, wherein the antiviral antibody is specific to coronavirus. In one aspect, the present invention provides a method of recruiting an antiviral antibody in a subject to limit a coronavirus infection in the subject, the method comprising: administering a peptide to the subject, wherein the peptide comprises an amino acid sequence selected from the group consisting of SEQ ID NOs: 1-4, 275-412, and 421-447; wherein the antiviral antibody is specific to coronavirus.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE FIGURES

Illustrative and exemplary embodiments of the invention are shown in the drawings in which:

FIGS. 1A-1D illustrate epitope-resolved CoV serology using a highly-multiplexed peptide-based assay.

FIGS. 2A-2C illustrate results of the peptide-based assay identifying recurrent reactivities to SARS-CoV-2 peptides and classifying exposure status with high accuracy.

FIGS. 3A-3C illustrate recurrent Spike protein epitopes correspond to accessible and functionally-important sites within the protein structure.

FIGS. 4A-4E illustrate recurrent SARS-CoV-2 epitopes correspond to conserved regions of Spike S2 that are also targeted in the response to other CoVs.

FIGS. 5A-5D illustrate Spike HR2 antibodies elicited by SARS-CoV-2 strongly cross-react with the homologous region of Betacoronavirus 1.

FIG. 6 illustrates the strong correlation between replicate PepSeq assays run separately on the same serum sample.

FIG. 7 illustrates the distribution of enriched peptides from CoV Spike proteins in the HV library.

FIGS. 8A-8C illustrate the effect of age and sample source on number of enriched control peptides.

FIGS. 9A-9E illustrate the identification of recurrent reactivities to SARS-CoV-2 peptides and classification of exposure status with PepSeq.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

Generally, some embodiments of the present invention can be used to develop diagnostics, therapies, and vaccines for coronavirus infection based on the peptides and epitopes identified in the present disclosure. Among the various aspects of the present invention is the provision of one or more targets for diagnosing, treating, and preventing coronavirus infection in a subject. The disclosed epitope regions of coronaviruses, including SARS-CoV-2, indicate uses including serological assays, vaccines for eliciting antibodies, and predicting an individual's antibody response to a coronavirus infection.

The invention may comprise methods for detecting the presence of, exposure to, or infection by a particular virus in a sample. The invention may further comprise methods of population screening, predicting immune response or disease outcome in an individual, serological assays, and neutralization assays. The invention may further comprise a method of eliciting neutralization antibodies to coronavirus.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print, or any other material isolated in whole or in part from a living subject or organism. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

The terms “peptide,” “polypeptide,” and “protein” are used interchangeably herein to refer to polymers of amino acids of any length, chemically or biochemically modified or derivatized amino acids, and polypeptides having modified peptide backbones. These terms also include proteins that are post-translationally modified through reactions that include glycosylation, acetylation and phosphorylation. The term “at least a portion” of a polypeptide means a portion having the minimal size characteristics of such sequences, or any larger fragment of the full length molecule, up to and including the full length molecule. For example, a portion of a polypeptide may be 4 to 15 amino acids, or may be 4 amino acids, 5 amino acids, 6 amino acids, 7 amino acids, and so on, up to a full length polypeptide. A portion of a polypeptide useful as an epitope may be as short as 4 amino acids. A portion of a polypeptide that performs the function of the full-length polypeptide would generally be longer than 4 amino acids.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified. Unnatural amino acids are not encoded by the genetic code and can, but do not necessarily have the same basic structure as a naturally occurring amino acid. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to by either the three letter symbols or by the one-letter symbols recommended by the IUPAC, the IUAPC letter code are as follows: G=Glycine; A=Alanine; L=Leucine; M=Methionine; F=Phenylalanine; W=Tryptophan; K=Lysine; Q=Glutamine; E=Glutamic Acid; S=Serine; P=Proline; V=Valine; I=Isoleucine; C=Cysteine; Y=Tyrosine; H=Histidine; R=Arginine; N=Asparagine; D=Aspartic Acid; T=Threonine.

The terms “homologous” and “similar” refer to the relationship between proteins that possess a “common evolutionary origin,” including proteins from superfamilies (e.g., the immunoglobulin superfamily) and homologous proteins from different species. Such proteins (and their encoding genes) have sequence homology, as reflected by their sequence similarity, whether in terms of percent similarity or the presence of specific residues or motifs as conserved positions. In a specific embodiment, two peptide sequences are “substantially homologous or similar” when at least about 80%, or at least about 90%, or at least about 95) of the amino acids match over the defined lengths of the amino acid sequences.

The term “variant” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Variants may include individual substitutions, deletions or additions to a nucleic acid, peptide, polypeptide, or protein sequence which alters, adds or deletes a single amino acid or a small percentage of amino acids in the encoded sequence.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein so that the percent protein or amino acid sequence similarity between any two proteins of similar function may vary and may be, for example, from 70% to 99% as determined according to an alignment scheme. A “variant” also includes a polypeptide which has at least 60% amino acid identity as determined by BLAST or FASTA algorithms, preferably at least 75% most preferably at least 85%, and even more preferably at least 90%, and still more preferably at least 95%, and which has the same or substantially similar properties or functions as the native or parent protein to which it is compared. A particular variant is a “gain-of-function” variant, meaning a polypeptide variant in which the change of at least one given amino acid residue in a protein or enzyme improves a specific function of the polypeptide, including, but not limited to protein activity. The change in amino acid residue can be replacement of an amino acid with one having similar properties.

The term “antibody” is used herein in the broadest sense and refers generally to a molecule that contains at least one antigen binding site that immunospecifically binds to a particular antigen target of interest. The term “antibody” thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term “antibody,” thus, includes full length antibodies and/or their variants, as well as fragments thereof. Binding of an antibody to a target can cause a variety of effects, such as but not limited to where such binding modulates, decreases, increases, antagonizes, agonizes, mitigates, alleviates, blocks, inhibits, abrogates and/or interferes with at least one target activity or binding, or with receptor activity or binding, in vitro, in situ, and/or in vivo.

Antibodies (Abs) can be assigned to different classes, which differ in their biological properties, functional locations, and ability to deal with different antigens. There are five major classes of immunoglobulins (Ig): IgA, IgD, IgE, IgG, and IgM, and several of these may be further divided into subclasses (isotypes), e.g., IgG1, IgG2, IgG3, IgG4, IgA, and IgA2. The classes of immunoglobulins differ in their biological properties, functional locations, and interactions with different antigens. Immunoglobulin G (IgG), the major antibody in serum, is a heterotetrameric protein with two heavy chains and two light chains. IgG can be cleaved into three 50-kd fragments: two Fab fragments that bind antigen (F=fragment, ab=antigen binding), and an Fc fragment that crystallizes readily and does not bind antigen. The Fab fragment also contains the constant domain of the light chain and the first constant domain (CH1) of the heavy chain. Fab fragments differ from Fab′ fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “antigen” refers to any substance that triggers the immune system to produce antibodies against it. An antigen acts by stimulating the proliferation of the small number of cells that were already forming an antibody capable of recognizing the antigen.

Antibodies have specific and high affinity for the antigens that elicited their synthesis. Synthetic peptides can serve as antigens to stimulate the formation of specific antibodies. An antibody recognizes, on a large molecule, a specific group or cluster of amino acids called an epitope.

As used herein, the term “epitope” refers to a portion of an antigenic molecule to which an antibody is produced and to which the antibody will bind. For example, a “Coronavirus epitope” comprises the part of a coronavirus protein to which an anti-coronavirus antibody specifically binds. Epitopes can comprise linear amino acid residues (i.e., residues within the epitope are arranged sequentially one after another in a linear fashion), nonlinear amino acid residues, or both linear and nonlinear amino acid residues.

The terms “immunogen” and “immunogenic” refer to any compound or substance that is capable of eliciting an immune response in a human or non-human individual to the compound or substance, such as an antigen.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex.

As used herein, the term “specific binding” refers to the specificity of a binder, e.g., a protein or an antibody, such that it preferentially binds to a target, such as a polypeptide antigen, a receptor, or an antibody. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments, peptides, or fusion peptides that are specific for or bind specifically to a target bind to the target with higher affinity than binding to other non-target substances. Also preferably, binders, antibodies or antibody fragments, peptides, or fusion peptides that are specific for or bind specifically to a target avoid binding to a significant percentage of non-target substances, e.g., non-target substances present in a testing sample. The binding affinity of an antibody to a target antigen, antigenic fragment, peptide, or fusion peptide, comprising the cognate epitope can be readily determined using any of a number of methods available in the art including, but not limited to, enzyme linked immunosorbent assay (ELISA). In some embodiments, binders, antibodies or antibody fragments, peptides, or fusion peptides of the present disclosure avoid binding greater than about 90% of non-target substances, although higher percentages are clearly contemplated and preferred. For example, binders, antibodies or antibody fragments, peptides, or fusion peptides of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments, peptides, or fusion peptides of the present disclosure avoid binding greater than about 10%, 20%, 30%, 40%, 50%, 60%, or 70%, or greater than about 75%, or greater than about 80%, or greater than about 85% of non-target substances.

A target or a marker may be any molecular structure produced by a cell, expressed inside the cell, accessible on the cell surface, or secreted by the cell. A marker may be any protein, carbohydrate, fat, nucleic acid, catalytic site, or any target of these such as an enzyme, glycoprotein, cell membrane, virus, cell, organ, organelle, or any uni-or multimolecular structure or any other such structure now known or yet to be disclosed whether alone or in combination. A target may also be called a marker and the terms are used interchangeably.

A target may be represented by the sequence of amino acids, or sequence of one or more strands of a nucleic acid from which it may be derived. For example, a target may be represented by a protein sequence. Alternatively, a target may be represented by a nucleic acid sequence, the protein or peptide or the fragments thereof encoded by the nucleic acid sequence. Examples of such nucleic acids include both single stranded and double stranded nucleic acid sequences including miRNA, tRNA, siRNA, mRNA, cDNA, or genomic DNA sequences including complimentary sequences. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The term “target” further encompasses the products (i.e., proteins) of the gene or a gene allele thereof, whose expression or activity is directly or indirectly associated with a particular phenotype or cellular condition, or physiological characteristic.

Indirect methods of detecting a marker generally involve assessing the expression of material created from a genomic DNA template such as an RNA or protein molecule. Such expression may be assessed by any of a number of methods used currently in the art and yet to be developed. Examples include any nucleic acid detection method including the following nonlimiting examples, microarray RNA analysis, RNA in situ hybridization, RNAse protection assay, Northern blot, reverse transcription PCR, and quantitative reverse transcription PCR. Other examples include any process of detecting expression that uses an antibody including the following nonlimiting examples, flow cytometry, immunohistochemistry, ELISA, Western blot, Northwestern blot, and immunoaffinity chromatography. Antibodies may be monoclonal, polyclonal, or any antibody fragment including a Fab, F(ab)₂, Fv, scFv, phage display antibody, peptibody, multispecific ligand, or any other reagent with specific binding to a target. Other methods of assessing protein expression include the following nonlimiting examples: HPLC, mass spectrometry, protein microarray analysis, PAGE analysis, isoelectric focusing, 2-D gel electrophoresis, and enzymatic assays.

One aspect of the disclosure encompasses methods of eliciting an immune response in a subject by administering to the subject an immunogenic composition. The methods may include recruiting an antiviral antibody in the subject, to protect the subject against a coronavirus infection. The methods, vaccines, and therapeutics may include including a peptide comprising an amino acid sequence selected from the group consisting of SEQ ID NOS: 1-4 and 421-423. The methods, vaccines, and therapeutics may include, for example, a monoclonal antibody produced using the peptides disclosed herein.

Agents that interact with a therapeutic target to result in a desirable therapeutic effect may include a pharmaceutically active ingredient or pharmaceutically acceptable salt thereof, a drug, a toxin, a chemical, a small organic molecule, a large molecule or peptide or an antibody. Large-molecule pharmaceuticals refer to pharmaceutical agents having a molecular weight greater than about 1000 Daltons, e.g., peptidic drugs, vaccines and hormones. The term “antibody” is used herein in the broadest sense and refers generally to a molecule that contains at least one antigen binding site that immunospecifically binds to a particular antigen target of interest. Antibody thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term, thus, includes full length antibodies and/or their variants as well as immunologically active fragments thereof, thus encompassing, antibody fragments capable of binding to a biological molecule (such as an antigen or receptor) or portions thereof, including, but not being limited to, Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

The screening or creation, identification and selection of appropriate therapeutic agent through the target identified herein can be accomplished by a variety of methods. One approach is to use structural knowledge about the target protein to design a candidate molecule with which it will precisely interact. An example would be computer assisted molecular design. A second approach is to use combinatorial or other libraries of molecules, whereby a large library of molecules is screened for inhibitory effect with regard to the target gene or protein expression, or ability to inhibit the transcriptional factor activity of the target protein. In a further example, a panel of antibodies may be screened for their ability to inhibit the target protein.

The term “monoclonal antibody” as used herein refers to an antibody obtained from a population of substantially homogeneous antibodies, i.e., the individual antibodies comprising the population are identical except for possible naturally occurring mutations that may be present in minor amounts. Various procedures known in the art may be used for the production of polyclonal or monoclonal antibodies to Coronavirus polypeptides or derivatives or analogs thereof. For the production of antibodies, various host animals can be immunized by injection with the antigenic polypeptide, including but not limited to rabbits, mice, rats, sheep, goats, etc. For preparation of monoclonal antibodies directed toward the Coronavirus polypeptides, any technique that provides for the production of antibody molecules by continuous cell lines in culture may be used. These include but are not limited to the hybridoma technique originally developed by Kohler and Milstein (Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al., in Monoclonal Antibodies and Cancer Therapy, Alan R. Liss, Inc., pp. 77-96, 1985).

In the preparation of the pharmaceutical compositions comprising the antibodies described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

In other embodiments there is provided a pharmaceutical composition including an antibody or fragment as described above together with a pharmaceutically acceptable carrier, diluent or excipient. As used herein, “carrier(s)” can be used interchangeably with “excipient(s)” Carriers include any substance that may be administered with the one or more disclosed compounds with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils). Thus, the pharmaceutical compositions may generally comprise a pharmaceutically acceptable carrier and a pharmacologically effective amount of the antibodies, or mixture of antibodies. The pharmaceutical composition may be formulated as powders, granules, solutions, suspensions, aerosols, solids, pills, tablets, capsules, gels, topical creams, suppositories, transdermal patches, and other formulations known in the art.

The pharmaceutical compositions described herein may be administered by any means that enables the active agent to reach the agent's site of action in the body of the subject. The dosage administered varies depending upon factors, such as: pharmacodynamic characteristics; mode and route of administration; age, health, and weight of the recipient subject; nature and extent of symptoms; concurrent treatments; and frequency of treatment.

As used herein, the terms “administration” and “administering” of an agent to a subject include any route of introducing or delivering the agent to a subject to perform its intended function. Administration can be carried out by any suitable route, including intravenously, intramuscularly, intraperitoneally, inhalationally, intranasally, or subcutaneously. Administration includes self-administration and the administration by another.

The term “effective amount” or “therapeutically effective amount” refers to that amount of an agent or combination of agents as described herein that is sufficient to effect the intended application including, but not limited to, disease treatment and/or disease prevention. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “patient” or “subject” refers to any organism to which a provided composition is or may be administered, e.g., for experimental, diagnostic, prophylactic, cosmetic, and/or therapeutic purposes. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human.

The following examples are given for illustrative and non-limiting purposes of the present invention.

### EXAMPLES

In this example, a multiplexed peptide assay (‘PepSeq’) was used to generate an epitope-resolved view of antibody reactivity across all human coronaviruses (CoVs). PepSeq accurately classified SARS-CoV-2 exposure status and revealed epitopes across the Spike and Nucleocapsid proteins. Two of these epitopes represent recurrent reactivities to conserved, functionally-important sites in the S2 subunit of Spike, regions that we show are also targeted for the endemic CoVs in pre-pandemic controls. At one of these sites, we demonstrate that the SARS-CoV-2 response strongly and recurrently cross-reacts with the endemic virus hCoV-OC43. Our epitope-resolved analysis reveals new targets for the development of diagnostics, vaccines and therapeutics, including a site that may have broad neutralizing potential.

### Methods

### Samples

COVID-19 convalescent serum and plasma samples were collected at four clinical sites from patients who had tested positive for SARS-CoV-2 by RT-PCR a median of 28.5 days prior. Pre-pandemic negative control samples were collected from two sites. Pre-pandemic negative control serum samples were characterized using our SCV2 library (n=17). These samples were collected during January 2015 from multiple locations in California. Pre-pandemic negative control serum samples characterized using our HV library (n=33) were collected during 2019 (latest collections were during the first week of December). Serum was obtained from whole blood using lithium heparin gel plasma separator tubes followed by centrifugation.

### PepSeq Library Design

We designed two different libraries of peptides in order to assess antibody reactivity to SARS-CoV-2 peptides and to peptides from other human-infecting coronaviruses. The first set of peptides, referred to herein as the “human virome” (HV) peptides, was designed to broadly cover potential epitope diversity for all viruses known to infect humans. To generate this design, we downloaded all protein sequences available in UniProt, on Nov. 19, 2018, that were linked to 474 viral species-level taxonomy IDs. Following a series of quality filters to remove identical sequences, those that were too short (<30 aa), those that contained recombinant non-viral sequences and those that were taxonomically misclassified, we were left with 1,300,994 target protein sequences. In order to control for sampling bias within the database, we randomly subsampled overrepresented virus species, including no more than 2000 and 4000 sequences for viruses with RNA and DNA genomes, respectively. Additional protein sequences were allowed for DNA viruses because they often contain larger genomes and proteomes (i.e., more distinct genes). When down-sampling, priority was given to proteins from the Swiss-Prot database, which have been manually reviewed. The final down-sampled target set included 148,215 protein sequences and 88.78 M amino acids.

Our HV peptides were designed using an epitope-centric set cover design algorithm, with a focus on optimizing 9mer (i.e., 9 amino acid long) epitope coverage using 30mer peptides. To reduce the runtime and memory requirements of the algorithm, we partitioned our target protein sequences according to taxonomy. Given the high levels of genetic divergence between viral families and genera, we do not expect that this partitioning substantially impacted our final design. Including a small set of negative control peptides selected from eukaryotic proteins, this design included 244,000 unique 30mer peptides, and represents approximately 70% of all potential 9mer epitopes contained within the target protein sequences. Each of these peptides was represented by a single nucleotide encoding. This design does not contain any peptides derived from SARS-CoV-2, but does contain full proteome coverage of the other six coronaviruses known to infect humans: Human coronavirus 229E (NCBI taxID: 11137), Human coronavirus NL63 (NCBI taxID: 277944), Human coronavirus HKU1 (NCBI taxID: 290028), Betacoronavirus 1 (NCBI taxID: 694003, includes Human coronavirus OC43), Severe acute respiratory syndrome-related coronavirus (NCBI taxID: 694009, “SARS”), and Middle East respiratory syndrome-related coronavirus (NCBI taxID: 1335626, “MERS”).

Our second design (SCV2) focused almost entirely on SARS-CoV-2, including high density tiling of peptides across the two most immunogenic SARS-CoV-2 proteins: the spike glycoprotein(S) and the nucleocapsid protein (N). As targets for this design, we utilized 2303 SARS-CoV-2 genome sequences downloaded from GISAID on Apr. 3, 2020, along with six locally generated sequences. Using these genomes, we first generated consensus amino acid sequences for the S and N proteins. In our design, we included all of the unique 30mer peptides contained in these consensus sequences, equivalent to a 1-step sliding window approach (Shiryaev et al., 2012). Additionally, we used the same epitope-centric set cover design algorithm used for HV in order to capture amino acid-level polymorphisms present within our full set of target genomes. This aspect of the design ensured that 100% of the unique 16mer peptides present in the S and N proteins from the 2309 SARS-CoV-2 genomes were represented in our design. In total, this design included 1550 30mer peptides from the S protein and 557 30mer peptides from the N protein. Each of these peptides was represented by three different nucleotide encodings. This design also included a set of 373 control peptides. These controls represent a subset of the HV peptides, which we have determined are commonly recognized by IgG antibodies in human sera (unpublished results). Therefore, we expect that some fraction of these controls will be recognized by antibodies in each blood sample tested. Collectively, these peptides were designed from 55 different virus species, including the four endemic human coronaviruses.

### PepSeq Library Synthesis and Assay

Libraries of covalently-coupled peptide: DNA conjugates were prepared from pools of DNA oligonucleotide templates in bulk enzymatic reactions using the method described previously (Kozlov et al., 2012), with minor modifications as noted hereafter. Briefly, pools of ssDNA templates (Agilent) were PCR-amplified and the dsDNA products were used as templates for in vitro transcription (Ampliscribe). The resulting mRNA was ligated to a hairpin oligonucleotide adapter bearing a puromycin molecule tethered by a PEG spacer and, following buffer exchange, the reaction mix was used as a template in an in vitro translation reaction (PURExpress, NEB). Constructs bearing mRNA—comprising of (i) mRNA, (ii) mRNA+adapter, (iii) mRNA+adapter+peptide—were isolated using magnetic beads coated with a DNA oligo complementary to a 30-mer sequence in the mRNA constant region. A reverse transcription reaction, primed by the adapter hairpin, was used to generate cDNA, after which RNase was applied to remove mRNA. Product was buffer-exchanged, quantified by running on a gel against standard DNA oligos of known concentrations, and used without further modifications or purification.

To perform serological assays, 5 uL of a 1:10 dilution of serum in Superblock T20 (Thermo) was added to 0.1 pmol of PepSeq library for a total volume of 10 uL and was incubated at 20° C. overnight. The binding reaction was applied to pre-washed protein G-bearing beads (Thermo) for 15 minutes, after which beads were washed 10 times with 1×PBST (washing steps were performed on an EpMotion robot, Eppendorf). After the final wash, beads were resuspended in 30 uL of water and heated to 95° C. for 5 minutes to elute bound product. Elutions were amplified and indexed using barcoded DNA oligos. Following PCR cleanup, products were pooled, quantified and sequenced on a NextSeq instrument (Illumina).

### PepSeg Data Analysis

We used PepSIRF v1.3.0 (Fink et al., 2020), along with custom scripts, to analyze the PepSeq HTS data. The data analysis included three primary steps: 1) demultiplexing and assignment of reads to peptides, 2) calculation of enrichment Z-scores individually for each assay and peptide and 3) identification of enriched peptides for each sample based on the consistency of Z-scores across replicates.

Demultiplexing and assignment of reads to peptides was done using the demux module of PepSIRF (Fink et al., 2020), allowing up to 1 mismatch within each of the index sequences and up to 2 mismatches with the expected DNA tag (90 nt in length). Z-scores were calculated using a method adapted from (Mina et al., 2019). This process involved the generation of peptide bins, each of which contained ≥300 peptides with similar starting abundance in our PepSeq assay. Starting abundance for each peptide was estimated using buffer-only controls. In total, 4-8 independent buffer-only controls were used to generate the bins for this study. The raw read counts from each of these controls were first normalized to reads per million (RPM) using the column sum normalization method in the norm module of PepSIRF. This was to ensure that independent assays were weighted evenly, regardless of differences in the depth of sequencing. Bins were then generated using the bin PepSIRF module.

Z-scores were calculated using the zscore module of PepSIRF, and each Z-score corresponds to the number of standard deviations away from the mean, with the mean and standard deviation calculated independently for the peptides from each bin. It is important that the mean and standard deviation reflect the distribution of unenriched peptides within a bin. Therefore, these calculations were based on the 75% highest density interval of read counts within each bin. Prior to Z-score calculation, RPM counts for each peptide were further normalized by subtracting the average RPM count observed within our superblock-only controls. This second normalization step controlled for variability in peptide starting abundance within a bin. Finally, the “p_enrich” module of PepSIRF was used to determine which peptides had been enriched through our assay. This module identifies peptides that meet or exceed minimum thresholds, in both replicates, for Z-score and normalized read count. Decision tree analysis was conducted using the Decision TreeClassifier method in the Scikit-learn Python module, v0.20.1.

### Visualization of Protein Structure

To visualize our identified SARS-CoV-2 epitopes within the 3D conformational structure of the S protein, we utilized the cryo-electron microscopy (Cryo-EM) structure available in the RCSB Protein Data Bank (PDB id: 6VY). To compare epitope positions across CoV species, we built three additional structures using Cryo-EM templates from PDB: 5SZS for hCoV-NL63, 6ACD for SARS-CoV and 6NZK for hCoV-OC43. We performed structural modelling using Swiss-Model software (Waterhouse et al., 2018). Structural alignments and image preparation were done with PyMOL (version 2.3.2, Schrodinger, LLC). For positioning of epitopes in the structures with respect to the SARS-CoV-2 spike protein, we performed corresponding amino acid sequence alignments with Clustal Omega. To build models of the post-fusion state for S2 subunit fragments, we used the Cryo-EM structure for murine SARS-CoV, determined by Walls et al. (PDB id: 6B30) (Walls et al., 2017).

### Results

### A Highly-Multiplexed Peptide Assay to Evaluate CoV Antibody Responses

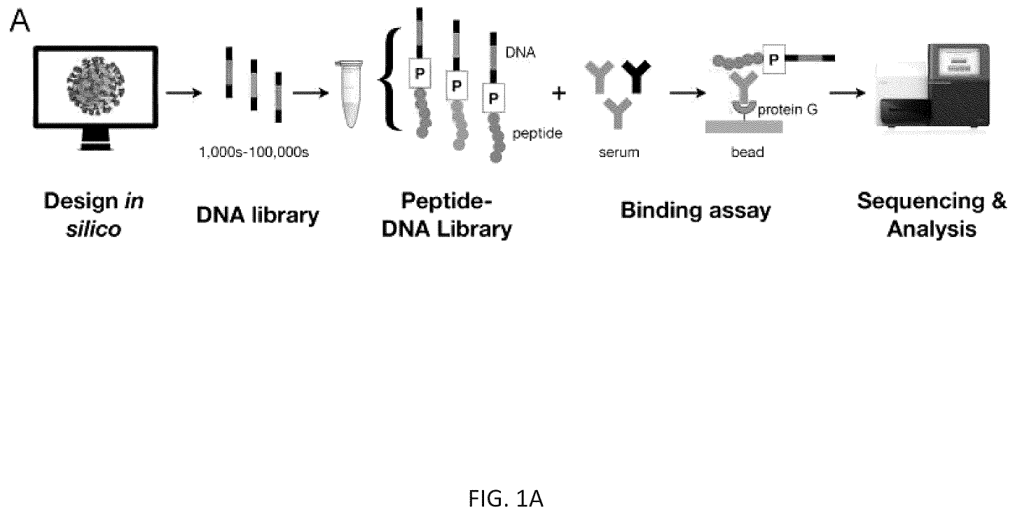

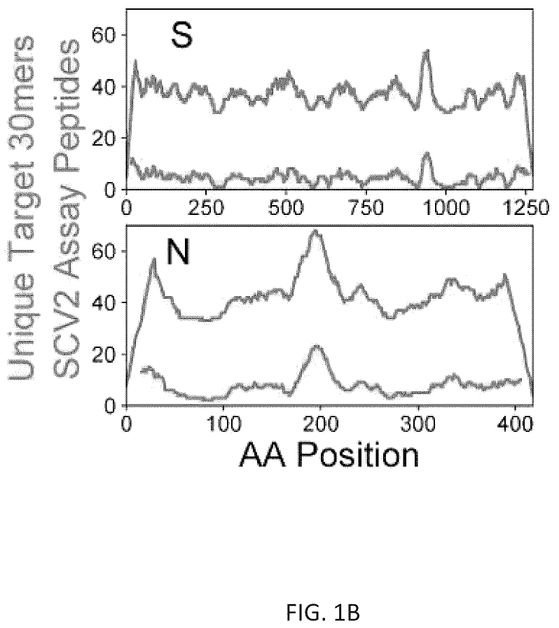

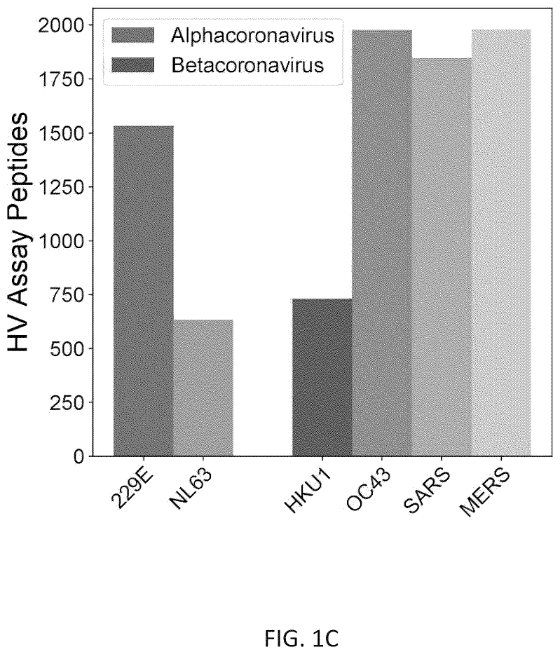

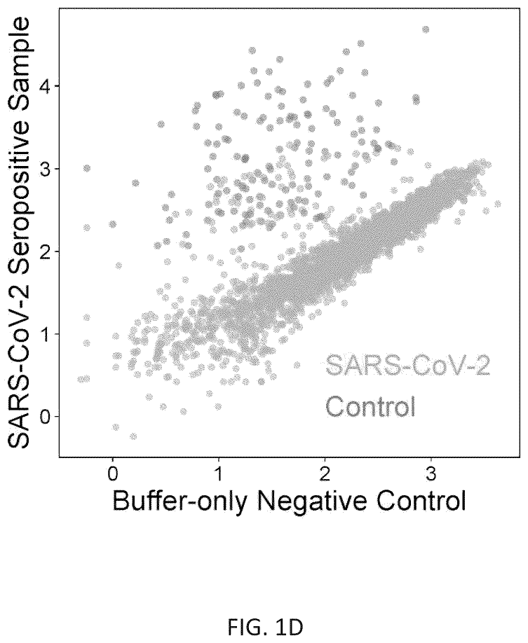

To generate a broad and high-resolution view of the antibody response to human coronaviruses, including SARS-CoV-2, we designed and synthesized two separate DNA-barcoded 30mer peptide libraries, using the method described previously (Kozlov et al., 2012) (FIG. 1A). FIG. 1A shows a method and platform for customizable highly-multiplexed peptide-based serology, comprising the following steps: (i) in silico design, (ii-iii) generation of a library of DNA-barcoded peptides from oligonucleotide templates using bulk in vitro reactions (transcription, ligation of a Puromycin (P)-containing adapter, translation, reverse transcription), (iv) serum binding assay and protein G capture, and (v) sequencing and analysis of the distribution of binders by their DNA barcodes.

Each library began as a pool of DNA oligonucleotide templates, which was modified using bulk enzymatic steps consisting of transcription, ligation of a puromycin-containing adapter oligo, translation, and reverse transcription. One library was focused on SARS-CoV-2 (‘SCV2’) and contained 2,107 peptides representing the Spike and Nucleocapsid—the 2 most immunogenic coronavirus proteins—at high redundancy, with an average of 38 peptides covering each amino acid position (FIG. 1B). FIG. 1B shows peptide coverage depth across the SARS-CoV-2 spike(S) and nucleocapsid (N) proteins within the ‘SCV2’ peptide library. Peptide coverage depth (blue) correlates well with amino acid sequence diversity within the target SARS-CoV-2sequences (green), calculated as the number of unique 30mers.

The other library (human virome or ‘HV’) comprised 244,000 peptides designed to cover the full proteomes of all viruses known to infect humans, as of the end of 2018. Therefore, HV included peptides from the complete proteomes of 6/7 human coronaviruses: HCoV-229E, HCoV-OC43, HCoV-NL63, HCoV-HKU1, SARS-CoV, and MERS-CoV, but not SARS-CoV-2 (FIG. 1C). FIG. 1C shows the number of peptides within the HV pool that were designed from each of the six human coronaviruses known prior to 2019. The SCV2 library also included 393 positive control peptides that we have previously shown are commonly recognized across the human population (unpublished data). The positive controls represent a subset of the HV peptides and were designed from 55 different virus species. In total, we assayed and analyzed 27 COVID-19 convalescent and 21 SARS-CoV-2 negative (both pre-and post-pandemic) serum samples using our SCV2 PepSeq library (TABLE 1).

TABLE 1
Summary of samples characterized in this study.

| | | | | | Median |
|---|---|---|---|---|---|
| PepSeq | Sample | Sample | Males/Females/ | Median | days from |
| Library | Type | Size | Unreported | Age* | diagnosis* |
| SCV2 | COVID-19 | 27 | 10/12/5 | 51 | 28.5 |
| | Convalescent | | | | |
| | Negative | 21 | 11/6/4 | 37 | — |
| | Control | | | | |
| HV | Negative | 33 | 0/0/33 | NA | |
| | Control | | | | |

“NA” = Not available;
“—” = Not applicable.
*Median values were calculated from a subset of total samples for which this information was available.

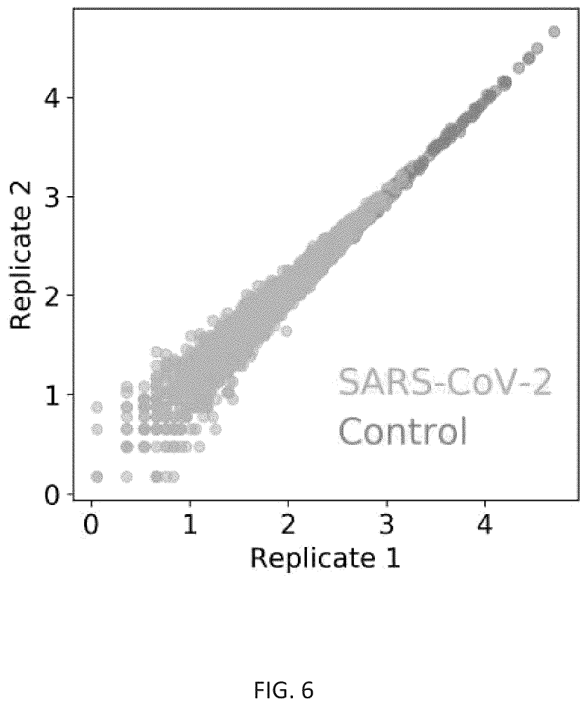

Separately, we assayed 33 SARS-CoV-2 negative (pre-pandemic) serum samples using the HV PepSeq library. For each assay, we incubated our PepSeq probes overnight with serum (or buffer as a negative control), captured the IgG on protein G beads, washed away the non-binding library members, eluted binders, and then performed PCR and high-throughput sequencing on the DNA tags to identify the distribution of bound peptides. Each sample was run in duplicate, and we observed strong signal concordance between technical replicates of the same sera, including those run on different days (FIG. 6). FIG. 6 shows a strong correlation between replicate PepSeq assays run separately on the same serum sample. Axes show normalized read counts (log10 scale) for each peptide in the SCV2 library. Grey circles represent unenriched peptides. Colored circles represent SARS-CoV-2 (orange) and non-SARS-CoV-2 control (blue) peptides that have been enriched through interaction with serum antibodies.

Comparative analysis of peptide abundance between serum and buffer-only negative controls revealed a strong correlation in abundance for the majority of peptides, while a subset of peptides showed distinctly higher relative abundance in each serum sample (FIG. 1D). These latter peptides are those that have been enriched by binding to serum IgG. To quantify peptide enrichment, we calculated Z-scores for each peptide in each sample. For each peptide, relative abundance was normalized to the corresponding value for the buffer-only negative controls, and this normalized value was compared among peptides with similar abundance in the negative controls. Each Z-score corresponds to the number of standard deviations away from the mean.

FIG. 1D shows an example scatter plot illustrating SCV2 PepSeq assay results for a single serum sample. This plot shows normalized sequence read counts (log10 scale) for each peptide in the SCV2 library. Assay results using an antibody-free negative control are shown on the x-axis, while the results from a SARS-CoV-2 convalescent serum sample are shown on the y-axis. Grey circles represent unenriched peptides, with a strong correlation between the two assays, based on the starting abundance of the different peptides. Colored circles represent SARS-CoV-2 (orange) and non-SARS-CoV-2 control (blue) peptides that have been enriched through interaction with serum antibodies.

### Accurate Detection of SARS-CoV-2 Exposure and Identification of Epitopes

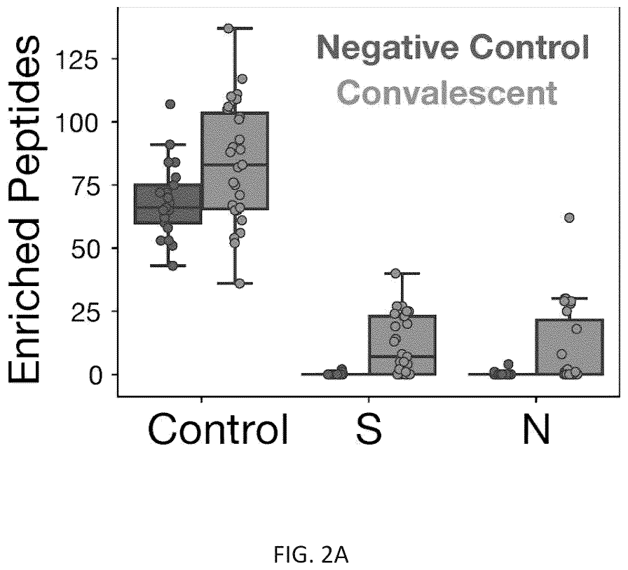

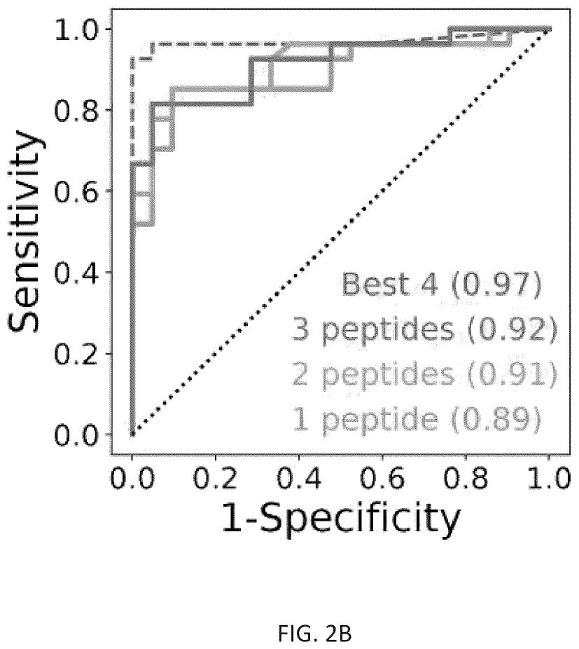

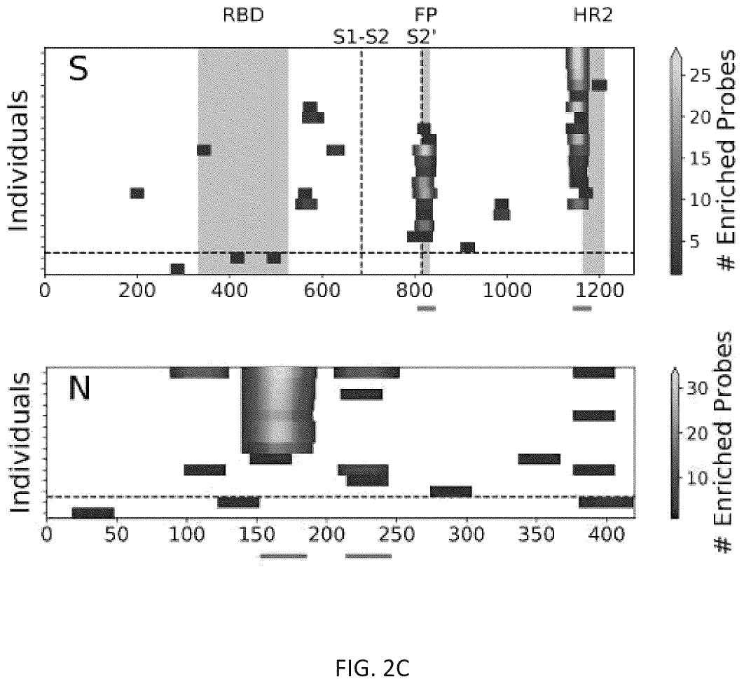

For the SCV2 PepSeq library, we evaluated the sensitivity/specificity for detection of SARS-CoV-2 exposure by generating receiver operating characteristic (ROC) curves with a sliding Z-score threshold and three different criteria for the number of enriched SARS-CoV-2 peptides needed for a positive result (FIG. 2B). The SCV2 assay distinguished COVID-19 convalescent samples from negative controls with high accuracy (AUC=0.89-0.92).

FIG. 2B shows ROC curves for prediction of SARS-CoV-2 exposure based on peptide-level Z-scores calculated for all SCV2 library peptides (solid lines) and for a subset of four peptides identified through a decision tree analysis (dashed line). Positivity of the assay was determined by the enrichment of peptides designed from SARS-CoV-2, and the full library analysis was run with three different thresholds for the number of enriched peptides required for a sample to be considered positive. For the analysis using only the “Best 4” peptides, only a single enriched peptide was required for a positive result. For all analyses, the AUC (shown in parentheses in FIG. 2B) was ≥0.89.

Based on the ROC analysis and a qualitative assessment of the ability to discriminate signal from noise (FIG. 1C), we selected a Z-score threshold of 11 for identifying enriched peptides; a peptide was required to meet or exceed this threshold in two technical replicates to be considered enriched. With only one SARS-CoV-2 peptide required for positivity, this threshold corresponded to a sensitivity of 81.5% and a specificity of 91.5%, with five false negative samples and two false positive samples. Notably, while both false positive samples exhibited at least one enriched peptide for both the N and S proteins, none of these peptides corresponded to the widely recognized, immunodominant epitopes observed for the COVID-19 convalescent sera (FIG. 2C). FIG. 2C shows heat maps showing the locations of enriched SARS-CoV-2 peptides within the S and N proteins. Each row represents a single serum sample and each plot includes only samples with at least one enriched peptide. Each position is colored according to the number of enriched peptides that overlap that position. The horizontal dashed line separates SARS-CoV-2 convalescent samples (top) from negative control samples (bottom). The vertical dashed lines in the S protein plot represent the S1-S2 and S2′ cleavage sites, respectively. Grey boxes indicate selected functional regions: receptor binding domain (RBD), fusion peptide (FP) and heptad repeat 2 (HR2). The horizontal green lines below each plot in FIG. 2C indicate the positions of the “Best 4” peptides from FIG. 2B.

To explore the potential for increasing sensitivity and specificity using a subset of SARS-CoV-2 peptides, we utilized a decision tree algorithm to identify the most discriminatory subset of peptides from our library. This analysis identified four SARS-CoV-2 peptides (indicated by green lines in FIG. 2C) that were sufficient to detect all 22 convalescent donors that were called positive using the entire peptide set (TABLE 2).

TABLE 2
SARS-CoV-2 peptides chosen by decision tree
algorithm for discriminating between COVID-19
convalescent and negative control samples

| | | | | Reactive | SEQ |
|---|---|---|---|---|---|
| | | Start | End | conv | ID |
| Peptide Sequence | Protein | position | position | samples | NO: |
| SFKEELDKYFKNHTSPDVDLGDISGINAS | S | 1147 | 1176 | 12 | 1 |
| V | | | | | |
| SKPSKRSFIEDLLFNKVTLADAGFIKQYG | S | 810 | 839 | 9 | 2 |
| D | | | | | |
| NAAIVLQLPQGTTLPKGFYAEGSRGGSQ | N | 154 | 183 | 8 | 3 |
| AS | | | | | |
| GDAALALLLLDRLNQLESKMSGKGQQQ | N | 215 | 744 | 3 | 4 |
| QGQ | | | | | |

Using only these four peptides in the ROC analysis of all 48 donors increased the AUC to 0.97. With the same Z-score threshold of 11, the specificity increased to 100%, while sensitivity stayed at 81.5% (FIG. 2B).

As predicted, multiple positive control peptides were found to be enriched in every serum sample that we tested (FIG. 2A). FIG. 2A shows boxplots showing the number of enriched SCV2 library peptides from assays with negative control (blue, n=21) and SARS-CoV-2 convalescent (orange, n=27) serum samples, divided into three different categories: non-SARS-CoV-2 control peptides (Control), and SARS-CoV-2 Spike(S) and Nucleocapsid (N) peptides. All three of these comparisons are statistically significant (t-test, p<0.05). Individual data points are shown as circles, the limits of the boxes correspond to the 1st and 3rd quartiles, the black line inside each box corresponds to the median and the whiskers extend to points that lie within 1.5 interquartile ranges of the 1st and 3rd quartiles.

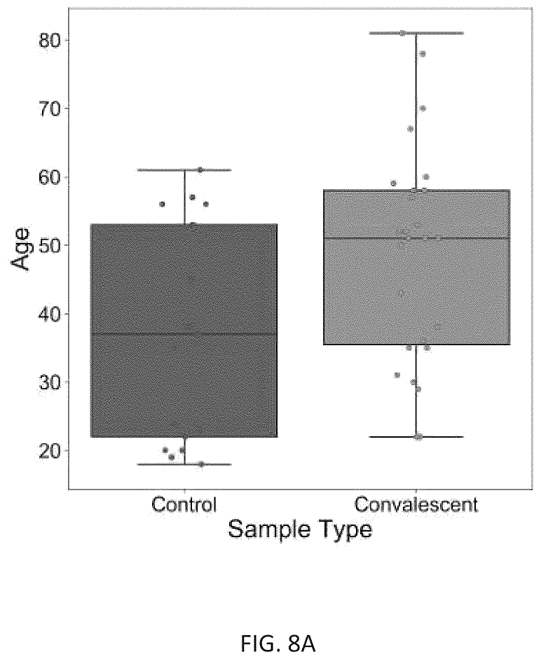

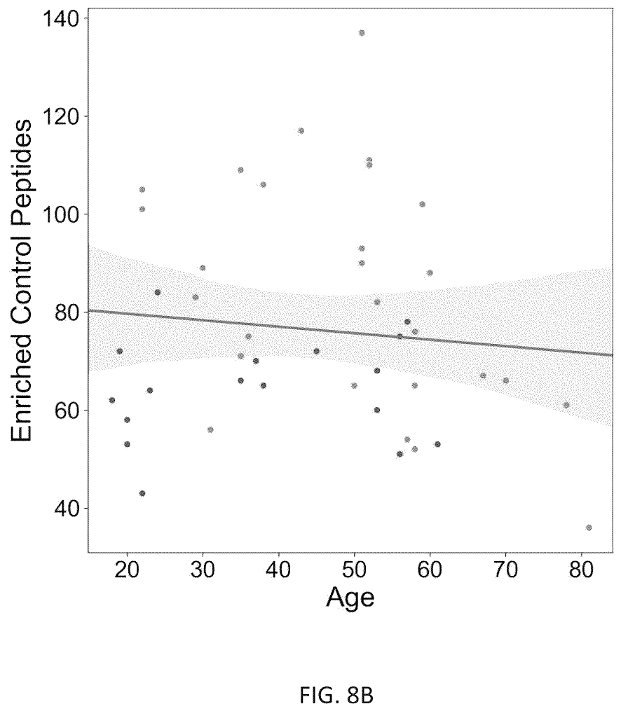

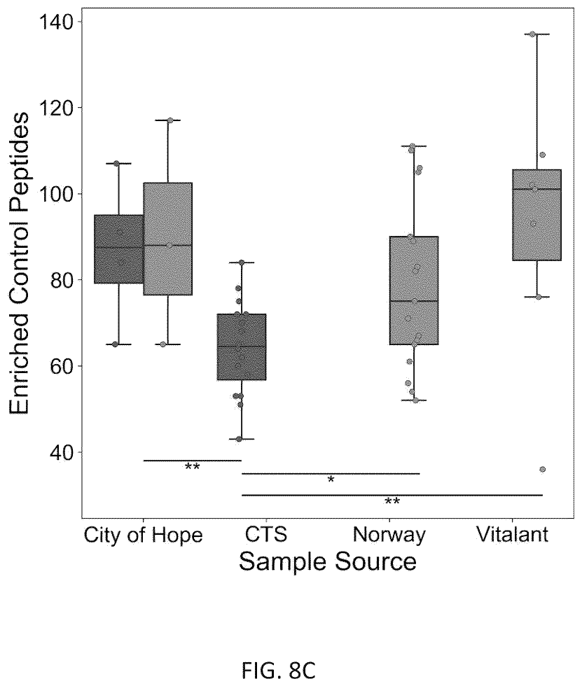

Unexpectedly, we observed a small, but significant increase in the average number of enriched control peptides between convalescent and control donors, which involved peptides designed from a wide variety of virus species (t-test, p=0.01, 1.2 fold difference). However, this difference was small compared to the difference in the number of enriched SARS-CoV-2 peptides (56-fold, p=2e−5). There was a significant difference in age between our convalescent and negative control donors, with our negative control donors being slightly younger on average than our COVID-19 convalescent donors (FIG. 8A, 1.3-fold, p=0.015). FIG. 8A shows boxplots depicting donor age distributions for negative control and convalescent serum/plasma samples. The means of these distributions are significantly different based on a t-test (p=0.022).

However, within our sample set, we did not observe a correlation between donor age and the number of enriched control peptides (FIG. 8B). FIG. 8B shows a scatterplot comparing donor age (x-axis) and the number of enriched SCV2 library control peptides (y-axis). Each circle represents a single serum/plasma sample. Grey line and band represent the best fit linear regression line and 95% confidence interval, respectively, as estimated by the Seaborn regplot( ) function.

In contrast, we did observe several significant pairwise differences in the number of enriched control peptides when we compared samples obtained from different sources (FIG. 8C). FIG. 8C shows boxplots depicting SCV2 library enriched control peptide distributions for each sample source and type. The mean for the negative control samples from Creative Testing Solutions (CTS) is significantly lower than that for the samples from the three other sources based on t-tests. Significantly different pairs are indicated with horizontal lines: *<0.05, **<0.01. For all boxplots, individual data points are shown as circles, the limits of the colored boxes correspond to the 1st and 3rd quartiles, the black line inside each box corresponds to the median and the whiskers extend to points that lie within 1.5 interquartile ranges of the 1st and 3rd quartiles. In FIGS. 8A-8C, blue is used to indicate negative control samples and orange for convalescent samples.

Specifically, we observed significantly fewer enriched control peptides in our pre-pandemic negative controls (1.2-1.5-fold, p=0.001-0.01). Therefore, this difference can likely be attributed to subtle differences in patient characteristics, sample collection, handling and/or storage among our different donor cohorts. We do not expect this difference to impact the conclusions discussed herein.

In total, we identified IgG reactivity (i.e., peptide enrichment) against 142 and 8 SARS-CoV-2 peptides in convalescent and negative control samples, respectively. All peptides that were enriched in any of the convalescent samples, regardless of the virus from which they were designed, are shown in TABLE 3. TABLE 3 shows the 142 SARS-CoV-2 enriched peptides, including SEQ ID NOS: 1-4 and 275-412, along with the other enriched peptides, including SEQ ID NOS: 5-274 and 413-420.

TABLE 3
Peptides enriched in convalescent samples.

| | | | Pro- | | Pro- | |
|---|---|---|---|---|---|---|
| | | COVID-19 | portion | Negative | portion | SEQ |
| | | Conv | COVID-19 | Control | Negative | ID |
| Peptide sequence | Organism | Samples | Conv | Samples | Control | NO: |
| SLIKRQGNRVIDAEPREIPLEYADDLLEAM | Aichivirus A | 17 | 0.63 | 6 | 0.286 | 5 |
| EACWKCSQDKPRRKYNTVPPEEWLYDSDVQ | Aichivirus A | 5 | 0.185 | 0 | 0 | 6 |
| ALPGIRRQGLLTLSADTETNQTLNKITESV | Aichivirus A | 7 | 0.259 | 5 | 0.238 | 7 |
| KFFDKLALLSLPGAYQAKTPEERALAGALT | Aichivirus A | 4 | 0.148 | 1 | 0.048 | 8 |
| TQSGNAAILTGSTAPSFLAYPTATPVPLPN | Aichivirus A | 6 | 0.222 | 2 | 0.09:5 | 9 |
| GSSNKVGSRFSKWWEPAAARALERATDSAI | Aichivirus A | 4 | 0.148 | 0 | 0 | 10 |
| MATQGPRVNWGDEPSKRRGRSNSRGRKSSD | Alphacoronavirus 1 | 1 | 0.037 | 0 | 0 | 11 |
| MSLWRPSEATVYLPPAPVSKVYSTDEYVTR | Alphapapillomavirus 9 | 1 | 0.037 | 0 | 0 | 12 |
| TTGYRFTNFEPFTVNSVNDSLEPVGGLYEI | Betacoronavirus 1 | 13 | 0.481 | 10 | 0.476 | 13 |
| APDVMLNISTPKLPDFKEELDQWFKNQTSV | Betacoronavirus 1 | 21 | 0.778 | 1 | 0.048 | 14 |
| KWADQSDQFRNVQTRGRRAQPKQTVTSQQP | Betacoronavirus 1 | 1 | 0.037 | 2 | 0.095 | 15 |
| AANTAASAHSLGTGRVPALQAAETGASSNS | Enterovirus A | 12 | 0.444 | 11 | 0.524 | 16 |
| VSDYIKGLGDAFGVGFTDAVSREVEALKNH | Enterovirus A | 18 | 0.667 | 15 | 0.714 | 17 |
| DSIADMIDQAVNNQVNRSLTAMQVLPTAAN | Enterovirus A | 10 | 0.37 | 8 | 0.381 | 18 |
| DMANAAKGFEWISNKISKFIDWIKEKIIPA | Enterovirus A | 9 | 0.333 | 9 | 0.429 | 19 |
| PPKFRPVRISLDEKPAPDAISDLLASVDSE | Enterovirus A | 10 | 0.37 | 6 | 0.286 | 20 |
| NLEAIDLHTSAGYPYSALGIKKRDILDPTT | Enterovirus A | 2 | 0.074 | 1 | 0.048 | 21 |
| DKRLEVDFETALFSKYIGNKIYEPDEYMIQ | Enterovirus A | 6 | 0.222 | 6 | 0.286 | 22 |
| TAEYQNDPITNAVENAVSALADTTISRVTA | Enterovirus A | 2 | 0.074 | 3 | 0.143 | 23 |
| VSKMKFYMDKYGLDLPYSTYVKDELRSMDK | Enterovirus A | 3 | 0.111 | 7 | 0.333 | 24 |
| FTNINYYKDSYAASAAKHDFTQDPGKFTQP | Enterovirus A | 2 | 0.074 | 4 | 0.19 | 25 |
| SNKETGRLSINGPTRTKLEPSAFYDVFEGS | Enterovirus A | 2 | 0.074 | 3 | 0.143 | 26 |
| QQVPALTAVETGHTSQVAPSDTIQTRHVHN | Enterovirus B | 21 | 0.778 | 15 | 0.714 | 27 |
| VEGAIGRVADTIRSGPSNSEAVPALTAAET | Enterovirus B | 15 | 0.556 | 16 | 0.762 | 28 |
| KDAGYPVINAPSKTKLEPSVFHQVFEGNKE | Enterovirus B | 17 | 0.63 | 13 | 0.619 | 29 |
| NRQDFTQDPSRFTEPVQDVLIKTLPALNSP | Enterovirus B | 6 | 0.222 | 6 | 0.286 | 30 |
| ALYQNDPESALNRAVGRVADTVASGPVNTE | Enterovirus B | 8 | 0.296 | 6 | 0.786 | 31 |
| ASEVTVSDTQPSGPSNSVSVPMLTAAETGH | Enterovirus B | 5 | 0.185 | 7 | 0.333 | 32 |
| DVVEAIESAVARVADTISSGPTNSQAVPAL | Enterovirus B | 8 | 0.296 | 8 | 0.381 | 33 |
| SPFVRQTVFYQNDVQNAIDRAMTRVADTMP | Enterovirus B | 2 | 0.074 | 3 | 0.143 | 34 |
| NEPSNAIERAMVRVADTMASGPANSEQIPA | Enterovirus B | 7 | 0.259 | 5 | 0.238 | 35 |
| SQDNKLQGDVEEAIERAVVHVADTMRSGPS | Enterovirus B | 5 | 0.185 | 3 | 0.143 | 36 |
| ITDYIQSLGNAFGAGFTETISSKAKEVQDM | Enterovirus D | 70 | 0.741 | 13 | 0.619 | 37 |
| IIKTATDTVKSEINAELGVVPSLNAVETGA | Enterovirus D | 13 | 0.481 | 9 | 0.429 | 38 |
| KDKRLKVDFEEAIFSKYVGNKTMLMDEYME | Enterovirus D | 7 | 0.259 | 8 | 0.381 | 39 |
| GVIPSLNAAETGATSNTTPEEAIQTRAVIN | Enterovirus D | 3 | 0.111 | 0 | 0 | 40 |
| RDIFNRQTRDTTEMTKMLDKYGVDLPFVTF | Enterovirus D | 1 | 0.037 | 5 | 0.238 | 41 |
| RGKERAPNALNAIIGNRDSVKAMPHNIVTT | Enterovirus D | 3 | 0.111 | 2 | 0.095 | 42 |
| LFQGPPQFREIKISVSPETPAPDAINDLLR | Enterovirus D | 1 | 0.037 | 0 | 0 | 43 |
| ANYKGKEKTPNALNALIGNRDNVKTMTHNI | Enterovirus D | 1 | 0.037 | 0 | 0 | 44 |
| RRYFTAEQGKIEYIEKSKEAGYPIINAPTQ | Enterovirus E | 7 | 0.259 | 4 | 0.19 | 45 |
| KFTEAVNAFKGLDWIAAKFSKFLDWIKSKI | Enterovirus E | 1 | 0.037 | 0 | 0 | 46 |
| SVDSEDVREYCRQKGWIVQEKITKESLERN | Enterovirus E | 3 | 0.111 | 3 | 0.143 | 47 |
| WMRRRTQKAPKRIRLPHIREDDQPSAHQPL | Human alphaherpesvirus | 14 | 0.519 | 10 | 0.476 | 48 |
| | 1 | | | | | |
| GDFDEAKLAEAREMIRYMALVSAMEHTEHK | Human alphaherpesvirus | 16 | 0.593 | 10 | 0.476 | 49 |
| | 1 | | | | | |
| PLDGCGPLHPSWVSLMPPKQVPETVVDAAC | Human alphaherpesvirus | 13 | 0.481 | 9 | 0.429 | 50 |
| | 1 | | | | | |
| AWGQVHDWTEQTDPWFLDGLGMDRMYWRDT | Human alphaherpesvirus | 11 | 0.407 | 10 | 0.476 | 51 |
| | 1 | | | | | |
| SGTPAPAPPGDGSYLWIPASHYNQLVAGHA | Human alphaherpesvirus | 16 | 0.593 | 10 | 0.476 | 52 |
| | 1 | | | | | |
| PSTQTRAPLPTEPAFPPAATGSQPEASNAE | Human alphaherpesvirus | 13 | 0.481 | 8 | 0.381 | 53 |
| | 1 | | | | | |
| HRPAPGSPPGIPEYAEDPYAISYGGQLDRY | Human alphaherpesvirus | 12 | 0.444 | 9 | 0.429 | 54 |
| | 1 | | | | | |
| AAPASPYIEAENPLYDWGGSALFSPPGRTG | Human alphaherpesvirus | 13 | 0.481 | 10 | 0.476 | 55 |
| | 1 | | | | | |
| WHIPSIQDAATPYHPPATPNNMGLIAGAVG | Human alphaherpesvirus | 12 | 0.444 | 8 | 0.381 | 56 |
| | 1 | | | | | |
| RSMAESDVVMEDVAIAERALGLSAFGVAGG | Human alphaherpesvirus | 12 | 0.444 | 6 | 0.286 | 57 |
| | 1 | | | | | |
| ASGKGPTYIRVADSELYADWSSDSEGERDQ | Human alphaherpesvirus | 11 | 0.407 | 7 | 0.333 | 58 |
| | 1 | | | | | |
| FWVRRRAQMAPKRLRLPHIRDDDAPPSHQP | Human alphaherpesvirus | 7 | 0.259 | 3 | 0.143 | 59 |
| | 2 | | | | | |
| EAYYSESEDEAANDFLVRMGRQQSVLRRRR | Human alphaherpesvirus | 14 | 0.519 | 3 | 0.143 | 60 |
| | 2 | | | | | |
| DQTLQLHREGVSTQDPRFVGAFMAAKAAHL | Human alphaherpesvirus | 8 | 0.296 | 6 | 0.286 | 61 |
| | 2 | | | | | |
| PSEAVRPSRIPRAPRVPREPRVPREPREPR | Human alphaherpesvirus | 9 | 0.333 | 6 | 0.286 | 62 |
| | 2 | | | | | |
| ASGPHETITALVGAVTSLQQELAHMRARTN | Human alphaherpesvirus | 5 | 0.185 | 0 | 0 | 63 |
| | 2 | | | | | |
| RRRHEVEQPEYDCGRDEPDRDFPYYPGEAR | Human alphaherpesvirus | 8 | 0.296 | 5 | 0.238 | 64 |
| | 2 | | | | | |
| KKGTSALLSSKVTNMVLRKRNKARYSPLHN | Human alphaherpesvirus | 5 | 0.185 | 2 | 0.095 | 65 |
| | 2 | | | | | |
| AGVYDAVRTWGPDAEAEPDQMENTYLLPDD | Human alphaherpesvirus | 5 | 0.185 | 1 | 0.048 | 66 |
| | 2 | | | | | |
| APHAWGMLNDMQWLASSDSEEETEVGISDD | Human alphaherpesvirus | 5 | 0.185 | 1 | 0.048 | 67 |
| | 2 | | | | | |
| DRDSSMSLADFHGEEFEKLYEHLEAMGEGE | Human alphaherpesvirus | 4 | 0.148 | 4 | 0.19 | 68 |
| | 2 | | | | | |
| DPIYDEVAPDHEAELYARVQRPGPVPDAEP | Human alphaherpesvirus | 1 | 0.037 | 1 | 0.048 | 69 |
| | 2 | | | | | |
| LENAHEHHGVYNQGRGIDSGERLMQPTQMS | Human alphaherpesvirus | 6 | 0.222 | 5 | 0.238 | 70 |
| | 3 | | | | | |
| AVAPTSAATRKPDPAVAPTSAASRKPDPAV | Human alphaherpesvirus | 8 | 0;296 | 4 | 0.19 | 71 |
| | 3 | | | | | |
| KGLKQLPEGMDPFAEKPNATDTPIEEIGDS | Human alphaherpesvirus | 10 | 0.37 | 2 | 0.095 | 72 |
| | 3 | | | | | |
| DKFREAQEMIKYMTLVSAAERQESKARKKN | Human alphaherpesvirus | 12 | 0.444 | 3 | 0.143 | 73 |
| | 3 | | | | | |
| HAESSWVNRGESSRKAYDHNSPYPNPRNDY | Human alphaherpesvirus | 5 | 0.185 | 4 | 0.19 | 74 |
| | 3 | | | | | |
| VVTTETKSVVKEGIENHVYPTDMSTLPEKS | Human alphaherpesvirus | 4 | 0.148 | 2 | 0.095 | 75 |
| | 3 | | | | | |
| HQPNDSSGSEDDFEDIDEVVAAFREARLRH | Human alphaherpesvirus | 3 | 0.111 | 0 | 0 | 76 |
| | 3 | | | | | |
| EPMYAQVRKPKSRTDTQTTGRITNRSRARS | Human alphaherpesvirus | 1 | 0.037 | 0 | 0 | 77 |
| | 3 | | | | | |
| IPADEEAPTTPEDPRHPLHAHQLVPNSLNV | Human alphaherpesvirus | 6 | 0.222 | 2 | 0.095 | 78 |
| | 3 | | | | | |
| APTPTFAGTQTPVNGNSPWAPTAPLPGDMN | Human betaherpesvirus 5 | 14 | 0.519 | 8 | 0.381 | 79 |
| PANWPRERAWALKNPHLAYNPFRMPTTSTA | Human betaherpesvirus 5 | 14 | 0.519 | 5 | 0.238 | 80 |
| STPRAAVTQTASQNAADEVWALRDQTAESP | Human betaherpesvirus 5 | 4 | 0.148 | 6 | 0.286 | 81 |
| VKYQALATASGEEVAALSHHDSLESRRLRE | Human betaherpesvirus 5 | 5 | 0.185 | 7 | 0.095 | 82 |
| ASEALDPHAFHLLLNTYGRPIRLLRENTTQ | Human betaherpesvirus 5 | 5 | 0.185 | 4 | 0.19 | 83 |
| PSLKPTLGGKAVVGRPPSVPVSGSAPGRLS | Human betaherpesvirus 5 | 10 | 0.37 | 3 | 0.143 | 84 |
| QQNGTDSLDGRTGTQDKGQKPNLLDRLRIR | Human betaherpesvirus 5 | 7 | 0.259 | 7 | 0.095 | 85 |
| EQPTETPPEDLDTLSLAIEAAIQDLRNKSQ | Human betaherpesvirus 5 | 10 | 0.37 | 4 | 0.19 | 86 |
| ASTTPTYPAVTTVYPPSSTAKSSVSNAPPV | Human betaherpesvirus 5 | 6 | 0.222 | 1 | 0.048 | 87 |
| ALYMGSRRIPRKPRYTRLPKHDPDEFWTKT | Human betaherpesvirus 5 | 8 | 0.296 | 2 | 0.095 | 88 |
| GRGSPLTIESHLSDNEEDPIRYVVSVYDEL | Human betaherpesvirus 5 | 6 | 0.222 | 2 | 0.095 | 89 |
| SDPLEAFKTVNRHNWSDEQREHFYDLRNLY | Human betaherpesvirus 6 | 17 | 0.63 | 12 | 0.571 | 90 |
| RKQKKLDLLGSWTKEKNDKAIVHSREVTGD | Human betaherpesvirus 6 | 13 | 0.481 | 12 | 0.571 | 91 |
| NTAANADVFDPVHRLVSEQTGTPFVLNNSD | Human betaherresvirus 6 | 4 | 0.148 | 6 | 0.286 | 92 |
| PESDSVDNAGGKILIKKETLGGRDVRATTP | Human betaherpesvirus 6 | 9 | 0.333 | 6 | 0.786 | 93 |
| KLPGNGEREIDLALQKVKAGERETSDFKVG | Human betaherpesvirus 6 | 8 | 0.296 | 2 | 0.095 | 94 |
| GDEYSQEDALKMLKAIKSLDESYRRKPSSS | Human betaherpesvirus 6 | 9 | 0.333 | 4 | 0.19 | 95 |
| EISDNIYSSPKNSIYLKSKSQQSTTKFTDT | Human betaherpesvirus 6 | 8 | 0.296 | 1 | 0.048 | 96 |
| TTHSTETGVSPHNVSLIKDLRDKDGFRKQK | Human betaherpesvirus 6 | 3 | 0.111 | 3 | 0.143 | 97 |
| VQSPFRLPNADLSRDLDSASFKDALDLKLP | Human betaherpesvirus 6 | 3 | 0.111 | 3 | 0.143 | 98 |
| KEKRKVEDIDKKKEDEKRKQEEKKRNDEDK | Human betaherpesvirus 6 | 4 | 0.148 | 7 | 0.095 | 99 |
| LKNLLNSRKRDPLFQNFSFTEKMQPVRSPF | Human betaherpesvirus 7 | 7 | 0.259 | 1 | 0.048 | 100 |
| QLVKDVKWTPSSSLLDLSRRNDLLQKELFE | Human betaherpesvirus 7 | 5 | 0.185 | 0 | 0 | 101 |
| NKIDYHSTFFLPENEVNRQNGVQSRDQLSK | Human betaherpesvirus 7 | 6 | 0.222 | 4 | 0.19 | 107 |
| DLIDLENSVQKDDDIVNKLVSHLTHSEEDV | Human betaherpesvirus 7 | 5 | 0.185 | 1 | 0.048 | 103 |
| QDFDSGSLLTGKETQNTIFGASKAQENGDK | Human betaherpesvirus 7 | 4 | 0.148 | 0 | 0 | 104 |
| ATPIERSSRSASIISGESVPGFFNDQERLS | Human betaherpesvirus 7 | 3 | 0.111 | 1 | 0.048 | 105 |
| RRKRELETNKDIVYVQLQYLYDTLKDYINT | Human betaherpesvirus 7 | 1 | 0.037 | 0 | 0 | 106 |
| PVLNISRPGSTTPSGNSARYGNNTPRSITP | Human betaherpesvirus 7 | 3 | 0.111 | 0 | 0 | 107 |
| NSVLQATQSVQAQVKEPLDSSPPYLKTNKD | Human betaherpesvirus 7 | 1 | 0.037 | 2 | 0.095 | 108 |
| KRRKEIVHENLQSFDDEHNEMSLPPQDQKS | Human betaherpesvirus 7 | 1 | 0.037 | 0 | 0 | 109 |
| YTVPDLVVEQYNQTILNLTSEISTRENKSA | Human coronavirus 229E | 2 | 0.074 | 0 | 0 | 110 |
| SDRNHNSQDDIMKAVAAALKSLGFDKPQEK | Human coronavirus 229E | 4 | 0.148 | 3 | 0.143 | 111 |
| EFNPSQTSPATVEPVRDEVSIETDIIDEVN | Human coronavirus 229E | 7 | 0.259 | 4 | 0.19 | 112 |
| GAMLSENFTSYGFSNVVEMPKFFYASNGTY | Human coronavirus 229E | 5 | 0.185 | 5 | 0.238 | 113 |
| SNQDDIMAAVAAALEKLGFERPNDASQPQK | Human coronavirus 229E | 1 | 0.037 | 0 | 0 | 114 |
| DYALPSSRRKRRGISSPYRFVTFEPENVSF | Human coronavirus HKU1 | 10 | 0.37 | 2 | 0.095 | 115 |
| KPDMADEIASLVLAKLGKDSKPQQVTKQNA | Human coronavirus HKU1 | 5 | 0.185 | 3 | 0.143 | 116 |
| FGLQDGFYSANFLDDNVLPETYVALPTYYQ | Human coronavirus NL63 | 7 | 0.259 | 6 | 0.286 | 117 |
| NTVLNASIPESKPLADDDSAIIEIVNEVLH | Human coronavirus NL63 | 2 | 0.074 | 3 | 0.143 | 118 |
| ERWRMRRGQRVDLPPKVHFYYLGTGPHKDL | Human coronavirus NL63 | 1 | 0.037 | 0 | 0 | 119 |
| VSNGGNNCTTAVMTYSNFGICADGSLIPVR | Human coronavirus NL63 | 1 | 0.037 | 1 | 0.048 | 120 |
| PGRRPFFHPVGQADYFEYHQEGGPDGEPDM | Human | 71 | 0.778 | 18 | 0.857 | 121 |
| | gammaherpesvirus 4 | | | | | |
| SPSLPSSKKGADEFEAWLEAQDANFEDVQR | Human | 20 | 0.741 | 17 | 0.81 | 122 |
| | gammaherpesvirus 4 | | | | | |
| MMDPNSTSEDVKFTPDPYQVPFVQAFDQAT | Human | 24 | 0.889 | 12 | 0.571 | 123 |
| | gammaherpesvirus 4 | | | | | |
| QHASGEGPGINPISKTELQAIMLALHEQNQ | Human | 16 | 0.593 | Q | 0.429 | 124 |
| | gammaherpesvirus 4 | | | | | |
| EQEYGDKEVKLPHWTPTLHTFQVPKNYTKA | Human | 18 | 0.667 | 5 | 0.238 | 125 |
| | gammaherpesvirus 4 | | | | | |
| GPRHRDGVRRPQKRPSCIGCKGAHGGTGTG | Human | 11 | 0.407 | 13 | 0.619 | 126 |
| | gammaherpesvirus 4 | | | | | |
| QPMEGPLVPEQWMFPGAALSQRVRPGVAQS | Human | 16 | 0.593 | 11 | 0.524 | 127 |
| | gammaherpesvirus 4 | | | | | |
| MHPLTHQSIPNDPDSPEPRSPTVFYNIPPM | Human | 21 | 0.778 | 9 | 0.429 | 128 |
| | gammaherpesvirus 4 | | | | | |
| LPPRVRGGGRVSAAAITWVPKPNVEVWPVD | Human | 15 | 0.556 | 10 | 0.476 | 129 |
| | gammaherpesvirus 4 | | | | | |
| RKPGGPWRPEPNTSSPSMPELSPVLGLHQG | Human | 13 | 0.481 | 6 | 0.286 | 130 |
| | gammaherpesvirus 4 | | | | | |
| SPIHEPESHNSPEAPILFPDDWYPPSIDPA | Human | 20 | 0.741 | 12 | 0.571 | 131 |
| | gammaherpesvirus 4 | | | | | |
| LAAPRRGNVYWVRDAVTGTRVPVRTRPPHP | Human mastadenovirus A | 17 | 0.63 | 12 | 0.571 | 132 |
| IESINDKMSRWKTYAQERHEWEERQPKPVP | Human mastadenovirus A | 18 | 0.667 | 10 | 0.476 | 133 |
| EENDDFNPVYPFDPYDTAHVPFVTPPFTSS | Human mastadenovirus A | 5 | 0.185 | 2 | 0.095 | 134 |
| MSKDIPTPYMWSFQPQMGLAAGAAQDYSSK | Human mastadenovirus A | 11 | 0.407 | 3 | 0.143 | 135 |
| PPGFYTGEFDLPEGNDGFLWDDVTDSLFSP | Human mastadenovirus A | 1 | 0.037 | 0 | 0 | 136 |
| VLEYMKVDPNIQPDVKIRPIKKVAPGLGVQ | Human mastadenovirus A | 7 | 0.259 | 1 | 0.048 | 137 |
| EAPPPSYETVMAAAQTSALEAPYVPPRYLA | Human mastadenovirus A | 1 | 0.037 | 0 | 0 | 138 |
| GHYRAPWGAHTRGRTGRTTVDDVIDSVVAD | Human mastadenovirus A | 1 | 0.037 | 0 | 0 | 139 |
| LKDQNFQQKVVDGLASGINGVVDIANQAVQ | Human mastadenovirus A | 1 | 0.037 | 3 | 0.143 | 140 |
| NNPQVVFYTEDVNLEMPDTHLVFKPTVTDG | Human mastadenovirus A | 1 | 0.037 | 0 | 0 | 141 |
| RASRRRQRHDRQRGLVWEDEDSADDSSVLD | Human mastadenovirus B | 13 | 0.481 | 6 | 0.286 | 142 |
| WPAALVYQESPAPTTVLLPRDAQAEVQMTN | Human mastadenovirus B | 7 | 0.259 | 4 | 0.19 | 143 |
| TLVTRADEPPSYEEAVKLGMPTTRPVAHMA | Human mastadenovirus B | 14 | 0.519 | 9 | 0.429 | 144 |
| SFNPVYPYEDESTSQHPFINPGFISPNGFT | Human mastadenovirus B | 6 | 0.222 | 7 | 0.333 | 145 |
| TVDDTDGTLQENIGTTTPLVKTGESIGLSL | Human mastadenovirus B | 4 | 0.148 | 1 | 0.048 | 146 |
| PPLQPFDPPTLHDLYDLEVDGPDDPNEEAV | Human mastadenovirus B | 3 | 0.111 | 1 | 0.048 | 147 |
| EEIEADVEQDPGYVTPAEHEEELKRFLDRE | Human mastadenovirus B | 1 | 0.037 | 0 | 0 | 148 |
| DMNDHAIRGDTFATRAEEKRAEAEAAAEAA | Human mastadenovirus C | 25 | 0.926 | 19 | 0.905 | 149 |
| AATQKQRRPDSKTLTKPKKSTAAAAAGGGA | Human mastadenovirus C | 9 | 0.333 | 6 | 0.286 | 150 |
| TTDYRNTTATGLTSALNLPQVHAFVNDWAS | Human mastadenovirus C | 10 | 0.37 | 7 | 0.333 | 151 |
| PSAPAVSTVDEAIESVVQGARHYANLKNRR | Human mastadenovirus C | 11 | 0.407 | 2 | 0.095 | 152 |
| ARNYTPTPPPVSTVDAAIQTVVRGARRYAK | Human mastadenovirus C | 10 | 0.37 | 4 | 0.19 | 153 |
| EVLDEEEEMMEDWDSLDEEASEAEEVSDET | Human mastadenovirus C | 6 | 0.222 | 5 | 0.238 | 154 |
| ARRTGRRAAMRAARRLAAGIVTVPPRSRRR | Human mastadenovirus C | 5 | 0.185 | 1 | 0.048 | 155 |
| IAPMATGVLGHHTPVTLDLPPPADTQQKPV | Human mastadenovirus C | 5 | 0.185 | 2 | 0.095 | 156 |
| SYESVVSAASVAAALGSPFDAPLDPPFVPP | Human mastadenovirus C | 11 | 0.407 | 8 | 0.381 | 157 |
| TTRPRLLGEEEYLNNSLLQPQREKNLPPAF | Human mastadenovirus C | 6 | 0.222 | 2 | 0.095 | 158 |
| PEDARPVVSDEMLARWLGTRDPQALEQRRK | Human mastadenovirus D | 2 | 0.074 | 1 | 0.048 | 159 |
| MTKRLRVEDDFNPIYPYGYARNQNIPFLTP | Human mastadenovirus D | 1 | 0.037 | 1 | 0.048 | 160 |
| ESYKNEIKKLTYKNNKTTFEDSGNYEHQKL | Human mastadenovirus D | 1 | 0.037 | 1 | 0.048 | 161 |
| EKINQSLTFIRKSDELLHNVNVGKSTTNIM | Human | 10 | 0.37 | 6 | 0.286 | 162 |
| | orthopneumovirus | | | | | |
| FEAFNFVPCSICSGNPTCWAICKRIPNKKP | Human | 17 | 0.63 | 10 | 0.476 | 163 |
| | orthopneumovirus | | | | | |
| NYQRKPLVSFKEDPTPSDNPFSKEYKETIE | Human | 2 | 0.074 | 2 | 0.095 | 164 |
| | orthopneumovirus | | | | | |
| NKLGEKEKEKDKIKSNNEQDENNSVITTII | Human | 6 | 0.222 | 3 | 0.143 | 165 |
| | orthopneumovirus | | | | | |
| STYMLTNSELLSLINDMPITNDQKKLMSSN | Human | 14 | 0.519 | 6 | 0.286 | 166 |
| | orthopneumovirus | | | | | |
| TNKPSTKPHPKIPPKKPKDDYHFEVFNFVP | Human | 10 | 0.37 | 2 | 0.095 | 167 |
| | orthopneumovirus | | | | | |
| CSICGNNQLCKSICKTIPGNKPKKKPTIKP | Human | 8 | 0.296 | 1 | 0.048 | 168 |
| | orthopneumovirus | | | | | |
| QAITCQKPTPEKEKPDVYKNLSFWEVNLKE | Human papillomavirus | 1 | 0.037 | 1 | 0.048 | 169 |
| PPAEKKDPYADLTFWEVDLKERFSLELDQF | Human papillomavirus | 1 | 0.037 | 1 | 0.048 | 170 |
| AGKEGDSIPMEGTDYYIARQDSKLASHIYY | Human papillomavirus | 1 | 0.037 | 1 | 0.048 | 171 |
| AHQPDFGTWNSSEVPTYGTEEWESWWSSFN | Human polvomavirus 1 | 6 | 0.222 | 3 | 0.143 | 172 |
| RVFDGTERLPGDPDMIRYIDKQGQLQTKML | Human polyomavirus 1 | 2 | 0.074 | 1 | 0.048 | 173 |
| LSDEIQRLLRDLEYGFRATLASIGESDPVN | Human polyomavirus 3 | 6 | 0.222 | 5 | 0.238 | 174 |
| NIWQSSQIPTYGTPDWDEWWSQFNTYWEEE | Human polvomavirus 3 | 3 | 0.111 | 0 | 0 | 175 |
| LQSVHKPIHAPYSGMALVPIPEYQLETGIP | Human polyomavirus 3 | 1 | 0.037 | 0 | 0 | 176 |
| DEDITDIENKIARRLADRKQRLSQANNKRD | Human respirovirus 1 | 13 | 0.481 | 4 | 0.19 | 177 |
| NETTDYSSEGIEDLVFDILDLKGKTKSHRY | Human respirovirus 1 | 1 | 0.037 | 0 | 0 | 178 |
| GAIEVAIDHTDITFGAEDTADRDNKNWAND | Human respirovirus 1 | 1 | 0.037 | 0 | 0 | 179 |
| KKQGSQPPTNPTNRTNQDEIDDLFNAFGSN | Human respirovirus 3 | 19 | 0.704 | 12 | 0.571 | 180 |
| EQATESDNIKTEQQNIRDRLNKRLNDKKKQ | Human respirovirus 3 | 7 | 0.259 | 2 | 0.095 | 181 |
| IVLINSIKSERAHESLLQDINNEFMEVTEK | Human respirovirus 3 | 14 | 0.519 | 10 | 0.476 | 187 |
| ENRADQEQGGEPQSSIIQYAWAEGNRNDDR | Human respirovirus 3 | 5 | 0.185 | 1 | 0.048 | 183 |
| SIKSEKAHESLLRDINNEFIGITEKIQMAS | Human respirovirus 3 | 11 | 0.407 | 12 | 0.571 | 184 |
| STHQEDDKRIKKGGKGKDWFKKSKDTDNQT | Human respirovirus 3 | 1 | 0.037 | 0 | 0 | 185 |
| MESDAKNYQVMDSWEEESRDKSTNISSALN | Human respirovirus 3 | 2 | 0.074 | 1 | 0.048 | 186 |
| SLESIGTPDTRSISVVTAATPDDEEEILMK | Human respirovirus 3 | 2 | 0.074 | 0 | 0 | 187 |
| PQRTSGMSSEEFQHSMNQYIRAMHEQYRGS | Human rubulavirus 2 | 14 | 0.519 | s | 0.738 | 188 |
| MAEEPTYTTEQVDELIHAGLGTVDFFLSRP | Human rubulavirus 2 | 5 | 0.185 | 1 | 0.048 | 189 |
| QLPRGRQPISDPFAGANDREIGGQANDTPV | Human rubulavirus 2 | 2 | 0.074 | 1 | 0.048 | 190 |
| PSSSAGLKDDLLENLQAYQKRMGVQMQRFK | Influenza A virus | 8 | 0.296 | 1 | 0.048 | 191 |
| LATGMRNVPEKQTRGIFGAIAGFIENGWEG | Influenza A virus | 2 | 0.074 | 3 | 0.143 | 192 |
| SEQAAEAMEIASQARQMVQAMRTVGTHPSS | Influenza A virus | 3 | 0.111 | 1 | 0.048 | 193 |
| NPLIRHENRMVLASTTAKAMEQMAGSSEQA | Influenza A virus | 3 | 0.111 | 4 | 0.19 | 194 |
| RTLDYHDSNVKNLYEKVRSQLKNNAKEIGN | Influenza A virus | 1 | 0.037 | 0 | 0 | 195 |
| IWDPNGWTGTDNNFSIKQDIVGINEWSGYS | Influenza A virus | 2 | 0.074 | 0 | 0 | 196 |
| ATCEQIADAQHRSHRQMATTTNPLIKHENR | Influenza A virus | 2 | 0.074 | 0 | 0 | 197 |
| VETYVLSIIPSGPLKAEIAQKLEDVFAGKN | Influenza A virus | 3 | 0.111 | 3 | 0.143 | 198 |
| NPHRILDGIDCTLIDALLGDPHCDGFQNET | Influenza A virus | 1 | 0.037 | 0 | 0 | 199 |
| FAVERPIALSKQAVRKMLSMNIEGRDADVK | Influenza B virus | 70 | 0.741 | 11 | 0.524 | 200 |
| TTRPIIRPATLAPPSNKRTRNPSPERATTS | Influenza B virus | 17 | 0.63 | 11 | 0.524 | 201 |
| GTFNAGEFSLPTFDSLNITAASLNNDGLDN | Influenza B virus | 9 | 0.333 | 3 | 0.143 | 202 |
| RTRGKLCPECLNCTDLDVALGRPMCVGTTP | Influenza B virus | 7 | 0.259 | 5 | 0.738 | 203 |
| TNPIEIPIKQTIPNFFFGRDTAEDYDDLDY | Influenza B virus | 6 | 0.222 | 2 | 0.095 | 204 |
| SLSELEVKNLQRLSGAMDELHSEILELDEK | Influenza B virus | 1 | 0.037 | 0 | 0 | 205 |
| SNSPHVVKIATQGEVNVTGVIPLTTTPTKS | Influenza B virus | 5 | 0.185 | 1 | 0.048 | 206 |
| MSNMDIDGINTGTIDKAPEEITSGTSGTTR | Influenza B virus | 3 | 0.111 | 2 | 0.095 | 207 |
| NTAKTMNGMGKGEDVQKLAEELQSNIGVLR | Influenza B virus | 1 | 0.037 | 2 | 0.095 | 208 |
| GEADDHHGDQEMRELLSGLDYEARCISQSG | Influenza C virus | 2 | 0.074 | 2 | 0.095 | 209 |
| YLLPPKFGRCPLAAKEESIPKIPDGLLIPT | Influenza C virus | 2 | 0.074 | 1 | 0.048 | 210 |
| PTYGTDEWEQWWNAFNEENLFCSEEMPSSD | Macaca mulatta | 3 | 0.111 | 1 | 0.048 | 211 |
| | polyomavirus 1 | | | | | |
| VGVLDWLRNSDDDDDDEDGGEKNMEDSGHE | Macaca mulatta | 1 | 0.037 | 0 | 0 | 212 |
| | polyomavirus 1 | | | | | |
| RELVINTLVNQGISRDRATYIGMSAYPNVE | Mamastrovirus 1 | 3 | 0.111 | 1 | 0.048 | 213 |
| DIIDTSDEEYENETDRVTLLSTLVNQGMTM | Mamastrovirus 1 | 7 | 0.259 | 5 | 0.238 | 214 |
| FDEDPIATLHAVDAERKIRRAIFNALMEEG | Mamasfrovirus 1 | 3 | 0.111 | 1 | 0.048 | 215 |
| DQREKYRHVHEWYVDNLLNRHVLLPSGEVT | Mamastrovirus 1 | 3 | 0.111 | 4 | 0.19 | 216 |
| TSIPRSRASGHGYESDNTEYLDAPDSADQF | Mamastrovirus 1 | 1 | 0.037 | 0 | 0 | 217 |
| EEYGPTPWGPQAFIKSFDKFFYAEPIDFFS | Mamasfrovirus 1 | 1 | 0.037 | 0 | 0 | 218 |
| DRATLLSTLLNQGISVEPATRITNGAFPAR | Mamastrovirus 1 | 4 | 0.148 | 3 | 0.143 | 219 |
| DDEADRFDLHSSYGSEPEDDDENNRVTLLS | Mamastrovirus 1 | 1 | 0.037 | 0 | 0 | 220 |
| VTSDDTDYDTDTEDEDEFFGEDPIAALHAV | Mamasfrovirus 6 | 15 | 0.556 | 7 | 0.333 | 221 |
| SADGANEPVEMLIPVNEWNMKAQYGGNGTL | Mamastrovirus 6 | 2 | 0.074 | 1 | 0.048 | 772 |
| KGLSDEEYEEYKRVREERNGKYSIEEYLQD | Norwalk virus | 16 | 0.593 | 17 | 0.81 | 223 |
| DDFKLKGKLWADDDRSVDYNERLNFEAPPS | Norwalk virus | 4 | 0.148 | 3 | 0.143 | 224 |
| ISGLPDLTTVPQPDATNTAFSVPPLSLREN | Norwalk virus | 10 | 0.37 | 5 | 0.238 | 225 |
| APDIEKAKRDFPGQPDMWKDHFRPDFSHIK | Norwalk virus | 8 | 0.296 | 2 | 0.095 | 226 |
| TTGFFRPYQDWNKKPLPTVDDSKLKKVANI | Norwalk virus | 3 | 0.111 | 0 | 0 | 227 |
| NNYDPTEEIPAPLGTPDFVGKIQGVLTQFT | Norwalk virus | 1 | 0.037 | 1 | 0.048 | 228 |
| AYSVPPLSQREVGEAKEPLPGSILEMWDGE | Norwalk virus | 1 | 0.037 | 0 | 0 | 229 |
| DEEYDEYKKIREERGGKYSIQEYLEDRERF | Norwalk virus | 4 | 0.148 | 3 | 0.143 | 230 |
| MMMASKDAPTNMDGTSGAGQLVPEANTAEP | Norwalk virus | 1 | 0.037 | 3 | 0.143 | 231 |
| VVSYSVKDGVSGLPDLSTVRQPEESNTAFS | Norwalk virus | 1 | 0.037 | 0 | 0 | 232 |
| QDIHLIDDLGQTRKEKDIEMLCNCISSVPF | Parechovirus A | 2 | 0.074 | 0 | 0 | 233 |
| TTNLTQHPSAPTIPFTPDFRNVDNFHSMAY | Parechovirus A | 1 | 0.037 | 0 | 0 | 234 |
| SAPTMPFTPDFSNVDTFHSMAYDVTTGEKN | Parechovirus A | 2 | 0.074 | 2 | 0.095 | 235 |
| QIHKSPVYGAVEVKMGPAVLSKSDPRLEEP | Parechovirus A | 1 | 0.037 | 2 | 0.095 | 236 |
| VPITQNPVENYIDEVLNEVLVVPNIKESHP | Rhinovirus A | 27 | 1 | 21 | 1 | 237 |
| EQYIDGVLNEVLIVPNINESHPSTSNAAPA | Rhinovirus A | 25 | 0.926 | 20 | 0.952 | 238 |
| SAIFQGPISLGAPPPPAIADLLQSVRTPEV | Rhinovirus A | 76 | 0.963 | 20 | 0.952 | 239 |
| IFQGPIDMRNPPPPAITDLLQAVRTPEVIK | Rhinovirus A | 16 | 0.593 | 14 | 0.667 | 240 |
| ADEQGITDYIHTLGEAFGAGFVDNIKDQIQ | Rhinovirus A | 25 | 0.926 | 20 | 0.952 | 241 |
| VMEQNPVEKYTEAVLNEVLAVPNITPSNSQ | Rhinovirus A | 25 | 0.926 | 21 | 1 | 247 |
| NPSGEDMTLFCQMVSSVPFIPPMADLPDKG | Rhinovirus A | 26 | 0.963 | 21 | 1 | 243 |
| KLQPSVFYDVFPGSKEPAVLTSNDPRLEVD | Rhinovirus A | 12 | 0.444 | 17 | 0.81 | 244 |
| DFIADEQGLGDYITSLGRAFGTGFTDQISA | Rhinovirus A | 74 | 0.889 | 18 | 0.857 | 745 |
| PPPPAIMDLLKSVKNPEVIKYCEDNKWIIP | Rhinovirus A | 25 | 0.926 | 19 | 0.905 | 246 |
| TISQTDALTEGLGDELEEVIVEKTKQTLAS | Rhinovirus A | 24 | 0.889 | 19 | 0.905 | 247 |
| ECINDLLRSVDSEEVREYCKRKNWIIPQIP | Rhinovirus B | 70 | 0.741 | 12 | 0.571 | 248 |
| QSLHQETALTEGLEDELMEVIVDKTQQTLA | Rhinovirus B | 18 | 0.667 | 12 | 0.571 | 249 |
| VVPEHQLASHTQGNVSVKYKYTHPGEQGID | Rhinovirus B | 16 | 0.593 | 13 | 0.619 | 250 |
| QLASHDGGTVSVKYKFTHPGDQGIDLSTAE | Rhinovirus B | 18 | 0.667 | 13 | 0.619 | 251 |
| ISDLLKSVDSEEIREYCKQKNWLIPEIPTN | Rhinovirus B | 20 | 0.741 | 12 | 0.571 | 252 |
| HTQSVPALTANETGATLPTRPSDNVETRTT | Rhinovirus B | 19 | 0.704 | 5 | 0.238 | 253 |
| AATKMDFSQDPSKFTEPVKDVMIKTAPALN | Rhinovirus B | 17 | 0.63 | 11 | 0.524 | 254 |
| EDELEEVVIDKMKQVTASSQSGPKHTQKVP | Rhinovirus B | 13 | 0.481 | 5 | 0.238 | 255 |
| ELNMNPINTPTKSKLHPSVFYNVFPGDKEP | Rhinovirus B | 14 | 0.519 | 15 | 0.714 | 256 |
| LMKDTQTISQTEALTEGFEEELEEVVVDKM | Rhinovirus B | 12 | 0.444 | 9 | 0.429 | 257 |
| DVLEEVIVDKAKQTIASINSNSKYTQQVPT | Rhinovirus B | 10 | 0.37 | 2 | 0.095 | 258 |
| VEHNLTAIFQGLGDDTTPGFIIDLLSASKD | Rhinovirus C | 19 | 0.704 | 19 | 0.905 | 259 |
| NPVEDFIDTTLKEVLVVPDTHPSGPVHTTR | Rhinovirus C | 22 | 0.815 | 18 | 0.857 | 260 |
| AHQGLVSDYVNQLGAAFGDGFSSNIKDHLT | Rhinovirus C | 15 | 0.556 | 12 | 0.571 | 261 |
| AYIGGTNANVGYNHTHPGEIGHEIGRNTGR | Rhinovirus C | 12 | 0.444 | 12 | 0.571 | 262 |
| TNDLQNNDPIDTYVHDVLNEVVVVPDTKPS | Rhinovirus C | 18 | 0.667 | 16 | 0.762 | 263 |
| DTPMITQDKNTLQNPVEQFVDDVLEEVLVV | Rhinovirus C | 21 | 0.778 | 14 | 0.667 | 264 |
| IPEHQLAYAGGANASVGYKHTHPGENGHKI | Rhinovirus C | 14 | 0.519 | 9 | 0.429 | 265 |
| SSLSEHQGVTDYITQLGSAFGDGFTSSIKQ | Rhinovirus C | 20 | 0.741 | 13 | 0.619 | 266 |
| LRPYNNLAQTQGPISDYVTQLGNAFGNGFT | Rhinovirus C | 17 | 0.63 | 12 | 0.571 | 267 |
| DPVSDFIDATLQEVLVVPETKPSGPQHTTK | Rhinovirus C | 22 | 0.815 | 13 | 0.619 | 268 |
| PINTPSTTKLYPSVFYEIFPGEKEPAVLSD | Rhinovirus C | 6 | 0.222 | 13 | 0.619 | 269 |
| IDMSKEFNQKNIKTLDEWESGKNPYEPSEV | Rotavirus A | 6 | 0)22 | 2 | 0.095 | 270 |
| NERLQEKEIEKNADAIMENKNGNKKQQLSD | Rotavirus A | 4 | 0.148 | 5 | 0.238 | 271 |
| KEIENNTDVTMENKNKNKNKNNNRKQQLSD | Rotavirus A | 1 | 0.037 | 0 | 0 | 272 |
| KRRNVQQKDVEKEKQIEKMEEKEIKEVKEQ | Rotavirus C | 3 | 0.111 | 0 | 0 | 273 |
| IKTVPLENELKQKEKQRDNKEKNEKENKDE | Rotavirus H | 3 | 0.111 | 1 | 0.048 | 274 |
| FNGLTGTGVLTESNKKFLPFQQFGRDIADT | SARS-CoV-2 | 1 | 0.037 | 0 | 0 | 275 |
| GVLTESNKKFLPFQQFGRDIADTTDAVRDP | SARS-CoV-2 | 1 | 0.037 | 0 | 0 | 276 |
| KKFLPFQQFGRDIADTTDAVRDPQTLEILD | SARS-CoV-2 | 1 | 0.037 | 0 | 0 | 277 |
| KFLPFQQFGRDIADTTDAVRDPQTLEILDI | SARS-CoV-2 | 2 | 0.074 | 0 | 0 | 278 |
| FLPFQQFGRDIADTTDAVRDPQTLEILDIT | SARS-CoV-2 | 2 | 0.074 | 0 | 0 | 279 |
| LPFQQFGRDIADTTDAVRDPQTLEILDITP | SARS-CoV-2 | 3 | 0.111 | 0 | 0 | 280 |
| QILPDPSKPSKRSFIEDLLFNKVTLADAGF | SARS-CoV-2 | 8 | 0.296 | 0 | 0 | 281 |
| DPSKPSKRSFIEDLLFNKVTLADAGFIKQY | SARS-CoV-2 | 5 | 0.185 | 0 | 0 | 282 |
| PSKPSKRSFIEDLLFNKVTLADAGFIKQYG | SARS-CoV-2 | 6 | 0.222 | 0 | 0 | 283 |
| SKPSKRSFIEDLLFNKVTLADAGFIKQVGD | SARS-CoV-2 | 9 | 0.333 | 0 | 0 | 2 |
| SSVLNDILSRLDKVEAEVQIDRLITGRLQS | SARS-CoV-2 | 2 | 0.074 | 0 | 0 | 284 |
| NTVYDPLQPELDSTKEELDKYFKNHTSPDV | SARS-CoV-2 | 12 | 0.444 | 0 | 0 | 285 |
| TVYDPLQPELDSFKEELDKATKNHTSPDVD | SARS-CoV-2 | 10 | 0.37 | 0 | 0 | 286 |
| YDPLQPELDSFKEELDKYFKNHTSPDVDLG | SARS-CoV-2 | 6 | 0.222 | 0 | 0 | 287 |
| DPLQPELDSFKEELDKYFKNHTSPDVDLGD | SARS-CoV-2 | 7 | 0.259 | 0 | 0 | 288 |
| LQPELDSFKEELDKYFKNHTSPDVDLGDIS | SARS-CoV-2 | 8 | 0.296 | 0 | 0 | 289 |
| QPELDSFKEELDKYFKNHTSPDVDLGDISG | SARS-CoV-2 | 11 | 0.407 | 0 | 0 | 290 |
| PELDSFKEELDKYFKNHTSPDVDLGDISGI | SARS-CoV-2 | 9 | 0.333 | 0 | 0 | 291 |
| ELDSFKEELDKYFKNHTSPDVDLGDISGIN | SARS-CoV-2 | 8 | 0.296 | 0 | 0 | 292 |
| LDSFKEELDKYFKNHTSPDVDLGDISGINA | SARS-CoV-2 | 8 | 0.296 | 0 | 0 | 293 |
| DSFKEELDKYFKNHTSPDVDLGDISGINAS | SARS-CoV-2 | 11 | 0.407 | 0 | 0 | 294 |
| SFKEELDKYFKNHTSPDVDLGDISGINASV | SARS-CoV-2 | 12 | 0.444 | 0 | 0 | 1 |
| NTPKDHIGTRNPANNAAIVLQLPQGTTLPK | SARS-CoV-2 | 8 | 0.296 | 0 | 0 | 295 |
| TPKDHIGTRNPANNAAIVLQLPQGTTLPKG | SARS-CoV-2 | 8 | 0.296 | 0 | 0 | 296 |
| PKDHIGTRNPANNAAIVLQLPQGTTLPKGF | SARS-CoV-2 | 6 | 0.222 | 0 | 0 | 297 |
| KDHIGTRNPANNAAIVLQLPQGTTLPKGFY | SARS-CoV-2 | 7 | 0.259 | 0 | 0 | 298 |
| DHIGTRNPANNAAIVLQLPQGTTLPKGEYA | SARS-CoV-2 | 7 | 0.259 | 0 | 0 | 299 |
| HIGTRNPANNAAIVLQLPQGTTLPKGFYAE | SARS-CoV-2 | 7 | 0.259 | 0 | 0 | 300 |
| IGTRNPANNAAIVLQLPQGTTLPKGFYAEG | SARS-CoV-2 | 8 | 0.296 | 0 | 0 | 301 |
| GTRNPANNAAIVLQLPQGTTLPKGFYAEGS | SARS-CoV-2 | 7 | 0.259 | 0 | 0 | 302 |
| TRNPANNAAIVLQLPQGTTLPKGFYAEGSR | SARS-CoV-2 | 7 | 0.259 | 0 | 0 | 303 |
| RNPANNAAIVLQLPQGTTLPKGFYAEGSRG | SARS-CoV-2 | 8 | 0.296 | 0 | 0 | 304 |
| NPANNAAIVLQLPQGTTLPKGFYAEGSRGG | SARS-CoV-2 | 8 | 0.296 | 0 | 0 | 305 |
| PANNAAIVLQLPQGTTLPKGFYAEGSRGGS | SARS-CoV-2 | 8 | 0.296 | 0 | 0 | 306 |
| ANNAAIVLQLPQGTTLPKGFYAEGSRGGSQ | SARS-CoV-2 | 8 | 0.296 | 0 | 0 | 307 |
| NNAAIVLQLPQGTTLPKGFYAEGSRGGSQA | SARS-CoV-2 | 8 | 0.296 | 0 | 0 | 308 |
| NAAIVLQLPQGTTLPKGFYAEGSRGGSQAS | SARS-CoV-2 | 8 | 0.296 | 0 | 0 | 3 |
| AAIVLQLPQGTTLPKGFYAEGSRGGSQASS | SARS-CoV-2 | 7 | 0.259 | 0 | 0 | 309 |
| AIVLQLPQGTTLPKGFYAEGSRGGSQASSR | SARS-CoV-2 | 8 | 0.296 | 0 | 0 | 310 |
| IVLQLPQGTTLPKGFYAEGSRGGSQASSRS | SARS-CoV-2 | 7 | 0.259 | 0 | 0 | 311 |
| VLQLPQGTTLPKGFYAEGSRGGSQASSRSS | SARS-CoV-2 | 8 | 0.296 | 0 | 0 | 312 |
| LQLPQGTTLPKGFYAEGSRGGSQASSRSSS | SARS-CoV-2 | 8 | 0.296 | 0 | 0 | 313 |
| QLPQGTTLPKGFYAEGSRGGSQASSRSSSR | SARS-CoV-2 | 8 | 0.296 | 0 | 0 | 314 |
| LPQGTFLPKGFYAEGSRGGSQASSRSSSRS | SARS-CoV-2 | 8 | 0.296 | 0 | 0 | 315 |
| PQGTTLPKGFYAEGSRGGSQASSRSSSRSR | SARS-CoV-2 | 6 | 0.222 | 0 | 0 | 316 |
| QGTTLPKGFYAEGSRGGSQASSRSSSRSRN | SARS-CoV-2 | 5 | 0.185 | 0 | 0 | 317 |
| AGNGGDAALALLLLDRLNQLESKMSGKGQQ | SARS-CoV-2 | 3 | 0.111 | 0 | 0 | 318 |
| IGTRNPANNASIVLQLPQGTTLPKGFYAEG | SARS-CoV-2 | 7 | 0.259 | 0 | 0 | 319 |
| GTRNPSNNAAIVLQLPQGTTLPKGFYAEGS | SARS-CoV-2 | 7 | 0.259 | 0 | 0 | PO |
| QLPQGTTLPKGFYAEGSRGGSQASSRSSLR | SARS-CoV-2 | 8 | 0.296 | 0 | 0 | 321 |
| AIVLQLPQMPTLPKGFYAEGSRGGSQAYSR | SARS-CoV-2 | 8 | 0.296 | 0 | 0 | 322 |
| IGTRNPANNAAIVLQLPQGTTLPKGFYAKG | SARS-CoV-2 | 8 | 0.296 | 0 | 0 | 323 |
| GIVNNTVYDPLQPELESFKEELDKYFKNHT | SARS-CoV-2 | 7 | 0.259 | 0 | 0 | 324 |
| VNNTVYDPLQPELESFKEELDKYFKNHTSP | SARS-CoV-2 | 7 | 0.259 | 0 | 0 | 325 |
| YDPLQPELDSFKEELDKYFKNHTSLDVDLG | SARS-CoV-2 | 6 | 0.222 | 0 | 0 | 326 |
| GDAALALLLLDRLNQLESKMSGKGQQQQGQ | SARS-CoV-2 | 3 | 0.111 | 0 | 0 | 4 |
| TGVLTESNKKFLPFQQFGRDIADITDAVRD | SARS-CoV-2 | 1 | 0.037 | 0 | 0 | 327 |
| DFGGFNFSQILPDPSKPSKRSFIEDLLFNK | SARS-CoV-2 | 3 | 0.111 | 0 | 0 | 328 |
| GGFNFSQILPDPSKPSKRSFIEDLLFNKVT | SARS-CoV-2 | 3 | 0.111 | 0 | 0 | 329 |
| NFSQILPDPSKPSKRSFIEDLLFNKVTLAD | SARS-CoV-2 | 5 | 0.185 | 0 | 0 | 330 |
| SQILPDPSKPSKRSFIEDLLFNKVTLADAG | SARS-CoV-2 | 5 | 0.185 | 0 | 0 | 331 |
| ILPDPSKPSKRSFIEDLLFNKVTLADAGFI | SARS-CoV-2 | 4 | 0.148 | 0 | 0 | 332 |
| LPDPSKPSKRSFIEDLLFNKVTLADAGFIK | SARS-CoV-2 | 6 | 0.222 | 0 | 0 | 333 |
| PDPSKPSKRSFIEDLLFNKVTLADAGFIKQ | SARS-CoV-2 | 4 | 0.148 | 0 | 0 | 334 |
| KPSKRSFIEDLLFNKVTLADAGFIKQYGDC | SARS-CoV-2 | 4 | 0.148 | 0 | 0 | 335 |
| PSKRSFIEDLLFNKVTLADAGFIKQYGDCL | SARS-CoV-2 | 3 | 0.111 | 0 | 0 | 336 |
| SKRSFIEDLLFNKVTLADAGFIKQYGDCLG | SARS-CoV-2 | 4 | 0.148 | 0 | 0 | 337 |
| KRSFIEDLLFNKVTLADAGFIKQYGDCLGD | SARS-COV-2 | 3 | 0.111 | 0 | 0 | 338 |
| FIEDLLFNKVTLADAGFIKQYGDCLGDIAA | SARS-CoV-2 | 3 | 0.111 | 0 | 0 | 339 |
| IEDLLFNKVTLADAGFIKQYGDCLGDIAAR | SARS-CoV-2 | 4 | 0.148 | 0 | 0 | 340 |
| EDLLFNKVTLADAGFIKQYGDCLGDIAARD | SARS-CoV-2 | 1 | 0.037 | 0 | 0 | 341 |
| YFKNHTSPDVDLGDIPGINASVVNIQKEID | SARS-CoV-2 | 1 | 0.037 | 0 | 0 | 342 |
| LPDPSKPSKRSFIEDILFNKVTLADAGFIK | SARS-CoV-2 | 7 | 0.259 | 0 | 0 | 343 |
| FSQILPDSSKPSKRSFIEDLLFNKVTLADA | SARS-CoV-2 | 4 | 0.148 | 0 | 0 | 344 |
| FKNLREFVFKNIDGYFNIYSKHTPINLVRD | SARS-CoV-2 | 1 | 0.037 | 0 | 0 | 345 |
| KDFGGFNFSQILPDPSKPSKRSFIEDLLFN | SARS-CoV-2 | 1 | 0.037 | 0 | 0 | 346 |
| GFNFSQILPDPSKPSKRSFIEDLLFNKVTL | SARS-CoV-2 | 2 | 0.074 | 0 | 0 | 347 |
| DVVIGTVNNTVYDPLQPELDSFKEELDKYF | SARS-CoV-2 | 4 | 0.148 | 0 | 0 | 348 |
| VVIGIVNNTVYDPLQPELDSFKEELDKYFK | SARS-CoV-2 | 2 | 0.074 | 0 | 0 | 349 |
| VIGPINNTVYDPLQPELDSFKEELDKYFKN | SARS-CoV-2 | | 0.111 | 0 | 0 | 350 |
| IGIVNNTVYDPLQPELDSFKEELDKYFKNH | SARS-CoV-2 | 4 | 0.148 | 0 | 0 | 351 |
| GIVNNTVYDPLQPELDSFKEELDKYFKNHT | SARS-CoV-2 | 5 | 0.185 | 0 | 0 | 352 |
| IVNNTVYDPLQPELDSFKEELDKYFKNHTS | SARS-CoV-2 | 5 | 0.185 | 0 | 0 | 353 |
| VNNTVYDPLQPELDSFKEELDKYFKNHTSP | SARS-CoV-2 | 6 | 0.222 | 0 | 0 | 354 |
| NNTVYDPLQPELDSFKEELDKYFKNHTSPD | SARS-CoV-2 | 6 | 0.222 | 0 | 0 | 355 |
| VYDPLQPELDSFKEELDKYFKNHTSPDVDL | SARS-CoV-2 | 5 | 0.185 | 0 | 0 | 356 |
| PLQPELDSFKEELDKYFKNHTSPDVDLGDI | SARS-CoV-2 | 6 | 0.222 | 0 | 0 | 357 |
| FKEELDKYFKNHTSPDVDLGDISGINASVV | SARS-CoV-2 | 6 | 0.222 | 0 | 0 | 358 |
| LQLELDSFKEELDKYFKNHTSPDVDLGDIS | SARS-CoV-2 | 5 | 0.185 | 0 | 0 | 359 |
| FKEELDKYFKNHTSLDVDLGDISGINASVV | SARS-CoV-2 | 5 | 0.185 | 0 | 0 | 360 |
| NNTAAIVLQLPQGTTLSKGFYAEGSRGGSA | SARS-CoV-2 | 2 | 0.074 | 0 | 0 | 361 |
| DRLNEVAKNLNESLIDLQELGKYEQYIKWP | SARS-CoV-2 | 1 | 0.037 | 0 | 0 | 367 |
| LNEVAKNLNESLIDLQELGKYEQYIKWPWY | SARS-CoV-2 | 1 | 0.037 | 0 | 0 | 363 |
| KLDDKDPNFKDQVILLNKHIDAYKTFPPTE | SARS-CoV-2 | 1 | 0.037 | 0 | 0 | 364 |
| GAISSVLNDILSRLDKVEAEVQIDRLITGR | SARS-CoV-2 | 1 | 0.037 | 0 | 0 | 365 |
| ISSVLNDILSRLDKVEAEVQIDRLITGRLQ | SARS-CoV-2 | 1 | 0.037 | 0 | 0 | 366 |
| VLNDILSRLDKVEAEVQIDRLITGRLQSLQ | SARS-CoV-2 | 1 | 0.037 | 0 | 0 | 367 |
| LNDILSRLDKVEAEVQIDRLITGRLQSLQT | SARS-CoV-2 | 1 | 0.037 | 0 | 0 | 368 |
| DETQALPQRQKKQQIVTLLPAADLDDFSKQ | SARS-CoV-2 | 3 | 0.111 | 0 | 0 | 369 |
| AVRDPQTLEILDITPCSFGGVSVITPGTNT | SARS-CoV-2 | 1 | 0.037 | 0 | 0 | 370 |
| RATRRIRGGDGKMKDLSPRWYFYYLGTGPE | SARS-CoV-2 | 1 | 0.037 | 0 | 0 | 371 |
| ATRRIRGGDGKMKDLSPRWYFYYLGTGPEA | SARS-CoV-2 | 1 | 0.037 | 0 | 0 | 372 |
| RIRGGDGKMKDLSPRWYFYYLGTGPEAGLP | SARS-CoV-2 | 1 | 0.037 | 0 | 0 | 373 |
| IRGGDGKMKDLSPRWYFYYLGTGPEAGLPY | SARS-CoV-2 | 1 | 0.037 | 0 | 0 | 374 |
| RGGDGKMKDLSPRWYFYYLGTGPEAGLPYG | SARS-CoV-2 | 1 | 0.037 | 0 | 0 | 375 |
| GGDGKMKDLSPRWYFYYLGTGPEAGLPYGA | SARS-CoV-2 | 1 | 0.037 | 0 | 0 | 376 |
| GDGKMKDLSPRWYFYYLGTGPEAGLPYGAN | SARS-CoV-2 | 1 | 0.037 | 0 | 0 | 377 |
| DGKMKDLSPRWYFYYLGTGPEAGLPYGANK | SARS-CoV-2 | 1 | 0.037 | 0 | 0 | 378 |
| GKMKDLSPRWYFYYLGTGPEAGLPYGANKD | SARS-CoV-2 | 2 | 0.074 | 0 | 0 | 379 |
| KMKDLSPRWYFYYLGTGPEAGLPYGANKDG | SARS-CoV-2 | 1 | 0.037 | 0 | 0 | 380 |
| MKDLSPRWYFYYLGTGPEAGLPYGANKDGI | SARS-CoV-2 | 1 | 0.037 | 0 | 0 | 381 |
| GTTLPKGFYAEGSRGGSQASSRSSSRSRNS | SARS-CoV-2 | 1 | 0.037 | 0 | 0 | 382 |
| SPARMAGNGGDAALALLLLDRLNQLESKMS | SARS-CoV-2 | 1 | 0.037 | 0 | 0 | 383 |
| ARMAGNGGDAALALLLLDRLNQLESKMSGK | SARS-CoV-2 | 1 | 0.037 | 0 | 0 | 384 |
| RMAGNGGDAALALLLLDRLNQLESKMSGKG | SARS-CoV-2 | 2 | 0.074 | 0 | 0 | 385 |
| MAGNGGDAALALLLLDRLNQLESKMSGKGQ | SARS-CoV-2 | 2 | 0.074 | 0 | 0 | 386 |
| GNGGDAALALLLLDRLNQLESKMSGKGQQQ | SARS-CoV-2 | 2 | 0.074 | 0 | 0 | 387 |
| NGGDAALALLLLDRLNQLESKMSGKGQQQQ | SARS-CoV-2 | 1 | 0.037 | 0 | 0 | 388 |
| GGDAALALLLLDRLNQLESKMSGKGQQQQG | SARS-CoV-2 | 2 | 0.074 | 0 | 0 | 389 |
| DAALALLLLDRLNQLESKMSGKGQQQQGQT | SARS-CoV-2 | 1 | 0.037 | 0 | 0 | 390 |
| LALLLLDRLNQLESKMSGKGQQQQGQTVTK | SARS-CoV-2 | 1 | 0.037 | 0 | 0 | 391 |
| LLDRLNQLESKMSGKGQQQQGQTVTKKSAA | SARS-CoV-2 | 1 | 0.037 | 0 | 0 | 392 |
| RATRRIRGGDGKMKYLSPRWYFYYLGTGPE | SARS-CoV-2 | 1 | 0.037 | 0 | 0 | 393 |
| NAAIVLQLPQGTTLSKGFYAEGSRGGSQAS | SARS-CoV-2 | 1 | 0.037 | 0 | 0 | 394 |
| SDAALALLLLDRLNQLESKMSGKGQQQQSQ | SARS-CoV-2 | 1 | 0.037 | 0 | 0 | 395 |
| GKMKDLSPRWYFYYLGTGPEAGLLYGANKD | SARS-CoV-2 | 1 | 0.037 | 0 | 0 | 396 |
| GTTLPKGFYAEGSRGGSQASSRYSSRSRNS | SARS-CoV-2 | 1 | 0.037 | 0 | 0 | 397 |
| DGKMKDLSPRWYFYYLGTGSEAGLPYGANK | SARS-CoV-2 | I | 0.037 | 0 | 0 | 398 |
| NGGDAALALLLLDRLNQLETKMSGKGQQQQ | SARS-CoV-2 | 1 | 0.037 | 0 | 0 | 399 |
| VKQIYKIPPIKDFGGFNFSQILPDPSKPSK | SARS-CoV-2 | 1 | 0.037 | 0 | 0 | 400 |
| QMAYRFNGIGVTQNVLYENQKLIANQFNSA | SARS-CoV-2 | 1 | 0.037 | 0 | 0 | 401 |
| LYQDVNCTEVPVAIHADQLTPTWRVYSTGS | SARS-CoV-2 | 1 | 0.037 | 0 | 0 | 402 |
| YQDVNCTEVPVAIHADQLTPTWRVYSTGSN | SARS-CoV-2 | 1 | 0.037 | 0 | 0 | 403 |
| TEVPVAIHADQLTPTWRVYSTGSNVFQTRA | SARS-CoV-2 | I | 0.037 | 0 | 0 | 404 |
| EVPVAIHADQLTPTWRVYSTGSNVFQTRAG | SARS-CoV-2 | 1 | 0.037 | 0 | 0 | 405 |
| FNFSQILPDPSKPSKRSFIEDLLFNKVTLA | SARS-CoV-2 | 1 | 0.037 | 0 | 0 | 406 |
| RSFIEDLLFNKVTLADAGFIKQYGDCLGDI | SARS-CoV-2 | 1 | 0.037 | 0 | 0 | 407 |
| SFIEDLLFNKVTLADAGFIKQYGDCLGDIA | SARS-CoV-2 | 1 | 0.037 | 0 | 0 | 408 |
| GRRGPEQTQGNFGDQELIRQGTDYKHWPQI | SARS-CoV-2 | 1 | 0.037 | 0 | 0 | 409 |
| SFIEDLLFNKVTLADVGFIKQYGDCLGDIA | SARS-CoV-2 | 1 | 0.037 | 0 | 0 | 410 |
| PNITNLCPFGEVFNATRFTSVYAWNRKRIS | SARS-CoV-2 | 1 | 0.037 | 0 | 0 | 411 |
| SFIEDLLFNKVTLADAGFIKQYGDCLGDIV | SARS-CoV-2 | 1 | 0.037 | 0 | 0 | 412 |
| LQKTVQIKNPKKQAPESFLHTWDFRRGFVT | Torque teno midi virus | 1 | 0.037 | 0 | 0 | 413 |
| QQHTKLQLLQLINNLKKKQKLIQLQTGILD | Torque teno midi virus 11 | 2 | 0.074 | 1 | 0.048 | 414 |
| QIENPERQDPRSILHQWDYRRGFIKERALK | Torque teno midi virus 12 | 1 | 0.037 | 0 | 0 | 415 |
| IQIVNPEKQSPETIIHPWDYRRGLIKEKAL | Torque teno midi virus 7 | 4 | 0.148 | 0 | 0 | 416 |
| KAMLRDWDYRRGIITTTALKRMSEHLQTDS | Torque teno midi virus 8 | 1 | 0.037 | 0 | 0 | 417 |
| ENLQQLILHQQQQQQKLKSNILKLLMDLKH | Torque teno midi virus 8 | 2 | 0.074 | 1 | 0.048 | 418 |
| SQEIPQTQNLQELIQQQQQQQQQLKYNILK | Torque teno midi virus 9 | 2 | 0.074 | 1 | 0.048 | 419 |
| QLQQQLQFLTREMFKTQAGLHINPMLLNQR | Torque teno virus | 2 | 0.074 | 1 | 0.048 | 420 |

There was no overlap between the reactive peptides observed in the convalescent and negative control samples (FIG. 2C). These enriched peptides clustered together into nine reactive regions of the S protein and six reactive regions of the N protein (FIG. 2C), which represents a minimum estimate for the number of epitopes. These epitopes were recognized at a range of prevalences across the sampled population. The most widely-recognized epitopes in S (positions 795-848 and 1127-1177) and N (positions 140-193) were each detected in 41-68% of the convalescent samples that tested positive with our assay (n=22) (FIG. 2C), and >95% (21/22) of these convalescent samples were reactive to at least one of these three immunodominant regions. At the other extreme, six (43%) of the observed epitope regions were each detected in only a single donor. Despite the detection of a variety of SARS-CoV-2 S epitopes in the convalescent donors, very little reactivity was detected to peptides within the RBD, suggesting that these epitopes require protein conformations that are not well represented by linear 30mers.

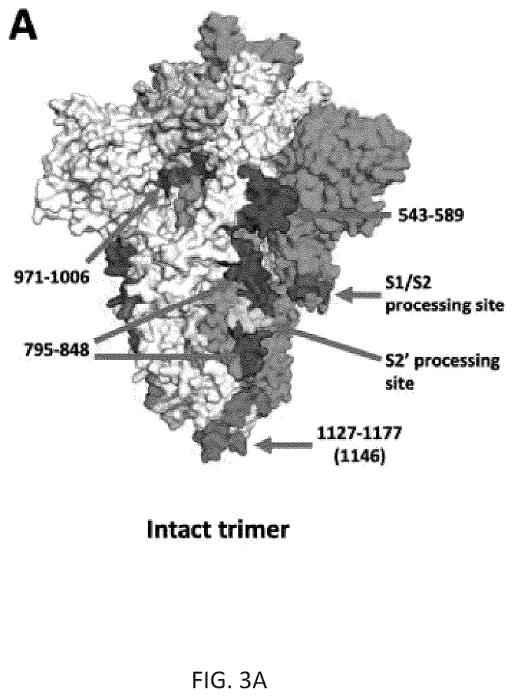

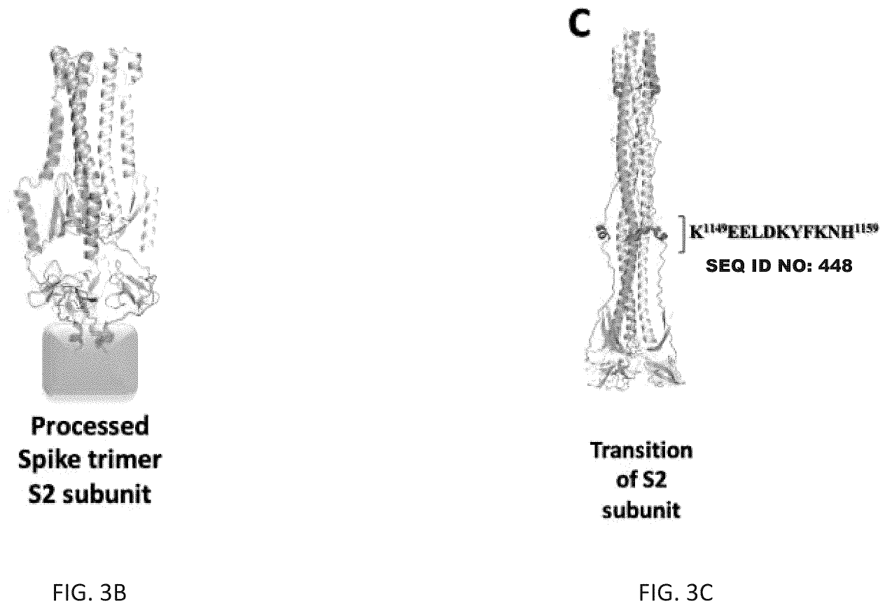

To evaluate the potential for the identified S protein epitopes to be targeted by neutralizing antibodies, we evaluated these within the context of the protein's structure. Of the S epitopes identified, four were recurrent across multiple convalescent samples, occurring at positions 1127-1177, 795-848, 543-589 and 971-1006 (of SARS-Cov-2 S protein sequence GenBank: YP_009724390.1) and found in 14/27, 11/27, 4/27 and 2/27 convalescent donors, respectively. The enriched peptides at each of these four high-confidence regions were mapped onto a rendering of the recently-solved 3-dimensional structure of the native S trimer (FIG. 3A). FIG. 3A shows a space-filling model showing the native SARS-CoV-2 Spike trimer (monomers shown in green, gray and white) with the 4 recurrent epitope regions targeted by COVID-19 convalescent IgG (see also FIG. 2C) highlighted in blue or magenta. Each epitope is identified by its amino acid range within the S protein sequence (GenBank: YP_009724390.1). Protease processing sites are also highlighted, including the S2′ site that occurs within the 795-848 epitope.

All four epitope regions are accessible for antibody binding on the surface of the trimer. The most widely-recognized reactive region (1127-1177) is located within the ‘stem helix’ just upstream and partially overlapping with the heptad repeat region 2 (HR2); this region is proximal to the transmembrane domain and partially unresolved in the native structure. The second epitope (795-848) resides at the S2′ cleavage site, spanning the fusion peptide whose exposure and incorporation into the host membrane are essential steps in virus entry into cells. Comparison of pre-and post-fusion structures (FIGS. 3B and 3C) indicates that the HR2 epitope lies within a region that undergoes a dramatic conformational rearrangement during fusion. FIG. 3B shows a ribbon model of the S2 subunit after protease processing. The epitope at positions 1127-1177 (magenta) includes a region unresolved in the structure (marked by blue box at the bottom of the structure). FIG. 3C shows a ribbon model of the 6-helical bundle (post-fusion) conformation of the S2 subunit. The 1127-1177 region is again highlighted in magenta, and a comparison with FIG. 3B shows the dramatic conformational rearrangement that occurs at this site.

### Antibody Epitopes and Protein Conservation Across the Human CoVs

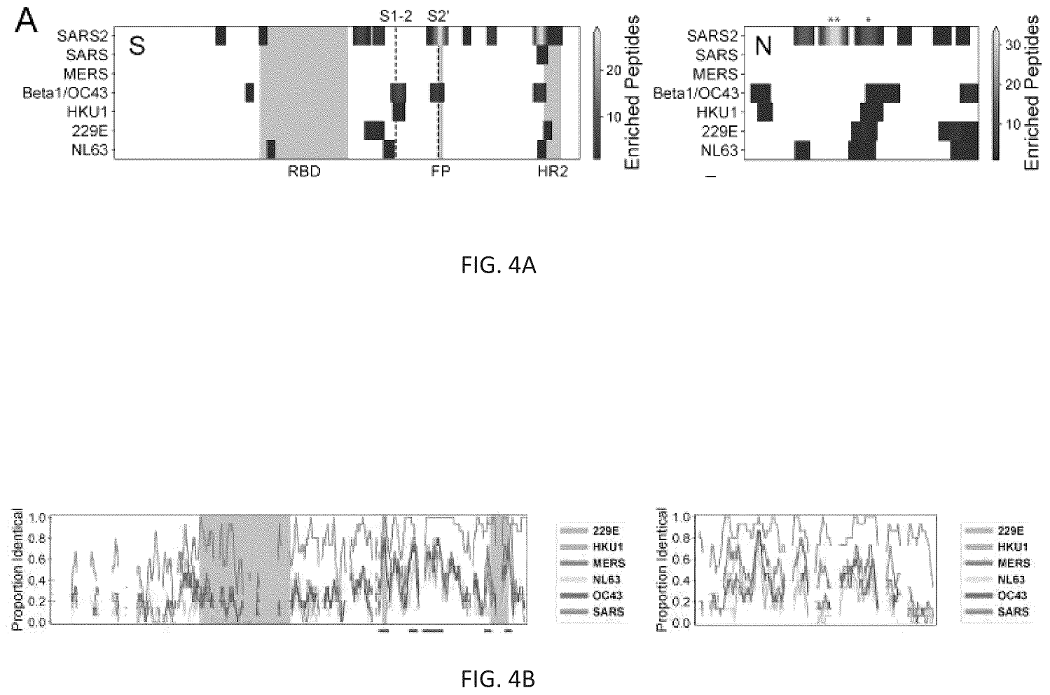

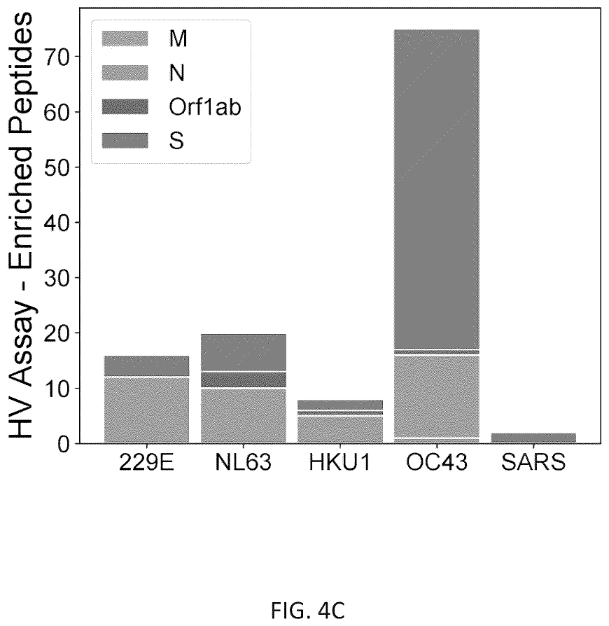

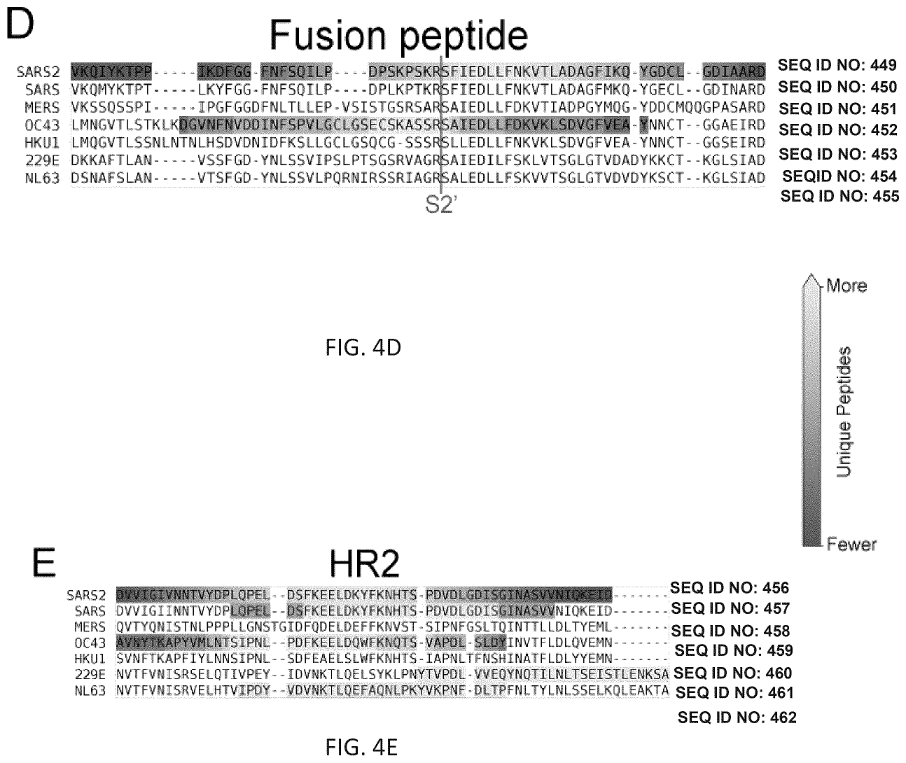

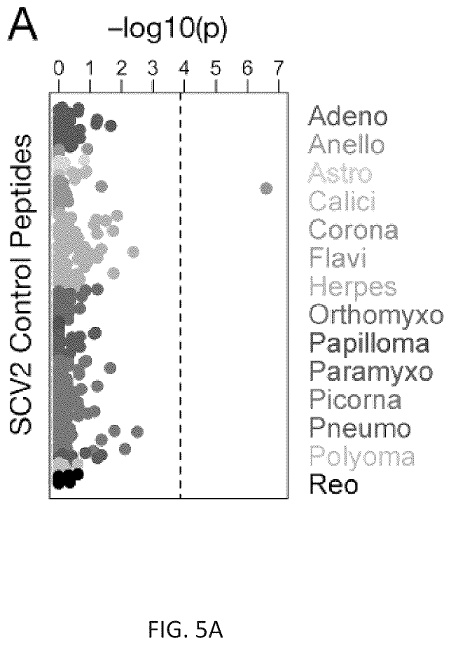

To compare the SARS-CoV-2 reactivity profile described above with those of the other human coronaviruses, we performed a similar analysis but using the HV library (which covers all of the endemic human CoVs) and focusing on pre-pandemic donors. Applying the same Z-score threshold described above to the HV library, we identified reactivity to at least one endemic human coronavirus in 17 (51.5%) of the negative control samples we tested (n=33). To avoid false positives, we required ≥2 enriched peptides for a sample to be considered seropositive. Across all of the different coronaviruses, the vast majority of the recognized peptides were from the S and N proteins (95% of all enriched coronavirus peptides), with occasional reactivity observed to peptides in Orflab and a single peptide from the Membrane (M) protein recognized in one sample (FIG. 4C). FIG. 4C shows protein-level distribution of enriched HV library peptides across five HCoVs and 33 pre-pandemic control samples. A single peptide could be counted multiple times if enrichment was independently observed in multiple samples. Along with our SCV2 library data, these results indicate that S and N are the predominant antibody targets across all of the human-infecting coronaviruses and that pre-existing anti-CoV reactivity is common in the pre-pandemic population.

Within the S protein, we observed reactivity to homologous regions across multiple coronavirus species with highly variable percent identity to SARS-CoV-2 depending on the region and virus species (12.1-92.5% identical, average=40%) (FIG. 4A). FIG. 4A shows heat maps illustrating the relative locations of enriched SCV2 (from COVID-19 convalescent samples) and HV (from pre-pandemic controls) library peptides within the S (left) and N (right) proteins and across all human-infecting coronaviruses. Results have been aggregated across all tested samples and the color at each location indicates the number of unique enriched peptides. The vertical dashed lines in the S protein plot represent the S1-S2 and S2′ cleavage sites, respectively. Above the N plot, ‘**’ and ‘*’ indicate the 1st and 2nd most commonly immunogenic regions of this protein in COVID-19 convalescent samples, respectively.

Notably, we observed a correlation between amino acid sequence conservation among members of the Betacoronavirus genus and peptide enrichment in our assay. Across the full S protein, we identified five highly conserved regions (≥70% identical across 15mer sliding windows, blue bars in FIG. 4B) between SARS-CoV-2 and each of the two endemic human betacoronaviruses: hCoV-OC43 and hCoV-HKU1 (four shared, one unique to each virus). All of these regions were located within the S2 subunit (FIG. 4B), and while enriched SARS-CoV-2 peptides covered only 37% of the full S protein, we observed enriched peptides across almost all of the highly conserved regions: 96.4% (107/111) and 75.6% (93/123) of residues within these highly-conserved regions overlapped ≥1 enriched peptide for hCoV-OC43 and hCoV-HKU1, respectively. FIG. 4B shows a comparison of amino acid sequence identity between SARS-CoV-2 and the other six human CoVs across the same S and N alignments used in FIG. 4A. A sliding window of 15 amino acids was used and gaps represent windows with ≥30% indels. Blue bars under the S plot indicate regions ≥15 amino acids long that exhibit ≥70% identity between SARS-CoV-2 and hCoV-OC43 and/or hCoV-HKU1. Grey boxes in FIGS. 4A and 4B indicate selected functional domains: receptor binding domain (RBD), fusion peptide (FP) and heptad repeat 2 (HR2).

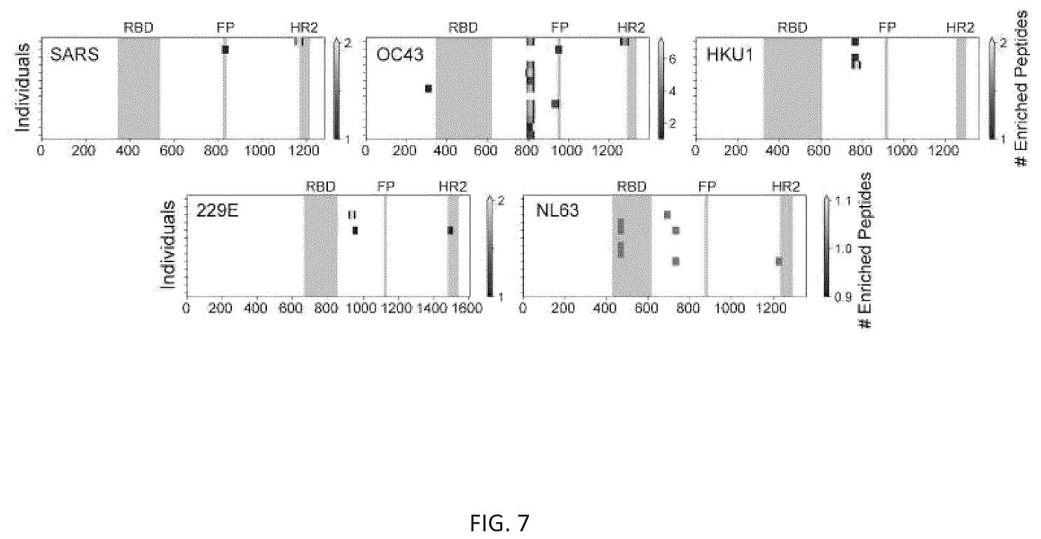

Across the different coronavirus species, the most commonly recognized S protein region, HR2, is also the most commonly reactive SARS-CoV-2 region within our convalescent sera. We detected reactivity in this region to ¾ of the endemic human coronaviruses, though the precise locations of the recognized epitopes likely vary somewhat between species (FIG. 4E). In one pre-pandemic serum sample, we also observed two enriched peptides in this region from the closely-related, epidemic-associated SARS-CoV species; however, these enrichments likely result from cross-reactivity with the endemic hCoV-OC43 (FIG. 7). For the Betacoronavirus 1 species (beta-CoV-1), which includes hCoV-OC43, we also detected reactivity at the same position as the second most immunodominant SARS-CoV-2 epitope, which overlaps the fusion peptide and S2′ cleavage site (FIG. 4A). At this epitope region, we observed enrichment of one SARS-CoV peptide within a sample that also exhibited reactivity to homologous hCoV-OC43 peptides, again consistent with cross-reactive antibodies. However, the minimal epitope region contained within all enriched peptides is distinct between beta-CoV-1/hCoV-OC43 and SARS-CoV-2 (yellow residues in FIG. 4D).

FIGS. 4C and 4D show multiple sequence alignments of the immunodominant and most widely-recognized protein regions of SARS-CoV-2, including representative sequences from each of the seven human coronaviruses. Regions containing enriched peptides are highlighted by colored backgrounds, with bright yellow indicating residues contained within the most unique enriched peptides and dark green indicating those contained within the least unique enriched peptides. SARS-CoV-2 reactivity was determined using the SCV2 peptide library, while reactivity for the other coronaviruses was determined using the HV peptide library.

In contrast, we did not observe any reactivity for the endemic coronaviruses within the most commonly immunogenic SARS-CoV-2 N protein region (shown by double asterisks in FIG. 4A). However, we did observe homologous reactivities in other portions of the N protein. In fact, the second most commonly immunogenic region observed in our COVID-19 convalescent samples (positions 206-252 in FIG. 2C) overlaps with immunogenic regions in all four endemic human coronaviruses (shown by a single asterisk “*” in FIG. 4A). Somewhat surprisingly, however, we observed a somewhat greater similarity in the locations of reactive epitopes between SARS-CoV-2 and the endemic alphacoronaviruses (hCoV-229E and hCoV-NL63) than we did with the endemic betacoronaviruses (hCoV-HKU1 and hCoV-OC43) (FIG. 4A).

### Recurrent Interspecies Cross-Reactivity Elicited by SARS-CoV-2 Exposure

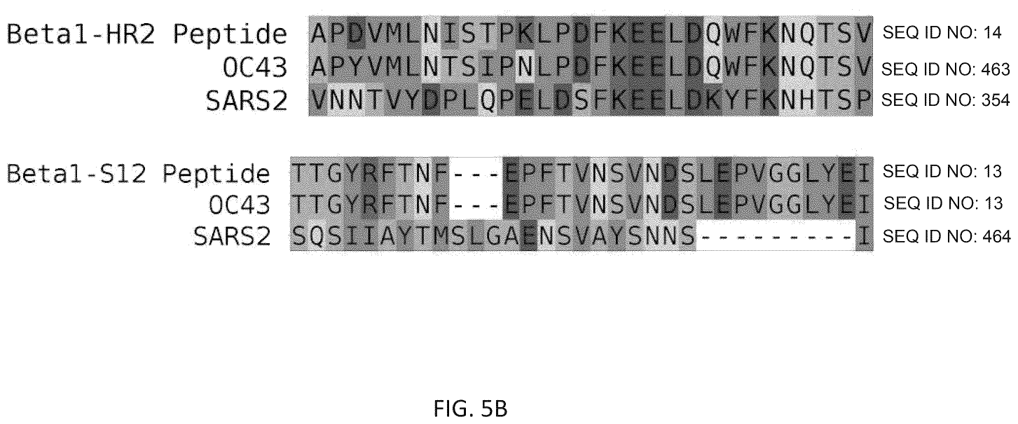

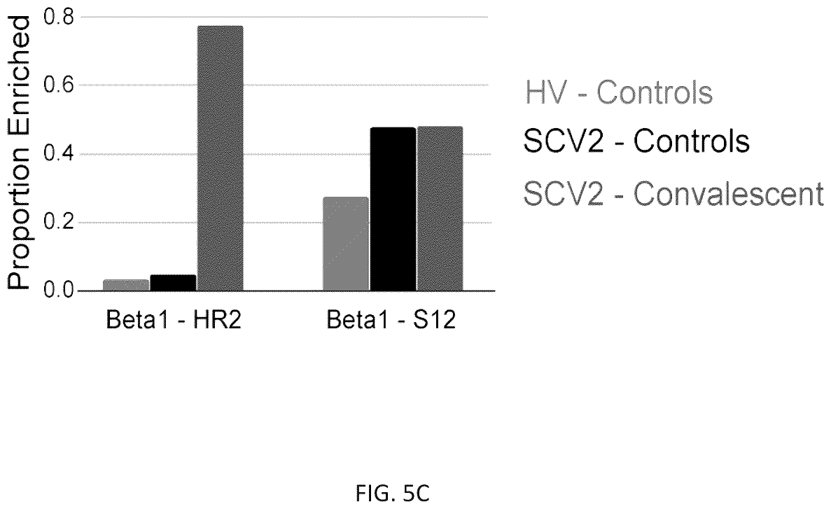

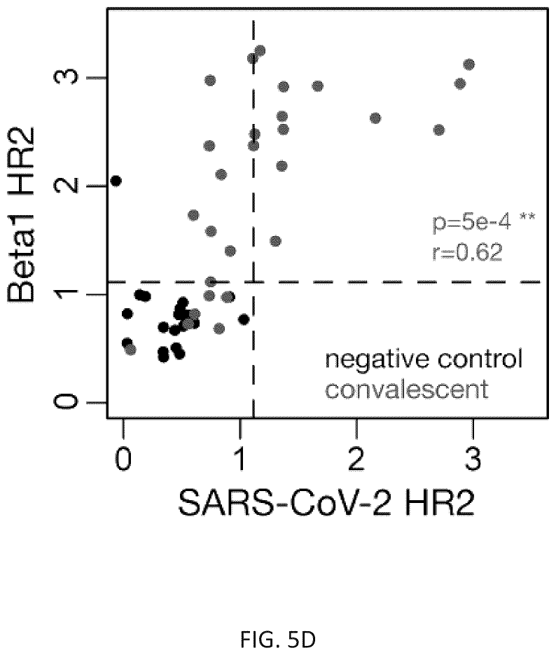

To explore the possibility that the antibody response to SARS-CoV-2 cross-reacts with other viruses, we focused on the panel of control peptides present in both the SCV2 and HV libraries (FIG. 5A). FIG. 5A shows a Fisher's exact test p-values measuring the correlation between donor SARS-CoV-2 status and reactivity for each of 393 control peptides. These peptides were designed from 55 virus species that belong to 14 different families (colors, labels correspond to family names with the omission of “-viridae”), and they recognize epitopes that we previously identified as commonly reactive in the general population. The dashed vertical line shows the Bonferroni-corrected threshold for significance.

This panel comprises 393 peptides from 55 virus species (range: 1-11 per species; 22 from the Coronaviridae family) for which we have previously observed recurrent reactivity in the general population. Consistent with previous results (not shown) and expected viral prevalences, we observed a range of positivity rates, including 25-100% for Rhinovirus-derived peptides and 0-48% for endemic human CoV-derived peptides. Comparing convalescent and negative control groups, Fisher's exact tests identified a single peptide as significantly different between the groups at a Bonferroni-corrected threshold of p<1.3e−4 (FIG. 5A). This peptide was enriched in 21/27 convalescents and 1/21 controls (p=2.5e−7), and was designed from a beta-CoV-1 strain. Although it was designed from a bovine coronavirus sequence, the peptide is 86.7% identical to the corresponding 30mer region in hCoV-OC43 (26/30 identical residues) and 100% identical to hCoV-OC43 across the 18 C-terminal residues. This peptide (SEQ ID NO: 14) corresponds to positions 1218-1247 of the beta-CoV-1 Spike protein and it precisely overlaps the immunodominant HR2 region we identified based on SARS-CoV-2 peptides. Therefore, we hereafter refer to this peptide (SEQ ID NO: 14) as ‘Beta1-HR2’. Beta1-HR2 also exhibits a high degree of conservation with SARS-CoV-2, particularly in the C-terminal portion of the peptide (66.7% identical across 18 C-terminal residues) (FIG. 5B). FIG. 5B shows sequence alignments between SARS-CoV-2 (SARS2) and the Betacoronavirus 1 strain, human coronavirus OC43 (OC43), at two Spike protein regions covered by SCV2 library control peptides designed from Betacoronavirus 1 (Beta1) sequences. Residues are colored according to amino acid properties: small non-polar (orange), hydrophobic (green), polar (pink), negatively charged (red) and positively charged (blue).

By comparison, a second Spike-derived beta-CoV-1 peptide (‘Beta1-S12’) was reactive in about half of all samples tested (13/27 convalescents and 10/21 controls), indicating a high level of exposure to beta-CoV-1/hCoV-OC43 that did not differ between the groups (FIG. 5C). FIG. 5C shows the proportion of samples reactive to the two Betacoronavirus 1 peptides shown in FIG. 5B. Two separate sets of negative controls are shown, those assayed with the HV peptide library (grey, n=33) and those assayed with the SCV2 peptide library (black, n=21). In FIG. 5C, results from COVID-19 convalescent samples are shown in red (n=27). Notably, the sequences of SARS-CoV-2 and beta-CoV-1 are highly divergent at the region covered by Beta1-S12 (13.3% identical) (FIG. 5B).

To further test the hypothesis that reactivity to Beta1-HR2 represents cross-reactivity with SARS-CoV-2, we compared donor-level reactivity to this peptide and the homologous SARS-CoV-2 peptide, referred to herein as SARS-CoV-2-HR2 (FIG. 5D). FIG. 5D shows a quantitative comparison of reactivities to homologous HR2 peptides from SARS-CoV-2 and Beta1-CoV across the donor cohort. Axes represent log10 (2+Z-scores) and dashed lines indicate threshold for significance (Z-score ≥11). We observed a significant positive correlation between measured reactivity against these two peptides in convalescent donors (r=0.62, p=5e−4), and all of the donors reactive to SARS-CoV-2-HR2 were also reactive to Beta1-HR2. However, an additional six convalescent donors were reactive to Beta1-CoV, despite a lack of reactivity to any SARS-CoV-2 peptides overlapping the HR2 epitope. Moreover, for donors reactive to either HR region, the signal strength for Beta1-HR2 was up to ˜170-fold (mean ˜10-fold) higher than for SARS-CoV-2-HR2, indicating that the anti-HR2 antibodies elicited by SARS-CoV-2 infection actually bind better to Beta1-HR2.

### Discussion

Like most viruses, SARS-CoV-2 elicits a robust antibody response whose targets are likely to be important determinants of disease outcome and the extent of protection conferred following natural infection or vaccination. In this study, we describe a customizable platform that enables epitope-resolved profiling of the antibody response (‘PepSeq’), and its application to the study of human CoVs including SARS-CoV-2. Using this system, we identify immunodominant epitopes in both the S and N proteins, several of which overlap conserved, functional sites in the Spike S2 subunit, and therefore have the potential to be sites of broadly neutralizing reactivity. By examining reactivity in pre-pandemic donors to homologous peptides from multiple human CoVs, we also show that the response to SARS-CoV-2 strongly cross-reacts with an endemic human CoV at one of these epitopes.

By independently testing reactivity across thousands of potential epitopes, we identified several with promise for use in both diagnostics and functional characterization assays. For two of the epitopes, we detected in the S2 subunit of Spike structural considerations, as well as previous characterization of related epitopes (Keng et al., 2005; Lai et al., 2005; Poh et al., 2020), strongly indicate neutralization potential. In these cases, a peptide-based assay may provide a facile means of profiling functional reactivities independently of cell/viral culture, and in a way that complements ACE2: RBD binding inhibition assays that cannot measure S2 reactivity (Tan et al.). We also identified a set of 4 peptides across the S and N proteins that together exhibit great potential for generating an accurate profile of SARS-CoV-2 exposure. Although the precise diagnostic performance of this particular set needs to be quantified on a larger, independent sample set, our results provide a blueprint for a new generation of peptide-based diagnostics that would be easier to manufacture, and in some cases more informative, than existing full-protein/domain assays.

Our PepSeq analysis identified a novel epitope contained within positions 1127-1177 in Spike, where the minimal reactive sequence is FKEELDKYF (SEQ ID NO: 421), as the most widely-recognized SARS-CoV-2 linear epitope target in convalescent donors (FIG. 2C). This region is located within the ‘stem helix’, directly N-terminal of the heptad-repeat 2 (HR2) region. While largely unresolved in the prefusion structure, analysis of post-fusion structures of CoV Spike proteins indicate that HR2 undergoes a ˜180° reorientation during the formation of the 6-helix bundle in which it comes into close contact with the heptad-repeat 1 (HR1) region (Walls et al., 2017). HR-derived peptides that disrupt the HR1: HR2 interaction have previously been shown to inhibit infection by other CoVs (Xia et al., 2019) (Liu et al., 2004), highlighting the strong potential for functional targeting of this region. Moreover, neutralizing monoclonal antibodies raised against related CoVs, including SARS-CoV (which has >95% amino acid-level identity at the stem helix of HR2), have been shown to bind a region directly adjacent to the one that we identified in this study (Routledge et al., 1991) (Lai et al., 2005) (Keng et al., 2005). Strikingly, our analysis of reactivity across the human-infecting CoVs indicated that sites in the proximity of HR2 are also recognized in the responses to at least three of the four endemic species (FIG. 4A). Since portions of this region are highly conserved across species (FIG. 4B), cross-reactivity with pre-existing anti-CoV antibodies likely accounts for some of its immunodominance in the response to SARS-CoV-2.

A second immunodominant reactivity that we identified in Spike S2 also occurs in a region whose sequence is highly-conserved across CoV species: positions 795-848, where the minimal reactive sequence is EDLLEN (SEQ ID NO: 422), which overlaps the S2′ cleavage site and the Fusion Peptide (FP). Since the minimal region needed to explain the reactive peptides included residues on both sides of S2′ in many donors, this reactivity has the potential to block proteolytic processing and thereby prevent maturation of the S protein. Alternatively, and perhaps additionally, binding of antibody to the FP is expected to prevent its insertion into the host membrane and therefore prevent fusion and cell entry. A recent study, using a lower-throughput peptide-based approach also identified this FP epitope as reactive in two SARS-CoV-2 convalescent donors, and while they did not characterize the mechanism of action, they demonstrated the neutralization potential of antibodies against this epitope using antibody depletion assays (Poh et al., 2020). This study also reported an epitope downstream of the Spike RBD to which antibodies also exhibited neutralization potential. We observed reactivity to this same epitope in four of our SARS-CoV-2 convalescent donors, the epitope have positions 543-589, where the minimal reactive sequence is LPFQQFGRDIADT (SEQ ID NO: 423)). In addition to Spike S2 epitopes, we detected an immunodominant reactivity at positions 140-193 (see also SEQ ID NO: 3) of the SARS-CoV-2 nucleocapsid (N) protein, which lies at the C-terminal end of the domain that is primarily responsible for binding viral RNA (Chang et al., 2009). Unlike the reactivities described in Spike S2, this region does not appear to be targeted in the response to other CoVs (FIG. 4A).

Despite well-documented serological reactivity in studies using the full-length RBD antigen (Amanat et al., 2020), we observed very little reactivity to peptides designed from the RBDs of human CoVs, including SARS-CoV-2 (FIGS. 2C and 3A). This lack of reactivity in our assay, as well as a similar absence of reactivity in a recent study using a lower-throughput peptide-based approach (Poh et al., 2020), suggests that antibodies to the RBD recognize conformational epitopes and/or depend on post-translational modifications. Like other peptide-based antibody assays, PepSeq is limited to the detection of epitopes that are well-represented by short linear peptides and do not require post-translational modifications. The dependence of RBD epitopes on secondary/tertiary structure is supported by structural analyses of the footprints of neutralizing antibodies bound to Spike RBD, which indicate the involvement of residues that are distal in the linear sequence (Pinto et al., 2020; Yuan et al.). The identification of epitopes like these will require lower throughput approaches including mutagenesis and/or structural studies.

The observation that ˜80% of SARS-CoV-2 convalescent donors react strongly to a Beta1-HR2 peptide targeted in ˜5% of our negative control samples (FIG. 5C) is, to our knowledge, the first identification of a B cell epitope for which there is cross-reactivity between the pandemic virus and an endemic pathogen. The fact that antibodies against Beta1-HR2 occur in individuals who also have antibodies targeting SARS-CoV-2-HR2, but with, on average, approximately 5× greater signal strength, is most consistent with a model in which pre-existing B cell clones raised against hCoV-OC43 are recruited into the response to SARS-CoV-2. In further support of this hypothesis, the one pre-pandemic donor in which we observed a strong Beta1-HR2 response with our HV assay also exhibited reactivity to two HR2 peptides designed from SARS-CoV (no SARS-CoV-2 peptides are present in our HV library) (FIG. 7). Pre-existing cross-reactive clones would be expected to have a range of intrinsic affinities for the homologous SARS-CoV-2 epitope, and these could be further improved by somatic mutation. However, by analogy with other viruses, the fact that presumed exposure to OC43 precedes exposure to SARS-CoV-2 may limit the efficiency with which the response can be redirected, due to ‘imprinting’ (Gostic et al., 2016; Monto et al., 2017), which could account for the systematic difference in affinities to the corresponding epitopes from the two species. Under this model, the ˜20% of convalescent donors who exhibit detectable reactivity to Beta1-HR2 but not to SARS-CoV-2-HR2 (upper left quadrant of FIG. 5D) represent cases where pre-existing antibodies to OC43 bind only weakly to SARS-CoV-2 (below the threshold of the PepSeq assay) and have been unable to acquire a high affinity against the new virus. This model also suggests that anti-Beta1-HR2 B cell memory that is capable of cross-reacting with SARS-CoV-2 is prevalent in the general population-consistent with the near universal seropositivity reported for HCoV-OC43 (Gorse et al., 2010)-although often below our limit of detection. Our findings raise the possibility that the nature of an individual's antibody response to prior hCoV-OC43 infection may impact the course of COVID-19 disease.

The HR2 cross-reactivity characterized here represents a possible source of background reactivity for SARS-CoV-2 serological assays that include the S2 subunit of Spike, which would be absent in those targeting only the RBD, for which sequence conservation is lower across species (Khan et al., 2020). Furthermore, based on the level of sequence conservation at the S2′ cleavage/fusion peptide site, we expect that similar cross-reactivity may also occur at this site, and, in fact, we observed preliminary evidence for such cross-reactivity in one of the pre-pandemic controls analyzed with our HV library (FIG. 7). In FIG. 7, each row represents a pre-pandemic negative control sample that was determined to be seropositive for at least one of the non-SARS-CoV-2 human infecting coronaviruses (i.e., enrichment of ≥2 peptides from a non-SARS-CoV-2 coronavirus). The same 13 samples are shown in the same order in each plot. The focal coronavirus species is indicated in the top left corner of each plot: SARS-CoV (‘SARS’), Beta1-CoV/hCoV-OC43 (‘OC43’), hCoV-HKU1 (‘HKU1’), hCoV-229E (‘229E’), and hCoV-NL63 (‘NL63’). Each position is colored according to the number of enriched peptides that overlap that position. Grey boxes indicate selected functional regions: receptor binding domain (RBD), fusion peptide (FP) and heptad repeat 2 (HR2). Both samples exhibiting reactivity to SARS-CoV peptides (top two rows) also exhibit hCoV-OC43 reactivity in homologous regions, consistent with cross-reactivity between peptides derived from endemic and epidemic coronavirus species. Both serum samples exhibiting reactivity to SARS-CoV peptides were collected in 2019 (16 years after the SARS-CoV epidemic) in Bethesda, MD, USA. Given the timing of these samples and the very small number of documented SARS-CoV cases in the US (Centers for Disease Control and Prevention (CDC), 2003), it is highly unlikely that these individuals have actually been exposed to SARS-CoV.

Due to the absence of an hCoV-OC43 S2′ control peptide in our SCV2 library, we were not able to directly evaluate the potential for this cross-reactivity in COVID-19 convalescent donors. Nonetheless, our findings indicate that analysis of S2 reactivity is crucial for a complete assessment of the humoral response to SARS-CoV-2 and is consistent with the observation that S2-only assays provide an equally strong correlate of neutralization compared to RBD-only assays. Our findings also indicate that the incorporation of related beta-CoV antigens may improve the sensitivity of SARS-CoV-2 serological analyses, and in particular, that a differential analysis of SARS-CoV-2 and hCoV-OC43 Spike reactivity may provide an important measure of the efficiency with which pre-existing cross-reactive responses can be redirected.

The identification of broadly-immunogenic epitopes in conserved functional domains of SARS-CoV-2 Spike S2, including cross-reactivity with an endemic human CoV, also has implications for the design of therapeutic antibodies and vaccines. SARS-CoV-2 vaccines currently under development predominantly use 2 forms of the S antigen—whole protein or the RBD—and in each case are designed primarily to elicit neutralizing antibodies. Relative to RBD-focused vaccines, we hypothesize that vaccines that include the Spike HR2 and FP sites: (i) will be able to induce a broader array of neutralizing reactivities, (ii) may be more capable of rapidly recruiting pre-existing memory B cells that are prevalent in the population and (iii) may be less prone to viral escape due to a lower tolerance for amino acid substitutions. In particular, the identification of HR2 as a conserved, functionally-important and broadly-immunogenic site capable of eliciting cross-reacting antibodies, makes this region a candidate for the development of broadly-neutralizing responses against betacoronaviruses.

The epitope regions identified herein may be used in peptide-based diagnostics, development of subunit vaccines, and development of therapeutic binders, including monoclonal antibodies. The correlation between reactivity to SARS-CoV-2 and the endemic Coronavirus may be used to develop enhanced (more sensitive, more informative) diagnostic assays.

### Characterization of Additional SARS-CoV-2 Antibody Epitopes

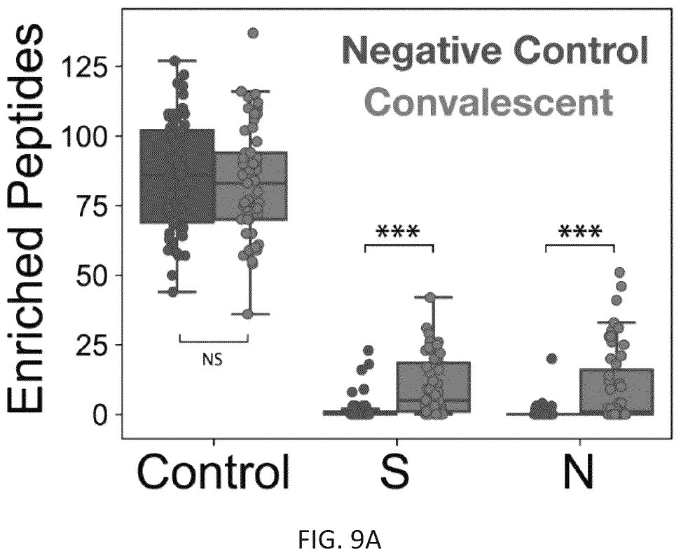

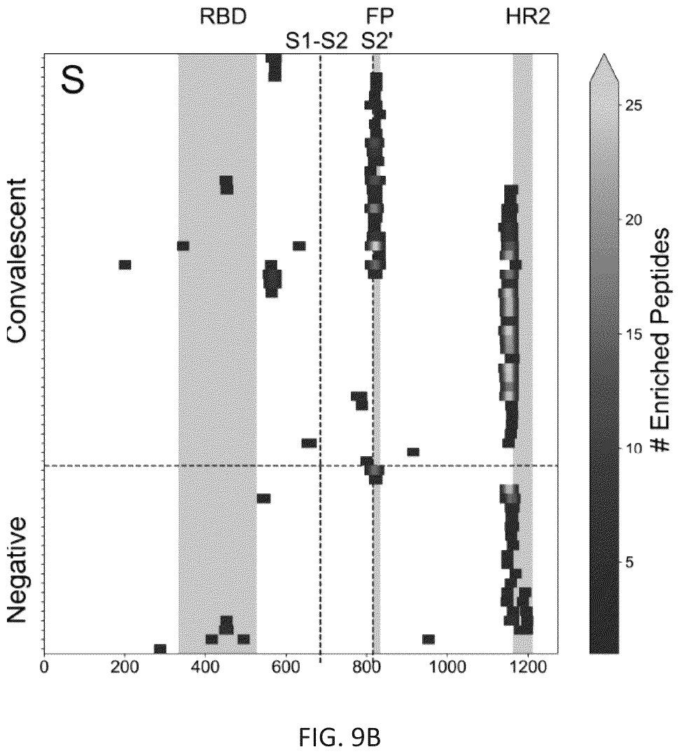

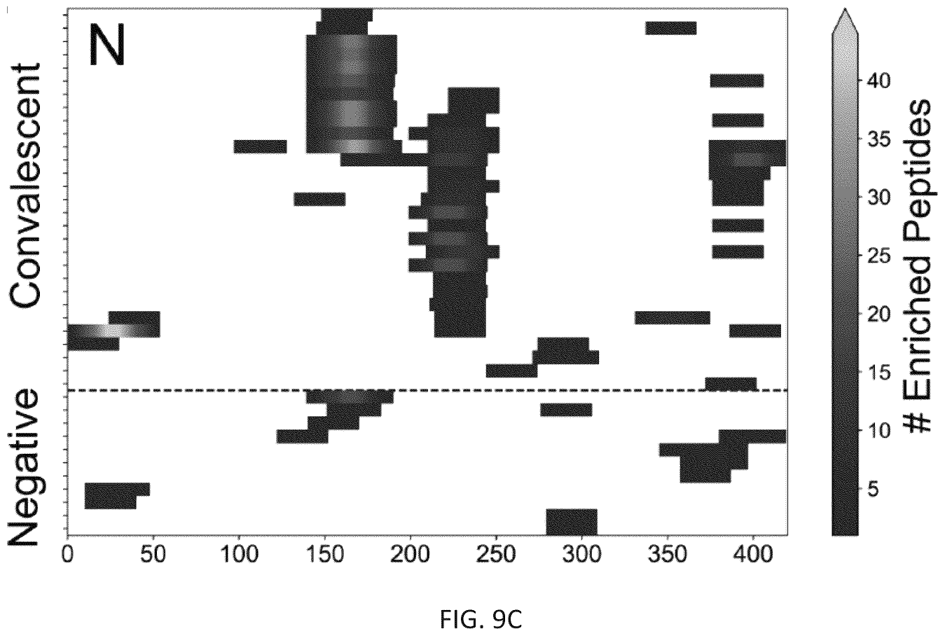

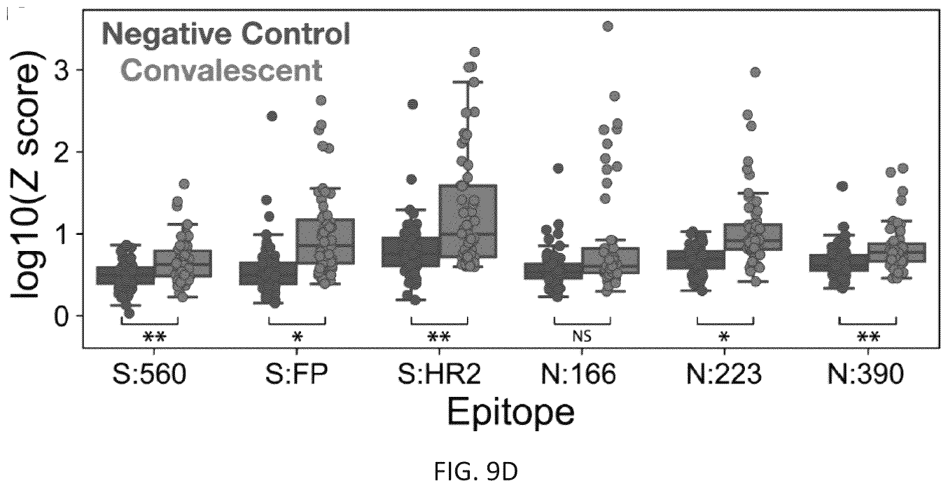

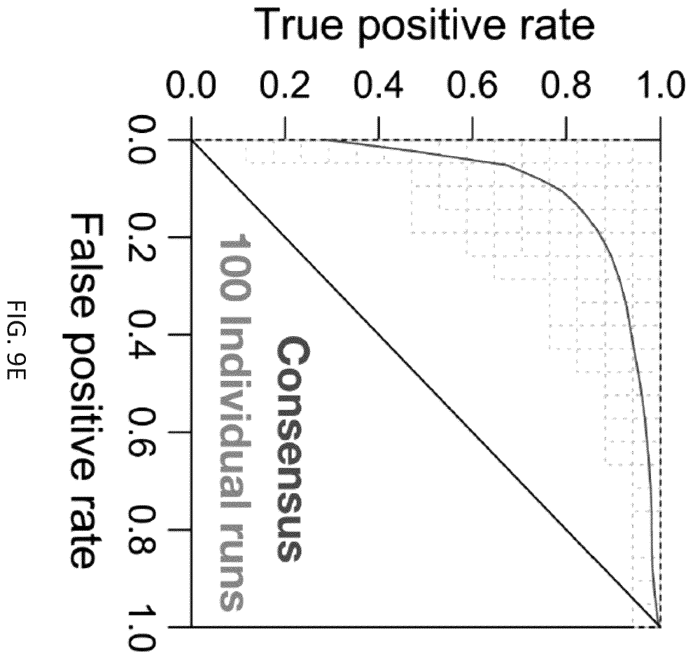

As expected, multiple positive control peptides were found to be enriched in every serum sample that we tested (FIG. 9A), and there was no significant difference between convalescent and negative samples in the number of enriched control peptides (t test, p=0.47). In contrast, we detected significantly more SARS-CoV-2 peptides enriched in convalescent samples compared to controls in both target proteins (t test; S: p=2.2e−7, 6.2-fold difference; N: p=1.9e−6, 15.7-fold difference) (FIG. 9A). We observed at least 1 enriched SARS-CoV-2 peptide from 50/55 convalescent samples (91%), with an average of 18 enriched peptides per sample; while enriched SARS-CoV-2 peptides were only observed in 25/68 (37%) negative samples, with an average of 2 enriched peptides per sample. For the convalescent donors, there was no correlation between the number of enriched control and SARS-CoV-2 peptides (p=0.94). Therefore, the absence of SARS-CoV-2 reactivity in some convalescent samples does not appear to be related to sample quality or a generally low concentration of IgG. We also did not observe a significant effect of gender in overall SARS-CoV-2 reactivity in convalescent donors (t test, p=0.56), nor a significant correlation between SARS-CoV-2 reactivity and the number of days between PCR diagnosis and sample collection (Pearson correlation=−0.13, p=0.35). Notably, however, the five convalescent donors without detectable SARS-CoV-2 peptide enrichment were well below the median age of the full convalescent donor population (22-43 versus 50). In fact, overall, we observed a significant positive correlation between age and the number of enriched SARS-CoV-2 peptides in convalescent donors (Pearson correlation=0.33, p=0.014), while a weak trend in the opposite direction was observed for the number of enriched control peptides (Pearson correlation=−0.21, p=0.13), indicating that this pattern was not due to an overall higher level of reactivity in samples from older patients.

In total, we identified IgG reactivity (i.e., peptide enrichment) against 229 and 95 SARS-CoV-2 peptides in convalescent and negative control samples, respectively; 70 of these peptides were enriched in both sample types. The peptides enriched in convalescent samples clustered together into 10 putative epitopes within the S protein and 9 putative epitopes within the N protein (FIGS. 9B and 9C; TABLE 4). These epitopes were recognized at a range of prevalences across the sampled population. The 6 most widely recognized epitopes—S positions 560-572, 819-824, and 1,150-1,156 and N positions 166-169, 223-229, and 390-402—were each detected in 13%-49% of the convalescent samples tested (median=28.2%, n=55), and all of the convalescent samples with at least 1 enriched SARS-CoV-2 peptide were reactive to at least 1 of these 6 immunodominant regions (FIGS. 9B and 9C). Notably, we also observed the enrichment of peptides from 4/6 of these immunodominant regions in negative control samples, although at much lower rates (1.5%-20% reactive, median=2.2%, n=68). At the other extreme, 9 (47%) of the observed epitope regions were each detected in only a single convalescent donor. Overall, relatively little reactivity was detected to peptides within the RBD, suggesting that these epitopes require protein conformations that are not well represented by linear 30-mer peptides.

To evaluate the potential for the highly recurrent S protein epitopes to be targeted by neutralizing antibodies, we evaluated these within the context of the structure of the protein. The inferred core regions (i.e., sequences present in all enriched peptides from assays of convalescent donors) of each of these epitopes were mapped onto a rendering of the three-dimensional structure of the native S trimer. All three epitope regions are accessible for antibody binding on the surface of the trimer. The most widely recognized region (1,150-1, 156) is located within the “stem helix” just upstream and partially overlapping with the heptad repeat region 2 (HR2). This region is proximal to the transmembrane domain and unresolved in the native structure; however, comparison of pre-and post-fusion structures indicated that the HR2 epitope lies within a region that undergoes a dramatic conformational rearrangement during fusion. The second epitope (819-824) resides near the S2′ cleavage site, spanning the fusion peptide (FP), whose exposure and incorporation into the host membrane are essential steps in virus entry into cells. Based on their proximity to these important functional sites, these epitopes are hereafter referred to as HR2 and FP, respectively. Finally, the 560-572 epitope occurs in the subdomain SDI region (in the S1 subunit but C-terminal of the RBD).

To explore the diagnostic potential of the six highly recurrent S and N epitopes, we compared the maximum Z scores per epitope across the full set of convalescent and negative samples (FIG. 9D). Across all six epitopes, we observed an overall shift toward higher Z scores in convalescent samples, which suggests the presence of additional antibody reactivity at these epitopes that is below our current enrichment thresholds. In fact, at 5/6 of these epitopes, we observed a significant difference in the mean of the Z score distributions between convalescent and negative samples (t test, S560: p=0.001, FP: p=0.036, HR2: p=0.008, N223: p=0.043, N390: p=0.008). To estimate the combined diagnostic performance of these six epitopes, we built logistic regression models using the maximum peptide Z score for each of the epitopes as features and the donor status (convalescent versus negative) as the predicted outcome. Cross-validated models each trained on a randomly selected subset of 70% of donors and tested on the remaining 30% gave a mean area under the curve of 0.92 (FIG. 9E).

FIGS. 9A-9E illustrate how PepSeq identified recurrent reactivities to SARS-CoV-2 peptides and classifies exposure status. FIG. 9A depicts boxplots showing the number of enriched SCV2 library peptides from assays with negative control (blue, n=68) and COVID-19 convalescent (orange, n=55) samples, divided into 3 different categories non-SARS-CoV-2 control peptides (Control), and SARS-CoV-2 Spike(S) and Nucleocapsid (N) peptides. ***t test with p<1e−5, NS, not significant). Individual data points are shown as circles, the limits of the boxes correspond to the 1st and 3rd quartiles, the black line inside each box corresponds to the median, and the whiskers extend to points that lie within 1.5 interquartile ranges of the 1st and 3rd quartiles

FIGS. 9B and 9C depict heatmaps showing the locations of enriched SARS-CoV-2 peptides within the S and N proteins, respectively Each row represents a single serum/plasma sample and each plot includes only samples with at least 1 enriched peptide from the focal protein. Each position is colored according to the number of enriched peptides that overlap that position. The horizontal dashed line separates COVID-19 convalescent samples (top) from negative control samples (bottom). The vertical dashed lines in FIG. 9B represent the S1-S2 and S2′ cleavage sites, respectively. The gray boxes indicate selected functional regions: receptor binding domain (RBD), fusion peptide (FP), and heptad repeat 2 (HR2).

FIG. 9D depicts boxplots showing the distribution of Z scores across all assayed samples for the 6 most common epitope reactivities observed in FIGS. 9B and 9C. For each sample/epitope combination, the Z score of the most enriched, overlapping peptide is presented. Boxplots were drawn as described for FIG. 9A, with convalescent samples in orange and negative controls in blue. t test: *p<0.05, **p<0.01, NS, not significant.

FIG. 9E depicts receiver-operating curves showing sensitivity/specificity across a range of thresholds with which logistic regression models trained on randomly selected subsets of 70% of the donors were able to classify the remaining 30% of donors as either negative control or convalescent, using log-transformed Z scores for the 6 epitopes described in FIG. 9D as features. The red curve shows the average of 100 individual runs Each patient sample was assayed in duplicate. Enriched peptides were determined based on consistent signal across replicates and Z scores shown as averages across replicates.

Additional experimental work by the inventors supporting the present invention is presented in Ladner J T, et al. Epitope-resolved profiling of the SARS-CoV-2 antibody response identifies cross-reactivity with endemic human coronaviruses. Cell Rep Med. 2021 Jan. 19; 2(1):100189. doi: 10.1016/j.xcrm.2020.100189. PMID: 33495758; PMCID: PMC7816965, which is hereby incorporated by reference.

TABLE 4
Putative epitope regions inferred using SCV2 PepSeq library.

| | | | | Enriched | Enriched | |
|---|---|---|---|---|---|---|
| | | | | COV-19 | Negative | SEQ |
| | First | Last | | Convalescent | Control | ID |
| Protein | Residue | Residue | Sequence* | Samples | Samples | NO: |
| Spike | 186 | 215 | FKNLREFVFKNIDGYFKIYSKHTPINLVRD | 1 | 0 | 424 |
| | 273 | 302 | RTFLLKYNENGTITDAVDCALDPLSETKCT | 0 | 1 | 425 |
| | 330 | 359 | PNITNLCPFGEVFNATRFASVYAWNRKRIS | 1 | 0 | 426 |
| | 402 | 431 | IRGDEVRQIAPGQTGKIADYNYKLPDDFTG | 0 | 1 | 417 |
| | 441 | 464 | LDSKVGGNYNYLYRLFRKSNLKPF | 2 | 2 | 428 |
| | 481 | 510 | NGVEGFNCYFPQSYGFQPTNGVGYQPYRV | 0 | 1 | 429 |
| | 532 | 559 | NLVKNKCVNFNFNGLTGTGVLTESNKKF | 0 | 1 | 430 |
| | 560 | 572 | LPFQQFGRRDIADT | 7 | 0 | 423 |
| | 619 | 647 | EVPVAIHADQLTPTWRVYSTGSNVFQTRA | 1 | 0 | 431 |
| | 647 | 668 | AGCLIGAEHVNNSYECDIPIGA | 1 | 0 | 432 |
| | 785 | 791 | VKQIYKT | 3 | 0 | 433 |
| | 819 | 824 | EDLLFN | 22 | 2 | 422 |
| | 901 | 930 | QMAYRFNGIGVTQNVLYENQKLIANQFNSA | 1 | 0 | 401 |
| | 939 | 968 | SSTASALGKLQDVVNQNAQALNTLVKQLSS | 0 | 1 | 434 |
| | 1150 | 1156 | EELDKYF | 27 | 14 | 435 |
| | 1155 | 1184 | YFKNHTSPDVDLGDISGINASVVNIQKEID | 1 | 1 | 436 |
| | 1184 | 1195 | DRLNEVAKNLNE | 0 | 4 | 437 |
| Nucleocapsid | 25 | 30 | GSNQNG | 3 | 2 | 438 |
| | 99 | 127 | GKMKDLSPRWYFYYLGTGPEAGLPYGANK | 1 | 0 | 439 |
| | 133 | 152 | VATEGALNTPKDHIGTRNPA | 1 | 1 | 440 |
| | 166 | 169 | TLPK | 12 | 3 | 441 |
| | 223 | 229 | LLDRLNQ | 19 | 0 | 442 |
| | 245 | 274 | TVTKKSAAEASKKPRQKRTATKAYNVTQAF | 1 | 0 | 443 |
| | 281 | 301 | QTQGNFGDQFLIRQGTDYKHW | 2 | 3 | 444 |
| | 346 | 361 | FKDQVILLNKHIDAYK | 2 | 0 | 445 |
| | 3643 | 375 | PKKDKKKK | 0 | 3 | 446 |
| | 390 | 402 | QTVTLLPAADLDD | 11 | 1 | 447 |

*Reported epitopes represent inferred minimally reactive regions based on enriched peptides across all samples. These regions may not represent the full epitope, due to slight differences in epitopes recognized across different individuals. All positions and sequences relative to GenBank: YP_009724390.1 (Spike) and YP_009724397.2 (Nucleocapsid).

Some embodiments of the invention may comprise the administration of a pharmaceutical composition to the subject that has been previously diagnosed with a viral infection, such as coronavirus infection. For example, in some embodiments, the subject may have been previously diagnosed with COVID-19 by one skilled in the art (e.g., a physician or a veterinarian) such that a therapeutic treatment is warranted by the diagnosis. Moreover, in other embodiments, the invention may comprise the administration of a pharmaceutical composition to a subject that may have not yet been exposed to a coronavirus-infected environment or individual. As such, the administration of the pharmaceutical composition may function as a vaccine or prophylactic agent to limit any potential viral infection that could occur, or to improve the subject's immune response to the virus.

All headings are for the convenience of the reader and should not be used to limit the meaning of the text that follows the heading, unless so specified.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

Amanat, F., Stadlbauer, D., Strohmeier, S., Nguyen, T. H. O., Chromikova, V., McMahon, M., Jiang, K., Arunkumar, G. A., Jurczyszak, D., Polanco, J., et al. (2020). A serological assay to detect SARS-CoV-2 seroconversion in humans. Nat. Med.
Callow, K. A., Parry, H. F., Sergeant, M., and Tyrrell, D. A. (1990). The time course of the immune response to experimental coronavirus infection of man. Epidemiol. Infect. 105, 435-446.
Casadevall, A., and Pirofski, L.-A. (2020). The convalescent sera option for containing COVID-19. J. Clin. Invest. 130, 1545-1548.
Chang, C.-K., Hsu, Y.-L., Chang, Y.-H., Chao, F.-A., Wu, M.-C., Huang, Y.-S., Hu, C.-K., and Huang, T.-H. (2009). Multiple nucleic acid binding sites and intrinsic disorder of severe acute respiratory syndrome coronavirus nucleocapsid protein: implications for ribonucleocapsid protein packaging. J. Virol. 83, 2255-2264.
Chi, X., Yan, R., Zhang, J., Zhang, G., Zhang, Y., Hao, M., Zhang, Z., Fan, P., Dong, Y., Yang, Y., et al. (2020). A neutralizing human antibody binds to the N-terminal domain of the Spike protein of SARS-CoV-2. Science.
Deeks, J. J., Dinnes, J., Takwoingi, Y., Davenport, C., Spijker, R., Taylor-Phillips, S., Adriano, A., Beese, S., Dretzke, J., Ferrante di Ruffano, L., et al. (2020). Antibody tests for identification of current and past infection with SARS-CoV-2. Cochrane Database Syst. Rev. 6, CD013652.
Dijkman, R., Jebbink, M. F., El Idrissi, N. B., Pyrc, K., Müller, M. A., Kuijpers, T. W., Zaaijer, H. L., and van der Hoek, L. (2008). Human coronavirus NL63 and 229E seroconversion in children. J. Clin. Microbiol. 46, 2368-2373.
Du, L., He, Y., Zhou, Y., Liu, S., Zheng, B.-J., and Jiang, S. (2009). The spike protein of SARS-CoV—a target for vaccine and therapeutic development. Nat. Rev. Microbiol. 7, 226-236.
Eroshenko, N., Gill, T., Keaveney, M. K., Church, G. M., Trevejo, J. M., and Rajaniemi, H. (2020). Implications of antibody-dependent enhancement of infection for SARS-CoV-2 countermeasures. Nat. Biotechnol.
Fink, Z. W., Martinez, V., Altin, J., and Ladner, J. T. (2020). PepSIRF: a flexible and comprehensive tool for the analysis of data from highly-multiplexed DNA-barcoded peptide assays. ar Xiv.
Fleri, W., Paul, S., Dhanda, S. K., Mahajan, S., Xu, X., Peters, B., and Sette, A. (2017). The Immune Epitope Database and Analysis Resource in Epitope Discovery and Synthetic Vaccine Design. Front. Immunol. 8, 278.
Friesen, R. H. E., Lee, P. S., Stoop, E. J. M., Hoffman, R. M. B., Ekiert, D. C., Bhabha, G., Yu, W., Juraszek, J., Koudstaal, W., Jongeneelen, M., et al. (2014). A common solution to group 2 influenza virus neutralization. Proc. Natl. Acad. Sci. U.S.A. 111, 445-450.
Gorse, G. J., Patel, G. B., Vitale, J. N., and O'Connor, T. Z. (2010). Prevalence of antibodies to four human coronaviruses is lower in nasal secretions than in serum. Clin. Vaccine Immunol. 17, 1875-1880.
Gostic, K. M., Ambrose, M., Worobey, M., and Lloyd-Smith, J. O. (2016). Potent protection against H5N1 and H7N9 influenza via childhood hemagglutinin imprinting. Science 354, 722-726.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501.e15.
Halstead, S. B., and O'rourke, E. J. (1977). Antibody-enhanced dengue virus infection in primate leukocytes. Nature 265, 739-741.
Hansen, J., Baum, A., Pascal, K. E., Russo, V., Giordano, S., Wloga, E., Fulton, B. O., Yan, Y., Koon, K., Patel, K., et al. (2020). Studies in humanized mice and convalescent humans yield a SARS-CoV-2 antibody cocktail. Science.
Hoofnagle, J. H., Gerety, R. J., Ni, L. Y., and Barker, L. F. (1974). Antibody to hepatitis B core antigen. A sensitive indicator of hepatitis B virus replication. N. Engl. J. Med. 290, 1336-1340.
Katzelnick, L. C., Gresh, L., Halloran, M. E., Mercado, J. C., Kuan, G., Gordon, A., Balmaseda, A., and Harris, E. (2017). Antibody-dependent enhancement of severe dengue disease in humans. Science 358, 929-932.
Keng, C.-T., Zhang, A., Shen, S., Lip, K.-M., Fielding, B. C., Tan, T. H. P., Chou, C.-F., Loh, C. B., Wang, S., Fu, J., et al. (2005). Amino acids 1055 to 1192 in the S2 region of severe acute respiratory syndrome coronavirus S protein induce neutralizing antibodies: implications for the development of vaccines and antiviral agents. J. Virol. 79, 3289-3296.
Khan, S., Nakajima, R., Jain, A., de Assis, R. R., Jasinskas, A., Obiero, J. M., Adenaiye, O., Tai, S., Hong, F., Milton, D. K., et al. (2020). Analysis of Serologic Cross-Reactivity Between Common Human Coronaviruses and SARS-CoV-2 Using Coronavirus Antigen Microarray. bioRxiv.
Khurana, S., Loving, C. L., Manischewitz, J., King, L. R., Gauger, P. C., Henningson, J., Vincent, A. L., and Golding, H. (2013). Vaccine-induced anti-HA2 antibodies promote virus fusion and enhance influenza virus respiratory disease. Sci. Transl. Med. 5, 200ra114.
Kozlov, I. A., Thomsen, E. R., Munchel, S. E., Villegas, P., Capek, P., Gower, A. J., Pond, S. J. K., Chudin, E., and Chee, M. S. (2012). A highly scalable peptide-based assay system for proteomics. PLOS One 7, e37441.
Krammer, F., and Simon, V. (2020). Serology assays to manage COVID-19. Science 368, 1060-1061.
Lai, S.-C., Chong, P. C.-S., Yeh, C.-T., Liu, L. S.-J., Jan, J.-T., Chi, H.-Y., Liu, H.-W., Chen, A., and Wang, Y.-C. (2005). Characterization of neutralizing monoclonal antibodies recognizing a 15-residues epitope on the spike protein HR2 region of severe acute respiratory syndrome coronavirus (SARS-CoV). J. Biomed. Sci. 12, 711-727.
Larman, H. B., Zhao, Z., Laserson, U., Li, M. Z., Ciccia, A., Gakidis, M. A. M., Church, G. M., Kesari, S., Leproust, E. M., Solimini, N. L., et al. (2011). Autoantigen discovery with a synthetic human peptidome. Nat. Biotechnol. 29, 535-541.
Liu, A., Li, Y., Peng, J., Huang, Y., and Xu, D. (2020). Antibody responses against SARS-CoV-2 in COVID-19 patients. J. Med. Virol.
Liu, S., Xiao, G., Chen, Y., He, Y., Niu, J., Escalante, C. R., Xiong, H., Farmar, J., Debnath, A. K., Tien, P., et al. (2004). Interaction between heptad repeat 1 and 2 regions in spike protein of SARS-associated coronavirus: implications for virus fusogenic mechanism and identification of fusion inhibitors. Lancet 363, 938-947.
Lu, R., Zhao, X., Li, J., Niu, P., Yang, B., Wu, H., Wang, W., Song, H., Huang, B., Zhu, N., et al. (2020). Genomic characterisation and epidemiology of 2019 novel coronavirus: implications for virus origins and receptor binding. Lancet 395, 565-574.
Lubroth, J., Grubman, M. J., Burrage, T. G., Newman, J. F., and Brown, F. (1996). Absence of protein 2C from clarified foot-and-mouth disease virus vaccines provides the basis for distinguishing convalescent from vaccinated animals. Vaccine 14, 419-427.
Lucchese, G., Stufano, A., Trost, B., Kusalik, A., and Kanduc, D. (2007). Peptidology: short amino acid modules in cell biology and immunology. Amino Acids 33, 703-707.
Lv, H., Wu, N. C., Tsang, O. T.-Y., Yuan, M., Perera, R. A. P. M., Leung, W. S., So, R. T. Y., Chan, J. M. C., Yip, G. K., Chik, T. S. H., et al. (2020). Cross-reactive Antibody Response between SARS-CoV-2 and SARS-CoV Infections. Cell Rep. 31, 107725.
Mina, M. J., Kula, T., Leng, Y., Li, M., de Vries, R. D., Knip, M., Siljander, H., Rewers, M., Choy, D. F., Wilson, M. S., et al. (2019). Measles virus infection diminishes preexisting antibodies that offer protection from other pathogens. Science 366, 599-606.
Monto, A. S., Malosh, R. E., Petrie, J. G., and Martin, E. T. (2017). The Doctrine of Original Antigenic Sin: Separating Good From Evil. J. Infect. Dis. 215, 1782-1788.
Ni, L., Ye, F., Cheng, M.-L., Feng, Y., Deng, Y.-Q., Zhao, H., Wei, P., Ge, J., Gou, M., Li, X., et al. (2020). Detection of SARS-CoV-2-Specific Humoral and Cellular Immunity in COVID-19 Convalescent Individuals. Immunity 52, 971-977.e3.
Nie, J., Li, Q., Wu, J., Zhao, C., Hao, H., Liu, H., Zhang, L., Nie, L., Qin, H., Wang, M., et al. (2020). Establishment and validation of a pseudovirus neutralization assay for SARS-CoV-2. Emerg. Microbes Infect. 9, 680-686.
Pillay, T. S. (2020). Gene of the month: the 2019-nCOV/SARS-CoV-2 novel coronavirus spike protein. J. Clin. Pathol. 73, 366-369.
Pinto, D., Park, Y.-J., Beltramello, M., Walls, A. C., Tortorici, M. A., Bianchi, S., Jaconi, S., Culap, K., Zatta, F., De Marco, A., et al. (2020). Cross-neutralization of SARS-CoV-2 by a human monoclonal SARS-CoV antibody. Nature.
Poh, C. M., Carissimo, G., Wang, B., Amrun, S. N., Lee, C. Y.-P., Chee, R. S.-L., Fong, S.-W., Yeo, N. K.-W., Lee, W.-H., Torres-Ruesta, A., et al. (2020). Two linear epitopes on the SARS-CoV-2 spike protein that elicit neutralising antibodies in COVID-19 patients. Nat. Commun. 11, 2806.
Price, J. V., Tangsombatvisit, S., Xu, G., Yu, J., Levy, D., Baechler, E. C., Gozani, O., Varma, M., Utz, P. J., and Liu, C. L. (2012). On silico peptide microarrays for high-resolution mapping of antibody epitopes and diverse protein-protein interactions. Nat. Med. 18, 1434-1440.
Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent Antibody Responses to SARS-CoV-2 Infection in Convalescent Individuals. bioRxiv.
Routledge, E., Stauber, R., Pfleiderer, M., and Siddell, S. G. (1991). Analysis of murine coronavirus surface glycoprotein functions by using monoclonal antibodies. J. Virol. 65, 254-262.
Shiryaev, S. A., Thomsen, E. R., Cieplak, P., Chudin, E., Cheltsov, A. V., Chee, M. S., Kozlov, I. A., and Strongin, A. Y. (2012). New details of HCV NS3/4A proteinase functionality revealed by a high-throughput cleavage assay. PLOS One 7, e35759.
Tan, C. W., Chia, W. N., Chen, M. I.-C., Hu, Z., Young, B. E., Tan, Y.-J., Yi, Y., Lye, D. C., Anderson, D. E., and Wang, L.-F. A SARS-CoV-2 surrogate virus neutralization test (sVNT) based on antibody-mediated blockage of ACE2-spike (RBD) protein-protein interaction.
Thanh Le, T., Andreadakis, Z., Kumar, A., Gómez Román, R., Tollefsen, S., Saville, M., and Mayhew, S. (2020). The COVID-19 vaccine development landscape. Nat. Rev. Drug Discov. 19, 305-306.
Walls, A. C., Tortorici, M. A., Snijder, J., Xiong, X., Bosch, B.-J., Rey, F. A., and Veesler, D. (2017). Tectonic conformational changes of a coronavirus spike glycoprotein promote membrane fusion. Proc. Natl. Acad. Sci. U.S.A. 114, 11157-11162.
Waterhouse, A., Bertoni, M., Bienert, S., Studer, G., Tauriello, G., Gumienny, R., Heer, F. T., de Beer, T. A. P., Rempfer, C., Bordoli, L., et al. (2018). SWISS-MODEL: homology modelling of protein structures and complexes. Nucleic Acids Res. 46, W296-W303.
Whitman, J. D., Hiatt, J., Mowery, C. T., Shy, B. R., Yu, R., Yamamoto, T. N., Rathore, U., Goldgof, G. M., Whitty, C., Woo, J. M., et al. (2020). Test performance evaluation of SARS-CoV-2 serological assays. medRxiv.
Xia, S., Yan, L., Xu, W., Agrawal, A. S., Algaissi, A., Tseng, C.-T. K., Wang, Q., Du, L., Tan, W., Wilson, I. A., et al. (2019). A pan-coronavirus fusion inhibitor targeting the HR1 domain of human coronavirus spike. Sci Adv 5, eaav4580.
Xu, G. J., Kula, T., Xu, Q., Li, M. Z., Vernon, S. D., Ndung'u, T., Ruxrungtham, K., Sanchez, J., Brander, C., Chung, R. T., et al. (2015). Viral immunology. Comprehensive serological profiling of human populations using a synthetic human virome. Science 348, aaa0698.
Yuan, M., Wu, N. C., Zhu, X., Lee, C.-C. D., So, R. T. Y., Lv, H., Mok, C. K. P., and Wilson, I. A. A highly conserved cryptic epitope in the receptor-binding domains of SARS-CoV-2 and SARS-CoV.
Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N. Engl. J. Med. 382, 727-733.
Zost, S. J., Gilchuk, P., Case, J. B., Binshtein, E., Chen, R. E., Reidy, J. X., Trivette, A., Nargi, R. S., Sutton, R. E., Suryadevara, N., et al. (2020). Potently neutralizing human antibodies that block SARS-CoV-2 receptor binding and protect animals. bioRxiv.
Centers for Disease Control and Prevention (CDC) (2003). Revised U.S. surveillance case definition for severe acute respiratory syndrome (SARS) and update on SARS cases—United States and worldwide, December 2003. MMWR Morb. Mortal. Wkly. Rep. 52, 1202-1206.
Jia, N., Liu, H.-B., Ni, X.-B., Bell-Sakyi, L., Zheng, Y.-C., Song, J.-L., Li, J., Jiang, B.-G., Wang, Q., Sun, Y., et al. (2019). Emergence of human infection with Jingmen tick virus in China: A retrospective study. EBioMedicine.
Woolhouse, M. E. J., and Brierley, L. (2018). Epidemiological characteristics of human-infective RNA viruses. Sci Data 5, 180017.

1-20. (canceled)

21. A method of detecting in a sample a presence of an antibody that binds to a spike protein or a nucleocapsid protein of a severe acute respiratory syndrome-associated coronavirus (SARS-CoV), the method comprising:
providing a biological sample from a subject suspected to be infected with a SARS-CoV;
contacting the biological sample with a peptide consisting of the amino acid sequence: FKEELDKYF (SEQ ID NO: 421); and
detecting antigen-antibody complexes formed.

22. The method of claim 21, wherein the SARS-CoV is severe acute respiratory syndrome coronavirus 2 (SARS-CoV-2).

23. The method of claim 21, wherein the biological sample is whole blood, serum, or plasma.

24. The method of claim 21, wherein the subject is a human.

25. The method of claim 21, wherein the method of detecting antigen-antibody complexes comprises a technique selected from the group consisting of flow cytometry, immunohistochemistry, enzyme-linked immunosorbent assay (ELISA), Western Blot, and immunoaffinity chromatography.

26. The method of claim 21, further comprising contacting the biological sample with one or more peptides selected from the group consisting of SEQ ID NOs: 1-4, 275-412, and 422-447; and detecting antigen-antibody complexes formed.

27. The method of claim 26, wherein the one or more peptides is selected from the group consisting of SEQ ID NO: 422, SEQ ID NO: 423, SEQ ID NO: 435, SEQ ID NO: 441, SEQ ID NO: 442; and SEQ ID NO: 447.

28. The method of claim 26, wherein the one or more peptides is selected from the group consisting of SEQ ID NO: 1, SEQ ID NO: 2, SEQ ID NO: 3, and SEQ ID NO: 4.

29. An immunogenic composition, comprising:
one or more peptides consisting of an amino acid sequence selected from the group consisting of SEQ ID NOs: 1-4, 275-412, and 421-423; and
a pharmaceutically acceptable carrier or adjuvant.

30. The immunogenic composition of claim 29, wherein the immunogenic composition consists of one or more peptides selected from the group consisting of SEQ ID NO: 1, SEQ ID NO: 2, SEQ ID NO: 3, and SEQ ID NO: 4.

31. The immunogenic composition of claim 29, wherein the immunogenic composition consists of one or more peptides selected from the group consisting of SEQ ID NO: 421, SEQ ID NO: 422, SEQ ID NO: 423, SEQ ID NO: 435, SEQ ID NO: 441, SEQ ID NO: 442; and SEQ ID NO: 447.

32. A method of eliciting an immune response to a spike protein or a nucleocapsid protein of a SARS-CoV in a subject, the method comprising administering to the subject the immunogenic composition of claim 29.

33. The method of claim 32, wherein the immunogenic composition is administered to the subject prophylactically.

32. The method of claim 32, wherein the subject is a human.

35. The method of claim 32, wherein the SARS-CoV is severe acute respiratory syndrome coronavirus 2 (SARS-CoV-2).

36. A peptide-based diagnostic kit for detecting SARS-CoV in a subject comprising one or more peptides consisting of an amino acid sequence selected from the group consisting of SEQ ID NOs: 1-4, 275-412, and 421-423.

37. The peptide-based diagnostic kit of claim 36, wherein the kit consists of one or more peptides selected from the group consisting of SEQ ID NO: 1, SEQ ID NO: 2, SEQ ID NO: 3, and SEQ ID NO: 4.

38. The peptide-based diagnostic kit of claim 36, wherein the kit consists of one or more peptides selected from the group consisting of SEQ ID NO: 421, SEQ ID NO: 422, SEQ ID NO: 423, SEQ ID NO: 435, SEQ ID NO: 441, SEQ ID NO: 442; and SEQ ID NO: 447.

39. The peptide-based diagnostic kit of claim 36, wherein the SARS-CoV is severe acute respiratory syndrome coronavirus 2 (SARS-CoV-2).


SEQUENCE LISTING (464 sequences)

SEQ ID NO: 1 - protein, 30 aa, Severe acute respiratory syndrome coronavirus 2
SFKEELDKYFKNHTSPDVDLGDISGINASV

SEQ ID NO: 2 - protein, 30 aa, Severe acute respiratory syndrome coronavirus 2
SKPSKRSFIEDLLFNKVTLADAGFIKQYGD

SEQ ID NO: 3 - protein, 30 aa, Severe acute respiratory syndrome coronavirus 2
NAAIVLQLPQGTTLPKGFYAEGSRGGSQAS

SEQ ID NO: 4 - protein, 30 aa, Severe acute respiratory syndrome coronavirus 2
GDAALALLLLDRLNQLESKMSGKGQQQQGQ

SEQ ID NO: 5 - protein, 30 aa, Aichivirus A
SLIKRQGNRVIDAEPREIPLEYADDLLEAM

SEQ ID NO: 6 - protein, 30 aa, Aichivirus A
EACWKCSQDKPRRKYNTVPPEEWLYDSDVQ

SEQ ID NO: 7 - protein, 30 aa, Aichivirus A
ALPGIRRQGLLTLSADTETNQTLNKITESV

SEQ ID NO: 8 - protein, 30 aa, Aichivirus A
KFFDKLALLSLPGAYQAKTPEERALAGALT

SEQ ID NO: 9 - protein, 30 aa, Aichivirus A
TQSGNAAILTGSTAPSFLAYPTATPVPLPN

SEQ ID NO: 10 - protein, 30 aa, Aichivirus A
GSSNKVGSRFSKWWEPAAARALERATDSAI

SEQ ID NO: 11 - protein, 30 aa, Alphacoronavirus 1
MATQGPRVNWGDEPSKRRGRSNSRGRKSSD

SEQ ID NO: 12 - protein, 30 aa, Alphapapillomavirus 9
MSLWRPSEATVYLPPAPVSKVVSTDEYVTR

SEQ ID NO: 13 - protein, 30 aa, Betacoronavirus 1
TTGYRFTNFEPFTVNSVNDSLEPVGGLYEI

SEQ ID NO: 14 - protein, 30 aa, Betacoronavirus 1
APDVMLNISTPKLPDFKEELDQWFKNQTSV

SEQ ID NO: 15 - protein, 30 aa, Betacoronavirus 1
KWADQSDQFRNVQTRGRRAQPKQTVTSQQP

SEQ ID NO: 16 - protein, 30 aa, Enterovirus A
AANTAASAHSLGTGRVPALQAAETGASSNS

SEQ ID NO: 17 - protein, 30 aa, Enterovirus A
VSDYIKGLGDAFGVGFTDAVSREVEALKNH

SEQ ID NO: 18 - protein, 30 aa, Enterovirus A
DSIADMIDQAVNNQVNRSLTAMQVLPTAAN

SEQ ID NO: 19 - protein, 30 aa, Enterovirus A
DMANAAKGFEWISNKISKFIDWIKEKIIPA

SEQ ID NO: 20 - protein, 30 aa, Enterovirus A
PPKFRPVRISLDEKPAPDAISDLLASVDSE

SEQ ID NO: 21 - protein, 30 aa, Enterovirus A
NLEAIDLHTSAGYPYSALGIKKRDILDPTT

SEQ ID NO: 22 - protein, 30 aa, Enterovirus A
DKRLEVDFETALFSKYIGNKIYEPDEYMIQ

SEQ ID NO: 23 - protein, 30 aa, Enterovirus A
TAEYQNDPITNAVENAVSALADTTISRVTA

SEQ ID NO: 24 - protein, 30 aa, Enterovirus A
VSKMKFYMDKYGLDLPYSTYVKDELRSMDK

SEQ ID NO: 25 - protein, 30 aa, Enterovirus A
FTNINYYKDSYAASAAKHDFTQDPGKFTQP

SEQ ID NO: 26 - protein, 30 aa, Enterovirus A
SNKETGRLSINGPTRTKLEPSAFYDVFEGS

SEQ ID NO: 27 - protein, 30 aa, Enterovirus B
QQVPALTAVETGHTSQVAPSDTIQTRHVHN

SEQ ID NO: 28 - protein, 30 aa, Enterovirus B
VEGAIGRVADTIRSGPSNSEAVPALTAAET

SEQ ID NO: 29 - protein, 30 aa, Enterovirus B
KDAGYPVINAPSKTKLEPSVFHQVFEGNKE

SEQ ID NO: 30 - protein, 30 aa, Enterovirus B
NRQDFTQDPSRFTEPVQDVLIKTLPALNSP

SEQ ID NO: 31 - protein, 30 aa, Enterovirus B
ALYQNDPESALNRAVGRVADTVASGPVNTE

SEQ ID NO: 32 - protein, 30 aa, Enterovirus B
ASEVTVSDTQPSGPSNSVSVPMLTAAETGH

SEQ ID NO: 33 - protein, 30 aa, Enterovirus B
DVVEAIESAVARVADTISSGPTNSQAVPAL

SEQ ID NO: 34 - protein, 30 aa, Enterovirus B
SPFVRQTVFYQNDVQNAIDRAMTRVADTMP

SEQ ID NO: 35 - protein, 30 aa, Enterovirus B
NEPSNAIERAMVRVADTMASGPANSEQIPA

SEQ ID NO: 36 - protein, 30 aa, Enterovirus B
SQDNKLQGDVEEAIERAVVHVADTMRSGPS

SEQ ID NO: 37 - protein, 30 aa, Enterovirus D
ITDYIQSLGNAFGAGFTETISSKAKEVQDM

SEQ ID NO: 38 - protein, 30 aa, Enterovirus D
IIKTATDTVKSEINAELGVVPSLNAVETGA

SEQ ID NO: 39 - protein, 30 aa, Enterovirus D
KDKRLKVDFEEAIFSKYVGNKTMLMDEYME

SEQ ID NO: 40 - protein, 30 aa, Enterovirus D
GVIPSLNAAETGATSNTTPEEAIQTRAVIN

SEQ ID NO: 41 - protein, 30 aa, Enterovirus D
RDIFNRQTRDTTEMTKMLDKYGVDLPFVTF

SEQ ID NO: 42 - protein, 30 aa, Enterovirus D
RGKERAPNALNAIIGNRDSVKAMPHNIVTT

SEQ ID NO: 43 - protein, 30 aa, Enterovirus D
LFQGPPQFREIKISVSPETPAPDAINDLLR

SEQ ID NO: 44 - protein, 30 aa, Enterovirus D
ANYKGKEKTPNALNALIGNRDNVKTMTHNI

SEQ ID NO: 45 - protein, 30 aa, Enterovirus E
RRYFTAEQGKIEYIEKSKEAGYPIINAPTQ

SEQ ID NO: 46 - protein, 30 aa, Enterovirus E
KFTEAVNAFKGLDWIAAKFSKFLDWIKSKI

SEQ ID NO: 47 - protein, 30 aa, Enterovirus E
SVDSEDVREYCRQKGWIVQEKITKESLERN

SEQ ID NO: 48 - protein, 30 aa, Human alphaherpesvirus 1
WMRRRTQKAPKRIRLPHIREDDQPSAHQPL

SEQ ID NO: 49 - protein, 30 aa, Human alphaherpesvirus 1
GDFDEAKLAEAREMIRYMALVSAMEHTEHK

SEQ ID NO: 50 - protein, 30 aa, Human alphaherpesvirus 1
PLDGCGPLHPSWVSLMPPKQVPETVVDAAC

SEQ ID NO: 51 - protein, 30 aa, Human alphaherpesvirus 1
AWGQVHDWTEQTDPWFLDGLGMDRMYWRDT

SEQ ID NO: 52 - protein, 30 aa, Human alphaherpesvirus 1
SGTPAPAPPGDGSYLWIPASHYNQLVAGHA

SEQ ID NO: 53 - protein, 30 aa, Human alphaherpesvirus 1
PSTQTRAPLPTEPAFPPAATGSQPEASNAE

SEQ ID NO: 54 - protein, 30 aa, Human alphaherpesvirus 1
HRPAPGSPPGIPEYAEDPYAISYGGQLDRY

SEQ ID NO: 55 - protein, 30 aa, Human alphaherpesvirus 1
AAPASPYIEAENPLYDWGGSALFSPPGRTG

SEQ ID NO: 56 - protein, 30 aa, Human alphaherpesvirus 1
WHIPSIQDAATPYHPPATPNNMGLIAGAVG

SEQ ID NO: 57 - protein, 30 aa, Human alphaherpesvirus 1
RSMAESDVVMEDVAIAERALGLSAFGVAGG

SEQ ID NO: 58 - protein, 30 aa, Human alphaherpesvirus 1
ASGKGPTYIRVADSELYADWSSDSEGERDQ

SEQ ID NO: 59 - protein, 30 aa, Human alphaherpesvirus 2
FWVRRRAQMAPKRLRLPHIRDDDAPPSHQP

SEQ ID NO: 60 - protein, 30 aa, Human alphaherpesvirus 2
EAYYSESEDEAANDFLVRMGRQQSVLRRRR

SEQ ID NO: 61 - protein, 30 aa, Human alphaherpesvirus 2
DQTLQLHREGVSTQDPRFVGAFMAAKAAHL

SEQ ID NO: 62 - protein, 30 aa, Human alphaherpesvirus 2
PSEAVRPSRIPRAPRVPREPRVPREPREPR

SEQ ID NO: 63 - protein, 30 aa, Human alphaherpesvirus 2
ASGPHETITALVGAVTSLQQELAHMRARTN

SEQ ID NO: 64 - protein, 30 aa, Human alphaherpesvirus 2
RRRHEVEQPEYDCGRDEPDRDFPYYPGEAR

SEQ ID NO: 65 - protein, 30 aa, Human alphaherpesvirus 2
KKGTSALLSSKVTNMVLRKRNKARYSPLHN

SEQ ID NO: 66 - protein, 30 aa, Human alphaherpesvirus 2
AGVYDAVRTWGPDAEAEPDQMENTYLLPDD

SEQ ID NO: 67 - protein, 30 aa, Human alphaherpesvirus 2
APHAWGMLNDMQWLASSDSEEETEVGISDD

SEQ ID NO: 68 - protein, 30 aa, Human alphaherpesvirus 2
DRDSSMSLADFHGEEFEKLYEHLEAMGFGE

SEQ ID NO: 69 - protein, 30 aa, Human alphaherpesvirus 2
DPIYDEVAPDHEAELYARVQRPGPVPDAEP

SEQ ID NO: 70 - protein, 30 aa, Human alphaherpesvirus 3
LENAHEHHGVYNQGRGIDSGERLMQPTQMS

SEQ ID NO: 71 - protein, 30 aa, Human alphaherpesvirus 3
AVAPTSAATRKPDPAVAPTSAASRKPDPAV

SEQ ID NO: 72 - protein, 30 aa, Human alphaherpesvirus 3
KGLKQLPEGMDPFAEKPNATDTPIEEIGDS

SEQ ID NO: 73 - protein, 30 aa, Human alphaherpesvirus 3
DKFREAQEMIKYMTLVSAAERQESKARKKN

SEQ ID NO: 74 - protein, 30 aa, Human alphaherpesvirus 3
HAESSWVNRGESSRKAYDHNSPYIWPRNDY

SEQ ID NO: 75 - protein, 30 aa, Human alphaherpesvirus 3
VVTTETKSVVKEGIENHVYPTDMSTLPEKS

SEQ ID NO: 76 - protein, 30 aa, Human alphaherpesvirus 3
HQPNDSSGSEDDFEDIDEVVAAFREARLRH

SEQ ID NO: 77 - protein, 30 aa, Human alphaherpesvirus 3
EPMYAQVRKPKSRTDTQTTGRITNRSRARS

SEQ ID NO: 78 - protein, 30 aa, Human alphaherpesvirus 3
IPADEEAPTTPEDPRHPLHAHQLVPNSLNV

SEQ ID NO: 79 - protein, 30 aa, Human betaherpesvirus 5
APTPTFAGTQTPVNGNSPWAPTAPLPGDMN

SEQ ID NO: 80 - protein, 30 aa, Human betaherpesvirus 5
PANWPRERAWALKNPHLAYNPFRMPTTSTA

SEQ ID NO: 81 - protein, 30 aa, Human betaherpesvirus 5
STPRAAVTQTASQNAADEVWALRDQTAESP

SEQ ID NO: 82 - protein, 30 aa, Human betaherpesvirus 5
VKYQALATASGEEVAALSHHDSLESRRLRE

SEQ ID NO: 83 - protein, 30 aa, Human betaherpesvirus 5
ASEALDPHAFHLLLNTYGRPIRLLRENTTQ

SEQ ID NO: 84 - protein, 30 aa, Human betaherpesvirus 5
PSLKPTLGGKAVVGRPPSVPVSGSAPGRLS

SEQ ID NO: 85 - protein, 30 aa, Human betaherpesvirus 5
QQNGTDSLDGRTGTQDKGQKPNLLDRLRHR

SEQ ID NO: 86 - protein, 30 aa, Human betaherpesvirus 5
EQPTETPPEDLDTLSLAIEAAIQDLRNKSQ

SEQ ID NO: 87 - protein, 30 aa, Human betaherpesvirus 5
ASTTPTYPAVTTVYPPSSTAKSSVSNAPPV

SEQ ID NO: 88 - protein, 30 aa, Human betaherpesvirus 5
ALYMGSRRIPRKPRYTRLPKHDPDEFWTKT

SEQ ID NO: 89 - protein, 30 aa, Human betaherpesvirus 5
GRGSPLTIESHLSDNEEDPIRYYVSVYDEL

SEQ ID NO: 90 - protein, 30 aa, Human betaherpesvirus 6
SDPLEAFKTVNRHNWSDEQREHFYDLRNLY

SEQ ID NO: 91 - protein, 30 aa, Human betaherpesvirus 6
RKQKKLDLLGSWTKEKNDKAIVHSREVTGD

SEQ ID NO: 92 - protein, 30 aa, Human betaherpesvirus 6
NTAANADVFDPVHRLVSEQTGTPFVLNNSD

SEQ ID NO: 93 - protein, 30 aa, Human betaherpesvirus 6
PESDSVDNAGGKILIKKETLGGRDVRATTP

SEQ ID NO: 94 - protein, 30 aa, Human betaherpesvirus 6
KLPGNGEREIDLALQKVKAGERETSDFKVG

SEQ ID NO: 95 - protein, 30 aa, Human betaherpesvirus 6
GDEYSQEDALKMLKAIKSLDESYRRKPSSS

SEQ ID NO: 96 - protein, 30 aa, Human betaherpesvirus 6
EISDNIYSSPKNSIYLKSKSQQSTTKFTDT

SEQ ID NO: 97 - protein, 30 aa, Human betaherpesvirus 6
TTHSTETGVSPHNVSLIKDLRDKDGFRKQK

SEQ ID NO: 98 - protein, 30 aa, Human betaherpesvirus 6
VQSPFRLPNADLSRDLDSASFKDALDLKLP

SEQ ID NO: 99 - protein, 30 aa, Human betaherpesvirus 6
KEKRKVEDIDKKKEDEKRKQEEKKRNDEDK

SEQ ID NO: 100 - protein, 30 aa, Human betaherpesvirus 7
LKNLLNSRKRDPLFQNFSFTEKMQPVRSPF

SEQ ID NO: 101 - protein, 30 aa, Human betaherpesvirus 7
QLVKDVKWTPSSSLLDLSRRNDLLQKELFE

SEQ ID NO: 102 - protein, 30 aa, Human betaherpesvirus 7
NKIDYHSTFFLPENEVNRQNGVQSRDQLSK

SEQ ID NO: 103 - protein, 30 aa, Human betaherpesvirus 7
DLIDLENSVQKDDDIVNKLVSHLTHSEEDV

SEQ ID NO: 104 - protein, 30 aa, Human betaherpesvirus 7
QDFDSGSLLTGKETQNTIFGASKAQENGDK

SEQ ID NO: 105 - protein, 30 aa, Human betaherpesvirus 7
ATPIERSSRSASIISGESVPGFFNDQERLS

SEQ ID NO: 106 - protein, 30 aa, Human betaherpesvirus 7
RRKRELETNKDIVYVQLQYLYDTLKDYINT

SEQ ID NO: 107 - protein, 30 aa, Human betaherpesvirus 7
PVLNISRPGSTTPSGNSARYGNNTPRSITP

SEQ ID NO: 108 - protein, 30 aa, Human betaherpesvirus 7
NSVLQATQSVQAQVKEPLDSSPPYLKTNKD

SEQ ID NO: 109 - protein, 30 aa, Human betaherpesvirus 7
KRRKEIVHENLQSFDDEHNEMSLPPQDQKS

SEQ ID NO: 110 - protein, 30 aa, Human coronavirus 229E
YTVPDLVVEQYNQTILNLTSEISTRENKSA

SEQ ID NO: 111 - protein, 30 aa, Human coronavirus 229E
SDRNHNSQDDIMKAVAAALKSLGFDKPQEK

SEQ ID NO: 112 - protein, 30 aa, Human coronavirus 229E
EFNPSQTSPATVEPVRDEVSIETDIIDEVN

SEQ ID NO: 113 - protein, 30 aa, Human coronavirus 229E
GAMLSENFTSYGFSNVVEMPKFFYASNGTY

SEQ ID NO: 114 - protein, 30 aa, Human coronavirus 229E
SNQDDIMAAVAAALEKLGFERPNDASQPQK

SEQ ID NO: 115 - protein, 30 aa, Human coronavirus HKU1
DYALPSSRRKRRGISSPYRFVTFEPFNVSF

SEQ ID NO: 116 - protein, 30 aa, Human coronavirus HKU1
KPDMADEIASLVLAKLGKDSKPQQVTKQNA

SEQ ID NO: 117 - protein, 30 aa, Human coronavirus NL63
FGLQDGFYSANFLDDNVLPETYVALPIYYQ

SEQ ID NO: 118 - protein, 30 aa, Human coronavirus NL63
NTVLNASIPESKPLADDDSAIIEIVNEVLH

SEQ ID NO: 119 - protein, 30 aa, Human coronavirus NL63
ERWRMRRGQRVDLPPKVHFYYLGTGPHKDL

SEQ ID NO: 120 - protein, 30 aa, Human coronavirus NL63
VSNGGNNCTTAVMTYSNFGICADGSLIPVR

SEQ ID NO: 121 - protein, 30 aa, Human gammaherpesvirus 4
PGRRPFFHPVGQADYFEYHQEGGPDGEPDM

SEQ ID NO: 122 - protein, 30 aa, Human gammaherpesvirus 4
SPSLPSSKKGADEFEAWLEAQDANFEDVQR

SEQ ID NO: 123 - protein, 30 aa, Human gammaherpesvirus 4
MMDPNSTSEDVKFTPDPYQVPFVQAFDQAT

SEQ ID NO: 124 - protein, 30 aa, Human gammaherpesvirus 4
QHASGEGPGINPISKTELQAIMLALHEQNQ

SEQ ID NO: 125 - protein, 30 aa, Human gammaherpesvirus 4
EQEYGDKEVKLPHWTPTLHTFQVPKNYTKA

SEQ ID NO: 126 - protein, 30 aa, Human gammaherpesvirus 4
GPRHRDGVRRPQKRPSCIGCKGAHGGTGTG

SEQ ID NO: 127 - protein, 30 aa, Human gammaherpesvirus 4
QPMEGPLVPEQWMFPGAALSQRVRPGVAQS

SEQ ID NO: 128 - protein, 30 aa, Human gammaherpesvirus 4
MHPLTHQSIPNDPDSPEPRSPTVFYNIPPM

SEQ ID NO: 129 - protein, 30 aa, Human gammaherpesvirus 4
LPPRVRGGGRVSAAAITWVPKPNVEVWPVD

SEQ ID NO: 130 - protein, 30 aa, Human gammaherpesvirus 4
RKPGGPWRPEPNTSSPSMPELSPVLGLHQG

SEQ ID NO: 131 - protein, 30 aa, Human gammaherpesvirus 4
SPIHEPESHNSPEAPILFPDDWYPPSIDPA

SEQ ID NO: 132 - protein, 30 aa, Human mastadenovirus A
LAAPRRGNVYWVRDAVTGTRVPVRTRPPHP

SEQ ID NO: 133 - protein, 30 aa, Human mastadenovirus A
IESLVDKMSRWKTYAQERHEWEERQPKPVP

SEQ ID NO: 134 - protein, 30 aa, Human mastadenovirus A
EENDDFNPVYPFDPYDTAHVPFVTPPFTSS

SEQ ID NO: 135 - protein, 30 aa, Human mastadenovirus A
MSKDIPTPYMWSFQPQMGLAAGAAQDYSSK

SEQ ID NO: 136 - protein, 30 aa, Human mastadenovirus A
PPGFYTGEFDLPEGNDGFLWDDVTDSLFSP

SEQ ID NO: 137 - protein, 30 aa, Human mastadenovirus A
VLEYMKVDPNIQPDVKIRPIKKVAPGLGVQ

SEQ ID NO: 138 - protein, 30 aa, Human mastadenovirus A
EAPPPSYETVMAAAQTSALEAPYVPPRYLA

SEQ ID NO: 139 - protein, 30 aa, Human mastadenovirus A
GHYRAPWGAHTRGRTGRTTVDDVIDSVVAD

SEQ ID NO: 140 - protein, 30 aa, Human mastadenovirus A
LKDQNFQQKVVDGLASGINGVVDIANQAVQ

SEQ ID NO: 141 - protein, 30 aa, Human mastadenovirus A
NNPQVVFYTEDVNLEMPDTHLVFKPTVTDG

SEQ ID NO: 142 - protein, 30 aa, Human mastadenovirus B
RASRRRQRHDRQRGLVWEDEDSADDSSVLD

SEQ ID NO: 143 - protein, 30 aa, Human mastadenovirus B
WPAALVYQESPAPTTVLLPRDAQAEVQMTN

SEQ ID NO: 144 - protein, 30 aa, Human mastadenovirus B
TLVTRADEPPSYEEAVKLGMPTTRPVAHMA

SEQ ID NO: 145 - protein, 30 aa, Human mastadenovirus B
SFNPVYPYEDESTSQHPFINPGFISPNGFT

SEQ ID NO: 146 - protein, 30 aa, Human mastadenovirus B
TVDDTDGTLQENIGTTTPLVKTGHSIGLSL

SEQ ID NO: 147 - protein, 30 aa, Human mastadenovirus B
PPLQPFDPPTLHDLYDLEVDGPDDPNEEAV

SEQ ID NO: 148 - protein, 30 aa, Human mastadenovirus B
EEIEADVEQDPGYVTPAEHEEELKRFLDRE

SEQ ID NO: 149 - protein, 30 aa, Human mastadenovirus C
DMNDHAIRGDTFATRAEEKRAEAEAAAEAA

SEQ ID NO: 150 - protein, 30 aa, Human mastadenovirus C
AATQKQRRPDSKTLTKPKKSTAAAAAGGGA

SEQ ID NO: 151 - protein, 30 aa, Human mastadenovirus C
TTDYRNTTATGLTSALNLPQVHAFVNDWAS

SEQ ID NO: 152 - protein, 30 aa, Human mastadenovirus C
PSAPAVSTVDEAIESVVQGARHYANLKNRR

SEQ ID NO: 153 - protein, 30 aa, Human mastadenovirus C
ARNYTPTPPPVSTVDAAIQTVVRGARRYAK

SEQ ID NO: 154 - protein, 30 aa, Human mastadenovirus C
EVLDEEEEMMEDWDSLDEEASEAEEVSDET

SEQ ID NO: 155 - protein, 30 aa, Human mastadenovirus C
ARRTGRRAAMRAARRLAAGIVTVPPRSRRR

SEQ ID NO: 156 - protein, 30 aa, Human mastadenovirus C
IAPMATGVLGHHTPVTLDLPPPADTQQKPV

SEQ ID NO: 157 - protein, 30 aa, Human mastadenovirus C
SYESVVSAASVAAALGSPFDAPLDPPFVPP

SEQ ID NO: 158 - protein, 30 aa, Human mastadenovirus C
TTRPRLLGEEEYLNNSLLQPQREKNLPPAF

SEQ ID NO: 159 - protein, 30 aa, Human mastadenovirus D
PEDARPVVSDEMLARWLGTRDPQALEQRRK

SEQ ID NO: 160 - protein, 30 aa, Human mastadenovirus D
MTKRLRVEDDFNPIYPYGYARNQNIPFLTP

SEQ ID NO: 161 - protein, 30 aa, Human mastadenovirus D
ESYKNEIKKLTYKNNKTTFEDSGNYEHQKL

SEQ ID NO: 162 - protein, 30 aa, Human orthopneumovirus
EKINQSLTFIRKSDELLHNVNVGKSTTNIM

SEQ ID NO: 163 - protein, 30 aa, Human orthopneumovirus
FEAFNFVPCSICSGNPTCWAICKRIPNKKP

SEQ ID NO: 164 - protein, 30 aa, Human orthopneumovirus
NYQRKPLVSFKEDPTPSDNPFSKLYKETIE

SEQ ID NO: 165 - protein, 30 aa, Human orthopneumovirus
NKLGLKEKEKDKIKSNNEQDENNSVITTII

SEQ ID NO: 166 - protein, 30 aa, Human orthopneumovirus
STYMLTNSELLSLINDMPITNDQKKLMSSN

SEQ ID NO: 167 - protein, 30 aa, Human orthopneumovirus
TNKPSTKPHPKIPPKKPKDDYHFEVFNFVP

SEQ ID NO: 168 - protein, 30 aa, Human orthopneumovirus
CSICGNNQLCKSICKTIPGNKPKKKPTIKP

SEQ ID NO: 169 - protein, 30 aa, Human papillomavirus
QAITCQKPTPEKEKPDPYKNLSFWEVNLKE

SEQ ID NO: 170 - protein, 30 aa, Human papillomavirus
PPAEKKDPYADLTFWEVDLKERFSLELDQF

SEQ ID NO: 171 - protein, 30 aa, Human papillomavirus
AGKEGDSIPMEGTDYYIARQDSKLASHIYY

SEQ ID NO: 172 - protein, 30 aa, Human polyomavirus 1
AHQPDFGTWNSSEVPTYGTEEWESWWSSFN

SEQ ID NO: 173 - protein, 30 aa, Human polyomavirus 1
RVFDGTERLPGDPDMIRYIDKQGQLQTKML

SEQ ID NO: 174 - protein, 30 aa, Human polyomavirus 3
LSDEIQRLLRDLEYGFRATLASIGESDPVN

SEQ ID NO: 175 - protein, 30 aa, Human polyomavirus 3
NIWQSSQIPTYGTPDWDEWWSQFNTYWEEE

SEQ ID NO: 176 - protein, 30 aa, Human polyomavirus 3
LQSVHKPIHAPYSGMALVPIPEYQLETGIP

SEQ ID NO: 177 - protein, 30 aa, Human respirovirus 1
DEDITDIENKIARRLADRKQRLSQANNKRD

SEQ ID NO: 178 - protein, 30 aa, Human respirovirus 1
NETTDYSSEGIEDLVFDILDLKGKTKSHRY

SEQ ID NO: 179 - protein, 30 aa, Human respirovirus 1
GAIEVAIDHTDITFGAEDTADRDNKNWAND

SEQ ID NO: 180 - protein, 30 aa, Human respirovirus 3
KKQGSQPPTNPTNRTNQDEIDDLFNAFGSN

SEQ ID NO: 181 - protein, 30 aa, Human respirovirus 3
EQATESDNIKTEQQNIRDRLNKRLNDKKKQ

SEQ ID NO: 182 - protein, 30 aa, Human respirovirus 3
IVLINSIKSERAHESLLQDINNEFMEVTEK

SEQ ID NO: 183 - protein, 30 aa, Human respirovirus 3
ENRADQEQGGEPQSSIIQYAWAEGNRNDDR

SEQ ID NO: 184 - protein, 30 aa, Human respirovirus 3
SIKSEKAHESLLRDINNEFIGITEKIQMAS

SEQ ID NO: 185 - protein, 30 aa, Human respirovirus 3
STHQEDDKRIKKGGKGKDWFKKSKDTDNQT

SEQ ID NO: 186 - protein, 30 aa, Human respirovirus 3
MESDAKNYQVMDSWEEESRDKSTNISSALN

SEQ ID NO: 187 - protein, 30 aa, Human respirovirus 3
SLESIGTPDTRSISVVTAATPDDEEEILMK

SEQ ID NO: 188 - protein, 30 aa, Human rubulavirus 2
PQRTSGMSSEEFQHSMNQYIRAMHEQYRGS

SEQ ID NO: 189 - protein, 30 aa, Human rubulavirus 2
MAEEPTYTTEQVDELIHAGLGTVDFFLSRP

SEQ ID NO: 190 - protein, 30 aa, Human rubulavirus 2
QLPRGRQPISDPFAGANDREIGGQANDTPV

SEQ ID NO: 191 - protein, 30 aa, Influenza A virus
PSSSAGLKDDLLENLQAYQKRMGVQMQRFK

SEQ ID NO: 192 - protein, 30 aa, Influenza A virus
LATGMRNVPEKQTRGIFGAIAGFIENGWEG

SEQ ID NO: 193 - protein, 30 aa, Influenza A virus
SEQAAEAMEIASQARQMVQAMRTVGTHPSS

SEQ ID NO: 194 - protein, 30 aa, Influenza A virus
NPLIRHENRMVLASTTAKAMEQMAGSSEQA

SEQ ID NO: 195 - protein, 30 aa, Influenza A virus
RTLDYHDSNVKNLYEKVRSQLKNNAKEIGN

SEQ ID NO: 196 - protein, 30 aa, Influenza A virus
IWDPNGWTGTDNNFSIKQDIVGINEWSGYS

SEQ ID NO: 197 - protein, 30 aa, Influenza A virus
ATCEQIADAQHRSHRQMATTTNPLIKHENR

SEQ ID NO: 198 - protein, 30 aa, Influenza A virus
VETYVLSIIPSGPLKAEIAQKLEDVFAGKN

SEQ ID NO: 199 - protein, 30 aa, Influenza A virus
NPHRILDGIDCTLIDALLGDPHCDGFQNET

SEQ ID NO: 200 - protein, 30 aa, Influenza B virus
FAVERPIALSKQAVRKMLSMNIEGRDADVK

SEQ ID NO: 201 - protein, 30 aa, Influenza B virus
TTRPIIRPATLAPPSNKRTRNPSPERATTS

SEQ ID NO: 202 - protein, 30 aa, Influenza B virus
GTFNAGEFSLPTFDSLNITAASLNNDGLDN

SEQ ID NO: 203 - protein, 30 aa, Influenza B virus
RTRGKLCPECLNCTDLDVALGRPMCVGTTP

SEQ ID NO: 204 - protein, 30 aa, Influenza B virus
TNPIEIPIKQTIPNFFFGRDTAEDYDDLDY

SEQ ID NO: 205 - protein, 30 aa, Influenza B virus
SLSELEVKNLQRLSGAMDELHSEILELDEK

SEQ ID NO: 206 - protein, 30 aa, Influenza B virus
SNSPHVVKTATQGEVNVTGVIPLTTTPTKS

SEQ ID NO: 207 - protein, 30 aa, Influenza B virus
MSNMDIDGINTGTIDKAPEEITSGTSGTTR

SEQ ID NO: 208 - protein, 30 aa, Influenza B virus
NTAKTMNGMGKGEDVQKLAEELQSNIGVLR

SEQ ID NO: 209 - protein, 30 aa, Influenza C virus
GEADDHHGDQEMRELLSGLDYEARCISQSG

SEQ ID NO: 210 - protein, 30 aa, Influenza C virus
YLLPPKFGRCPLAAKEESIPKIPDGLLIPT

SEQ ID NO: 211 - protein, 30 aa, Macaca mulatta polyomavirus 1
PTYGTDEWEQWWNAFNEENLFCSEEMPSSD

SEQ ID NO: 212 - protein, 30 aa, Macaca mulatta polyomavirus 1
VGVLDWLRNSDDDDDDEDGGEKNMEDSGHE

SEQ ID NO: 213 - protein, 30 aa, Mamastrovirus 1
RELVINTLVNQGISRDRATYIGMSAYPNVE

SEQ ID NO: 214 - protein, 30 aa, Mamastrovirus 1
DIIDTSDEEYENETDRVTLLSTLVNQGMTM

SEQ ID NO: 215 - protein, 30 aa, Mamastrovirus 1
FDEDPIATLHAVDAERKIRRAIFNALMEEG

SEQ ID NO: 216 - protein, 30 aa, Mamastrovirus 1
DQREKYRHVHEWYVDNLLNRHVLLPSGEVT

SEQ ID NO: 217 - protein, 30 aa, Mamastrovirus 1
TSIPRSRASGHGYESDNTEYLDAPDSADQF

SEQ ID NO: 218 - protein, 30 aa, Mamastrovirus 1
EEYGPTPWGPQAFIKSFDKFFYAEPIDFFS

SEQ ID NO: 219 - protein, 30 aa, Mamastrovirus 1
DRATLLSTLLNQGISVERATRITNGAFPAR

SEQ ID NO: 220 - protein, 30 aa, Mamastrovirus 1
DDEADRFDLHSSYGSEPEDDDENNRVTLLS

SEQ ID NO: 221 - protein, 30 aa, Mamastrovirus 6
VTSDDTDYDTDTEDEDEFFGEDPIAALHAV

SEQ ID NO: 222 - protein, 30 aa, Mamastrovirus 6
SADGANEPVEMLIPVNEWNMKAQYGGNGTL

SEQ ID NO: 223 - protein, 30 aa, Norwalk virus
KGLSDEEYEEYKRVREERNGKYSIEEYLQD

SEQ ID NO: 224 - protein, 30 aa, Norwalk virus
DDFKLKGKLWADDDRSVDYNERLNFEAPPS

SEQ ID NO: 225 - protein, 30 aa, Norwalk virus
ISGLPDLTTVPQPDATNTAFSVPPLSLREN

SEQ ID NO: 226 - protein, 30 aa, Norwalk virus
APDIEKAKRDFPGQPDMWKDHFRPDFSHIK

SEQ ID NO: 227 - protein, 30 aa, Norwalk virus
TTGFFRPYQDWNKKPLPTVDDSKLKKVANI

SEQ ID NO: 228 - protein, 30 aa, Norwalk virus
NNYDPTEEIPAPLGTPDFVGKIQGVLTQTT

SEQ ID NO: 229 - protein, 30 aa, Norwalk virus
AYSVPPLSQREVGEAKEPLPGSILEMWDGE

SEQ ID NO: 230 - protein, 30 aa, Norwalk virus
DEEYDEYKKIREERGGKYSIQEYLEDRERF

SEQ ID NO: 231 - protein, 30 aa, Norwalk virus
MMMASKDAPTNMDGTSGAGQLVPEANTAEP

SEQ ID NO: 232 - protein, 30 aa, Norwalk virus
VVSYSVKDGVSGLPDLSTVRQPEESNTAFS

SEQ ID NO: 233 - protein, 30 aa, Parechovirus A
QDIHLIDDLGQTRKEKDIEMLCNCISSVPF

SEQ ID NO: 234 - protein, 30 aa, Parechovirus A
TTNLTQHPSAPTIPFTPDFRNVDNFHSMAY

SEQ ID NO: 235 - protein, 30 aa, Parechovirus A
SAPTMPFTPDFSNVDTFHSMAYDVTTGEKN

SEQ ID NO: 236 - protein, 30 aa, Parechovirus A
QIHKSPVYGAVEVKMGPAVLSKSDPRLEEP

SEQ ID NO: 237 - protein, 30 aa, Rhinovirus A
VPITQNPVENYIDEVLNEVLVVPNIKESHP

SEQ ID NO: 238 - protein, 30 aa, Rhinovirus A
EQYIDGVLNEVLIVPNINESHPSTSNAAPA

SEQ ID NO: 239 - protein, 30 aa, Rhinovirus A
SAIFQGPISLGAPPPPAIADLLQSVRTPEV

SEQ ID NO: 240 - protein, 30 aa, Rhinovirus A
IFQGPIDMRNPPPPAITDLLQAVRTPEVIK

SEQ ID NO: 241 - protein, 30 aa, Rhinovirus A
ADEQGITDYIHTLGEAFGAGFVDNIKDQIQ

SEQ ID NO: 242 - protein, 30 aa, Rhinovirus A
VMEQNPVEKYTEAVLNEVLAVPNITPSNSQ

SEQ ID NO: 243 - protein, 30 aa, Rhinovirus A
NPSGEDMTLFCQMVSSVPFIPPMADLPDKG

SEQ ID NO: 244 - protein, 30 aa, Rhinovirus A
KLQPSVFYDVFPGSKEPAVLTSNDPRLEVD

SEQ ID NO: 245 - protein, 30 aa, Rhinovirus A
DFIADEQGLGDYITSLGRAFGTGFTDQISA

SEQ ID NO: 246 - protein, 30 aa, Rhinovirus A
PPPPAIMDLLKSVKNPEVIKYCEDNKWIIP

SEQ ID NO: 247 - protein, 30 aa, Rhinovirus A
TISQTDALTEGLGDELEEVIVEKTKQTLAS

SEQ ID NO: 248 - protein, 30 aa, Rhinovirus B
ECINDLLRSVDSEEVREYCKRKNWIIPQIP

SEQ ID NO: 249 - protein, 30 aa, Rhinovirus B
QSLHQETALTEGLEDELMEVIVDKTQQTLA

SEQ ID NO: 250 - protein, 30 aa, Rhinovirus B
VVPEHQLASHTQGNVSVKYKYTHPGEQGID

SEQ ID NO: 251 - protein, 30 aa, Rhinovirus B
QLASHDGGTVSVKYKFTHPGDQGIDLSTAE

SEQ ID NO: 252 - protein, 30 aa, Rhinovirus B
ISDLLKSVDSEEIREYCKQKNWLIPEIPTN

SEQ ID NO: 253 - protein, 30 aa, Rhinovirus B
HTQSVPALTANETGATLPTRPSDNVETRTT

SEQ ID NO: 254 - protein, 30 aa, Rhinovirus B
AATKMDFSQDPSKFTEPVKDVMIKTAPALN

SEQ ID NO: 255 - protein, 30 aa, Rhinovirus B
EDELEEVVIDKMKQVTASSQSGPKHTQKVP

SEQ ID NO: 256 - protein, 30 aa, Rhinovirus B
ELNMNPINTPTKSKLHPSVFYNVFPGDKEP

SEQ ID NO: 257 - protein, 30 aa, Rhinovirus B
LMKDTQTISQTEALTEGFEEELEEVVVDKM

SEQ ID NO: 258 - protein, 30 aa, Rhinovirus B
DVLEEVIVDKAKQTIASINSNSKYTQQVPT

SEQ ID NO: 259 - protein, 30 aa, Rhinovirus C
VEHNLTAIFQGLGDDTTPGFIIDLLSASKD

SEQ ID NO: 260 - protein, 30 aa, Rhinovirus C
NPVEDFIDTTLKEVLVVPDTHPSGPVHTTR

SEQ ID NO: 261 - protein, 30 aa, Rhinovirus C
AHQGLVSDYVNQLGAAFGDGFSSNIKDHLT

SEQ ID NO: 262 - protein, 30 aa, Rhinovirus C
AYIGGTNANVGYNHTHPGEIGHEIGRNTGR

SEQ ID NO: 263 - protein, 30 aa, Rhinovirus C
TNDLQNNDPIDTYVHDVLNEVVVVPDTKPS

SEQ ID NO: 264 - protein, 30 aa, Rhinovirus C
DTPMITQDKNTLQNPVEQFVDDVLEEVLVV

SEQ ID NO: 265 - protein, 30 aa, Rhinovirus C
IPEHQLAYAGGANASVGYKHTHPGENGHKI

SEQ ID NO: 266 - protein, 30 aa, Rhinovirus C
SSLSEHQGVTDYITQLGSAFGDGFTSSIKQ

SEQ ID NO: 267 - protein, 30 aa, Rhinovirus C
LRPYNNLAQTQGPISDYVTQLGNAFGNGFT

SEQ ID NO: 268 - protein, 30 aa, Rhinovirus C
DPVSDFIDATLQEVLVVPETKPSGPQHTTK

SEQ ID NO: 269 - protein, 30 aa, Rhinovirus C
PINTPSTTKLYPSVFYEIFPGEKEPAVLSD

SEQ ID NO: 270 - protein, 30 aa, Rotavirus A
IDMSKEFNQKNIKTLDEWESGKNPYEPSEV

SEQ ID NO: 271 - protein, 30 aa, Rotavirus A
NERLQEKEIEKNADAIMENKNGNKKQQLSD

SEQ ID NO: 272 - protein, 30 aa, Rotavirus A
KEIENNTDVTMENKNKNKNKNNNRKQQLSD

SEQ ID NO: 273 - protein, 30 aa, Rotavirus C
KRRNVQQKDVEKEKQIEKMEEKEIKEVKEQ

SEQ ID NO: 274 - protein, 30 aa, Rotavirus H
IKTVPLENELKQKEKQRDNKEKNEKENKDE

SEQ ID NO: 275 - protein, 30 aa, Severe acute respiratory syndrome coronavirus 2
FNGLTGTGVLTESNKKFLPFQQFGRDIADT

SEQ ID NO: 276 - protein, 30 aa, Severe acute respiratory syndrome coronavirus 2
GVLTESNKKFLPFQQFGRDIADTTDAVRDP

SEQ ID NO: 277 - protein, 30 aa, Severe acute respiratory syndrome coronavirus 2
KKFLPFQQFGRDIADTTDAVRDPQTLEILD

SEQ ID NO: 278 - protein, 30 aa, Severe acute respiratory syndrome coronavirus 2
KFLPFQQFGRDIADTTDAVRDPQTLEILDI

SEQ ID NO: 279 - protein, 30 aa, Severe acute respiratory syndrome coronavirus 2
FLPFQQFGRDIADTTDAVRDPQTLEILDIT

SEQ ID NO: 280 - protein, 30 aa, Severe acute respiratory syndrome coronavirus 2
LPFQQFGRDIADTTDAVRDPQTLEILDITP

SEQ ID NO: 281 - protein, 30 aa, Severe acute respiratory syndrome coronavirus 2
QILPDPSKPSKRSFIEDLLFNKVTLADAGF

SEQ ID NO: 282 - protein, 30 aa, Severe acute respiratory syndrome coronavirus 2
DPSKPSKRSFIEDLLFNKVTLADAGFIKQY

SEQ ID NO: 283 - protein, 30 aa, Severe acute respiratory syndrome coronavirus 2
PSKPSKRSFIEDLLFNKVTLADAGFIKQYG

SEQ ID NO: 284 - protein, 30 aa, Severe acute respiratory syndrome coronavirus 2
SSVLNDILSRLDKVEAEVQIDRLITGRLQS

SEQ ID NO: 285 - protein, 30 aa, Severe acute respiratory syndrome coronavirus 2
NTVYDPLQPELDSFKEELDKYFKNHTSPDV

SEQ ID NO: 286 - protein, 30 aa, Severe acute respiratory syndrome coronavirus 2
TVYDPLQPELDSFKEELDKYFKNHTSPDVD

SEQ ID NO: 287 - protein, 30 aa, Severe acute respiratory syndrome coronavirus 2
YDPLQPELDSFKEELDKYFKNHTSPDVDLG

SEQ ID NO: 288 - protein, 30 aa, Severe acute respiratory syndrome coronavirus 2
DPLQPELDSFKEELDKYFKNHTSPDVDLGD

SEQ ID NO: 289 - protein, 30 aa, Severe acute respiratory syndrome coronavirus 2
LQPELDSFKEELDKYFKNHTSPDVDLGDIS

SEQ ID NO: 290 - protein, 30 aa, Severe acute respiratory syndrome coronavirus 2
QPELDSFKEELDKYFKNHTSPDVDLGDISG

SEQ ID NO: 291 - protein, 30 aa, Severe acute respiratory syndrome coronavirus 2
PELDSFKEELDKYFKNHTSPDVDLGDISGI

SEQ ID NO: 292 - protein, 30 aa, Severe acute respiratory syndrome coronavirus 2
ELDSFKEELDKYFKNHTSPDVDLGDISGIN

SEQ ID NO: 293 - protein, 30 aa, Severe acute respiratory syndrome coronavirus 2
LDSFKEELDKYFKNHTSPDVDLGDISGINA

SEQ ID NO: 294 - protein, 30 aa, Severe acute respiratory syndrome coronavirus 2
DSFKEELDKYFKNHTSPDVDLGDISGINAS

SEQ ID NO: 295 - protein, 30 aa, Severe acute respiratory syndrome coronavirus 2
NTPKDHIGTRNPANNAAIVLQLPQGTTLPK

SEQ ID NO: 296 - protein, 30 aa, Severe acute respiratory syndrome coronavirus 2
TPKDHIGTRNPANNAAIVLQLPQGTTLPKG

SEQ ID NO: 297 - protein, 30 aa, Severe acute respiratory syndrome coronavirus 2
PKDHIGTRNPANNAAIVLQLPQGTTLPKGF

SEQ ID NO: 298 - protein, 30 aa, Severe acute respiratory syndrome coronavirus 2
KDHIGTRNPANNAAIVLQLPQGTTLPKGFY

SEQ ID NO: 299 - protein, 30 aa, Severe acute respiratory syndrome coronavirus 2
DHIGTRNPANNAAIVLQLPQGTTLPKGFYA

SEQ ID NO: 300 - protein, 30 aa, Severe acute respiratory syndrome coronavirus 2
HIGTRNPANNAAIVLQLPQGTTLPKGFYAE

SEQ ID NO: 301 - protein, 30 aa, Severe acute respiratory syndrome coronavirus 2
IGTRNPANNAAIVLQLPQGTTLPKGFYAEG

SEQ ID NO: 302 - protein, 30 aa, Severe acute respiratory syndrome coronavirus 2
GTRNPANNAAIVLQLPQGTTLPKGFYAEGS

SEQ ID NO: 303 - protein, 30 aa, Severe acute respiratory syndrome coronavirus 2
TRNPANNAAIVLQLPQGTTLPKGFYAEGSR

SEQ ID NO: 304 - protein, 30 aa, Severe acute respiratory syndrome coronavirus 2
RNPANNAAIVLQLPQGTTLPKGFYAEGSRG

SEQ ID NO: 305 - protein, 30 aa, Severe acute respiratory syndrome coronavirus 2
NPANNAAIVLQLPQGTTLPKGFYAEGSRGG

SEQ ID NO: 306 - protein, 30 aa, Severe acute respiratory syndrome coronavirus 2
PANNAAIVLQLPQGTTLPKGFYAEGSRGGS

SEQ ID NO: 307 - protein, 30 aa, Severe acute respiratory syndrome coronavirus 2
ANNAAIVLQLPQGTTLPKGFYAEGSRGGSQ

SEQ ID NO: 308 - protein, 30 aa, Severe acute respiratory syndrome coronavirus 2
NNAAIVLQLPQGTTLPKGFYAEGSRGGSQA

SEQ ID NO: 309 - protein, 30 aa, Severe acute respiratory syndrome coronavirus 2
AAIVLQLPQGTTLPKGFYAEGSRGGSQASS

SEQ ID NO: 310 - protein, 30 aa, Severe acute respiratory syndrome coronavirus 2
AIVLQLPQGTTLPKGFYAEGSRGGSQASSR

SEQ ID NO: 311 - protein, 30 aa, Severe acute respiratory syndrome coronavirus 2
IVLQLPQGTTLPKGFYAEGSRGGSQASSRS

SEQ ID NO: 312 - protein, 30 aa, Severe acute respiratory syndrome coronavirus 2
VLQLPQGTTLPKGFYAEGSRGGSQASSRSS

SEQ ID NO: 313 - protein, 30 aa, Severe acute respiratory syndrome coronavirus 2
LQLPQGTTLPKGFYAEGSRGGSQASSRSSS

SEQ ID NO: 314 - protein, 30 aa, Severe acute respiratory syndrome coronavirus 2
QLPQGTTLPKGFYAEGSRGGSQASSRSSSR

SEQ ID NO: 315 - protein, 30 aa, Severe acute respiratory syndrome coronavirus 2
LPQGTTLPKGFYAEGSRGGSQASSRSSSRS

SEQ ID NO: 316 - protein, 30 aa, Severe acute respiratory syndrome coronavirus 2
PQGTTLPKGFYAEGSRGGSQASSRSSSRSR

SEQ ID NO: 317 - protein, 30 aa, Severe acute respiratory syndrome coronavirus 2
QGTTLPKGFYAEGSRGGSQASSRSSSRSRN

SEQ ID NO: 318 - protein, 30 aa, Severe acute respiratory syndrome coronavirus 2
AGNGGDAALALLLLDRLNQLESKMSGKGQQ

SEQ ID NO: 319 - protein, 30 aa, Severe acute respiratory syndrome coronavirus 2
IGTRNPANNASIVLQLPQGTTLPKGFYAEG

SEQ ID NO: 320 - protein, 30 aa, Severe acute respiratory syndrome coronavirus 2
GTRNPSNNAAIVLQLPQGTTLPKGFYAEGS

SEQ ID NO: 321 - protein, 30 aa, Severe acute respiratory syndrome coronavirus 2
QLPQGTTLPKGFYAEGSRGGSQASSRSSLR

SEQ ID NO: 322 - protein, 30 aa, Severe acute respiratory syndrome coronavirus 2
AIVLQLPQGTTLPKGFYAEGSRGGSQAYSR

SEQ ID NO: 323 - protein, 30 aa, Severe acute respiratory syndrome coronavirus 2
IGTRNPANNAAIVLQLPQGTTLPKGFYAKG

SEQ ID NO: 324 - protein, 30 aa, Severe acute respiratory syndrome coronavirus 2
GIVNNTVYDPLQPELESFKEELDKYFKNHT

SEQ ID NO: 325 - protein, 30 aa, Severe acute respiratory syndrome coronavirus 2
VNNTVYDPLQPELESFKEELDKYFKNHTSP

SEQ ID NO: 326 - protein, 30 aa, Severe acute respiratory syndrome coronavirus 2
YDPLQPELDSFKEELDKYFKNHTSLDVDLG

SEQ ID NO: 327 - protein, 30 aa, Severe acute respiratory syndrome coronavirus 2
TGVLTESNKKFLPFQQFGRDIADTTDAVRD

SEQ ID NO: 328 - protein, 30 aa, Severe acute respiratory syndrome coronavirus 2
DFGGFNFSQILPDPSKPSKRSFIEDLLFNK

SEQ ID NO: 329 - protein, 30 aa, Severe acute respiratory syndrome coronavirus 2
GGFNFSQILPDPSKPSKRSFIEDLLFNKVT

SEQ ID NO: 330 - protein, 30 aa, Severe acute respiratory syndrome coronavirus 2
NFSQILPDPSKPSKRSFIEDLLFNKVTLAD

SEQ ID NO: 331 - protein, 30 aa, Severe acute respiratory syndrome coronavirus 2
SQILPDPSKPSKRSFIEDLLFNKVTLADAG

SEQ ID NO: 332 - protein, 30 aa, Severe acute respiratory syndrome coronavirus 2
ILPDPSKPSKRSFIEDLLFNKVTLADAGFI

SEQ ID NO: 333 - protein, 30 aa, Severe acute respiratory syndrome coronavirus 2
LPDPSKPSKRSFIEDLLFNKVTLADAGFIK

SEQ ID NO: 334 - protein, 30 aa, Severe acute respiratory syndrome coronavirus 2
PDPSKPSKRSFIEDLLFNKVTLADAGFIKQ

SEQ ID NO: 335 - protein, 30 aa, Severe acute respiratory syndrome coronavirus 2
KPSKRSFIEDLLFNKVTLADAGFIKQYGDC

SEQ ID NO: 336 - protein, 30 aa, Severe acute respiratory syndrome coronavirus 2
PSKRSFIEDLLFNKVTLADAGFIKQYGDCL

SEQ ID NO: 337 - protein, 30 aa, Severe acute respiratory syndrome coronavirus 2
SKRSFIEDLLFNKVTLADAGFIKQYGDCLG

SEQ ID NO: 338 - protein, 30 aa, Severe acute respiratory syndrome coronavirus 2
KRSFIEDLLFNKVTLADAGFIKQYGDCLGD

SEQ ID NO: 339 - protein, 30 aa, Severe acute respiratory syndrome coronavirus 2
FIEDLLFNKVTLADAGFIKQYGDCLGDIAA

SEQ ID NO: 340 - protein, 30 aa, Severe acute respiratory syndrome coronavirus 2
IEDLLFNKVTLADAGFIKQYGDCLGDIAAR

SEQ ID NO: 341 - protein, 30 aa, Severe acute respiratory syndrome coronavirus 2
EDLLFNKVTLADAGFIKQYGDCLGDIAARD

SEQ ID NO: 342 - protein, 30 aa, Severe acute respiratory syndrome coronavirus 2
YFKNHTSPDVDLGDIPGINASVVNIQKEID

SEQ ID NO: 343 - protein, 30 aa, Severe acute respiratory syndrome coronavirus 2
LPDPSKPSKRSFIEDILFNKVTLADAGFIK

SEQ ID NO: 344 - protein, 30 aa, Severe acute respiratory syndrome coronavirus 2
FSQILPDSSKPSKRSFIEDLLFNKVTLADA

SEQ ID NO: 345 - protein, 30 aa, Severe acute respiratory syndrome coronavirus 2
FKNLREFVFKNIDGYFNIYSKHTPINLVRD

SEQ ID NO: 346 - protein, 30 aa, Severe acute respiratory syndrome coronavirus 2
KDFGGFNFSQILPDPSKPSKRSFIEDLLFN

SEQ ID NO: 347 - protein, 30 aa, Severe acute respiratory syndrome coronavirus 2
GFNFSQILPDPSKPSKRSFIEDLLFNKVTL

SEQ ID NO: 348 - protein, 30 aa, Severe acute respiratory syndrome coronavirus 2
DVVIGIVNNTVYDPLQPELDSFKEELDKYF

SEQ ID NO: 349 - protein, 30 aa, Severe acute respiratory syndrome coronavirus 2
VVIGIVNNTVYDPLQPELDSFKEELDKYFK

SEQ ID NO: 350 - protein, 30 aa, Severe acute respiratory syndrome coronavirus 2
VIGIVNNTVYDPLQPELDSFKEELDKYFKN

SEQ ID NO: 351 - protein, 30 aa, Severe acute respiratory syndrome coronavirus 2
IGIVNNTVYDPLQPELDSFKEELDKYFKNH

SEQ ID NO: 352 - protein, 30 aa, Severe acute respiratory syndrome coronavirus 2
GIVNNTVYDPLQPELDSFKEELDKYFKNHT

SEQ ID NO: 353 - protein, 30 aa, Severe acute respiratory syndrome coronavirus 2
IVNNTVYDPLQPELDSFKEELDKYFKNHTS

SEQ ID NO: 354 - protein, 30 aa, Severe acute respiratory syndrome coronavirus 2
VNNTVYDPLQPELDSFKEELDKYFKNHTSP

SEQ ID NO: 355 - protein, 30 aa, Severe acute respiratory syndrome coronavirus 2
NNTVYDPLQPELDSFKEELDKYFKNHTSPD

SEQ ID NO: 356 - protein, 30 aa, Severe acute respiratory syndrome coronavirus 2
VYDPLQPELDSFKEELDKYFKNHTSPDVDL

SEQ ID NO: 357 - protein, 30 aa, Severe acute respiratory syndrome coronavirus 2
PLQPELDSFKEELDKYFKNHTSPDVDLGDI

SEQ ID NO: 358 - protein, 30 aa, Severe acute respiratory syndrome coronavirus 2
FKEELDKYFKNHTSPDVDLGDISGINASVV

SEQ ID NO: 359 - protein, 30 aa, Severe acute respiratory syndrome coronavirus 2
LQLELDSFKEELDKYFKNHTSPDVDLGDIS

SEQ ID NO: 360 - protein, 30 aa, Severe acute respiratory syndrome coronavirus 2
FKEELDKYFKNHTSLDVDLGDISGINASVV

SEQ ID NO: 361 - protein, 30 aa, Severe acute respiratory syndrome coronavirus 2
NNAAIVLQLPQGTTLSKGFYAEGSRGGSQA

SEQ ID NO: 362 - protein, 30 aa, Severe acute respiratory syndrome coronavirus 2
DRLNEVAKNLNESLIDLQELGKYEQYIKWP

SEQ ID NO: 363 - protein, 30 aa, Severe acute respiratory syndrome coronavirus 2
LNEVAKNLNESLIDLQELGKYEQYIKWPWY

SEQ ID NO: 364 - protein, 30 aa, Severe acute respiratory syndrome coronavirus 2
KLDDKDPNFKDQVILLNKHIDAYKTFPPTE

SEQ ID NO: 365 - protein, 30 aa, Severe acute respiratory syndrome coronavirus 2
GAISSVLNDILSRLDKVEAEVQIDRLITGR

SEQ ID NO: 366 - protein, 30 aa, Severe acute respiratory syndrome coronavirus 2
ISSVLNDILSRLDKVEAEVQIDRLITGRLQ

SEQ ID NO: 367 - protein, 30 aa, Severe acute respiratory syndrome coronavirus 2
VLNDILSRLDKVEAEVQIDRLITGRLQSLQ

SEQ ID NO: 368 - protein, 30 aa, Severe acute respiratory syndrome coronavirus 2
LNDILSRLDKVEAEVQIDRLITGRLQSLQT

SEQ ID NO: 369 - protein, 30 aa, Severe acute respiratory syndrome coronavirus 2
DETQALPQRQKKQQIVTLLPAADLDDFSKQ

SEQ ID NO: 370 - protein, 30 aa, Severe acute respiratory syndrome coronavirus 2
AVRDPQTLEILDITPCSFGGVSVITPGTNT

SEQ ID NO: 371 - protein, 30 aa, Severe acute respiratory syndrome coronavirus 2
RATRRIRGGDGKMKDLSPRWYFYYLGTGPE

SEQ ID NO: 372 - protein, 30 aa, Severe acute respiratory syndrome coronavirus 2
ATRRIRGGDGKMKDLSPRWYFYYLGTGPEA

SEQ ID NO: 373 - protein, 30 aa, Severe acute respiratory syndrome coronavirus 2
RIRGGDGKMKDLSPRWYFYYLGTGPEAGLP

SEQ ID NO: 374 - protein, 30 aa, Severe acute respiratory syndrome coronavirus 2
IRGGDGKMKDLSPRWYFYYLGTGPEAGLPY

SEQ ID NO: 375 - protein, 30 aa, Severe acute respiratory syndrome coronavirus 2
RGGDGKMKDLSPRWYFYYLGTGPEAGLPYG

SEQ ID NO: 376 - protein, 30 aa, Severe acute respiratory syndrome coronavirus 2
GGDGKMKDLSPRWYFYYLGTGPEAGLPYGA

SEQ ID NO: 377 - protein, 30 aa, Severe acute respiratory syndrome coronavirus 2
GDGKMKDLSPRWYFYYLGTGPEAGLPYGAN

SEQ ID NO: 378 - protein, 30 aa, Severe acute respiratory syndrome coronavirus 2
DGKMKDLSPRWYFYYLGTGPEAGLPYGANK

SEQ ID NO: 379 - protein, 30 aa, Severe acute respiratory syndrome coronavirus 2
GKMKDLSPRWYFYYLGTGPEAGLPYGANKD

SEQ ID NO: 380 - protein, 30 aa, Severe acute respiratory syndrome coronavirus 2
KMKDLSPRWYFYYLGTGPEAGLPYGANKDG

SEQ ID NO: 381 - protein, 30 aa, Severe acute respiratory syndrome coronavirus 2
MKDLSPRWYFYYLGTGPEAGLPYGANKDGI

SEQ ID NO: 382 - protein, 30 aa, Severe acute respiratory syndrome coronavirus 2
GTTLPKGFYAEGSRGGSQASSRSSSRSRNS

SEQ ID NO: 383 - protein, 30 aa, Severe acute respiratory syndrome coronavirus 2
SPARMAGNGGDAALALLLLDRLNQLESKMS

SEQ ID NO: 384 - protein, 30 aa, Severe acute respiratory syndrome coronavirus 2
ARMAGNGGDAALALLLLDRLNQLESKMSGK

SEQ ID NO: 385 - protein, 30 aa, Severe acute respiratory syndrome coronavirus 2
RMAGNGGDAALALLLLDRLNQLESKMSGKG

SEQ ID NO: 386 - protein, 30 aa, Severe acute respiratory syndrome coronavirus 2
MAGNGGDAALALLLLDRLNQLESKMSGKGQ

SEQ ID NO: 387 - protein, 30 aa, Severe acute respiratory syndrome coronavirus 2
GNGGDAALALLLLDRLNQLESKMSGKGQQQ

SEQ ID NO: 388 - protein, 30 aa, Severe acute respiratory syndrome coronavirus 2
NGGDAALALLLLDRLNQLESKMSGKGQQQQ

SEQ ID NO: 389 - protein, 30 aa, Severe acute respiratory syndrome coronavirus 2
GGDAALALLLLDRLNQLESKMSGKGQQQQG

SEQ ID NO: 390 - protein, 30 aa, Severe acute respiratory syndrome coronavirus 2
DAALALLLLDRLNQLESKMSGKGQQQQGQT

SEQ ID NO: 391 - protein, 30 aa, Severe acute respiratory syndrome coronavirus 2
LALLLLDRLNQLESKMSGKGQQQQGQTVTK

SEQ ID NO: 392 - protein, 30 aa, Severe acute respiratory syndrome coronavirus 2
LLDRLNQLESKMSGKGQQQQGQTVTKKSAA

SEQ ID NO: 393 - protein, 30 aa, Severe acute respiratory syndrome coronavirus 2
RATRRIRGGDGKMKYLSPRWYFYYLGTGPE

SEQ ID NO: 394 - protein, 30 aa, Severe acute respiratory syndrome coronavirus 2
NAAIVLQLPQGTTLSKGFYAEGSRGGSQAS

SEQ ID NO: 395 - protein, 30 aa, Severe acute respiratory syndrome coronavirus 2
SDAALALLLLDRLNQLESKMSGKGQQQQSQ

SEQ ID NO: 396 - protein, 30 aa, Severe acute respiratory syndrome coronavirus 2
GKMKDLSPRWYFYYLGTGPEAGLLYGANKD

SEQ ID NO: 397 - protein, 30 aa, Severe acute respiratory syndrome coronavirus 2
GTTLPKGFYAEGSRGGSQASSRYSSRSRNS

SEQ ID NO: 398 - protein, 30 aa, Severe acute respiratory syndrome coronavirus 2
DGKMKDLSPRWYFYYLGTGSEAGLPYGANK

SEQ ID NO: 399 - protein, 30 aa, Severe acute respiratory syndrome coronavirus 2
NGGDAALALLLLDRLNQLETKMSGKGQQQQ

SEQ ID NO: 400 - protein, 30 aa, Severe acute respiratory syndrome coronavirus 2
VKQIYKTPPIKDFGGFNFSQILPDPSKPSK

SEQ ID NO: 401 - protein, 30 aa, Severe acute respiratory syndrome coronavirus 2
QMAYRFNGIGVTQNVLYENQKLIANQFNSA

SEQ ID NO: 402 - protein, 30 aa, Severe acute respiratory syndrome coronavirus 2
LYQDVNCTEVPVAIHADQLTPTWRVYSTGS

SEQ ID NO: 403 - protein, 30 aa, Severe acute respiratory syndrome coronavirus 2
YQDVNCTEVPVAIHADQLTPTWRVYSTGSN

SEQ ID NO: 404 - protein, 30 aa, Severe acute respiratory syndrome coronavirus 2
TEVPVAIHADQLTPTWRVYSTGSNVFQTRA

SEQ ID NO: 405 - protein, 30 aa, Severe acute respiratory syndrome coronavirus 2
EVPVAIHADQLTPTWRVYSTGSNVFQTRAG

SEQ ID NO: 406 - protein, 30 aa, Severe acute respiratory syndrome coronavirus 2
FNFSQILPDPSKPSKRSFIEDLLFNKVTLA

SEQ ID NO: 407 - protein, 30 aa, Severe acute respiratory syndrome coronavirus 2
RSFIEDLLFNKVTLADAGFIKQYGDCLGDI

SEQ ID NO: 408 - protein, 30 aa, Severe acute respiratory syndrome coronavirus 2
SFIEDLLFNKVTLADAGFIKQYGDCLGDIA

SEQ ID NO: 409 - protein, 30 aa, Severe acute respiratory syndrome coronavirus 2
GRRGPEQTQGNFGDQELIRQGTDYKHWPQI

SEQ ID NO: 410 - protein, 30 aa, Severe acute respiratory syndrome coronavirus 2
SFIEDLLFNKVTLADVGFIKQYGDCLGDIA

SEQ ID NO: 411 - protein, 30 aa, Severe acute respiratory syndrome coronavirus 2
PNITNLCPFGEVFNATRFTSVYAWNRKRIS

SEQ ID NO: 412 - protein, 30 aa, Severe acute respiratory syndrome coronavirus 2
SFIEDLLFNKVTLADAGFIKQYGDCLGDIV

SEQ ID NO: 413 - protein, 30 aa, Torque teno midi virus
LQKTVQIKNPKKQAPESFLHTWDFRRGFVT

SEQ ID NO: 414 - protein, 30 aa, Torque teno midi virus 11
QQHTKLQLLQLINNLKKKQKLIQLQTGILD

SEQ ID NO: 415 - protein, 30 aa, Torque teno midi virus 12
QIENPERQDPRSILHQWDYRRGFIKERALK

SEQ ID NO: 416 - protein, 30 aa, Torque teno midi virus 7
IQIVNPEKQSPETIIHPWDYRRGLIKEKAL

SEQ ID NO: 417 - protein, 30 aa, Torque teno midi virus 8
KAMLRDWDYRRGIITTTALKRMSEHLQTDS

SEQ ID NO: 418 - protein, 30 aa, Torque teno midi virus 8
ENLQQLILHQQQQQQKLKSNILKLLMDLKH

SEQ ID NO: 419 - protein, 30 aa, Torque teno midi virus 9
SQEIPQTQNLQELIQQQQQQQQQLKYNILK

SEQ ID NO: 420 - protein, 30 aa, Torque teno virus
QLQQQLQFLTREMFKTQAGLHINPMLLNQR

SEQ ID NO: 421 - protein, 30 aa, Severe acute respiratory syndrome coronavirus 2
FKNLREFVFKNIDGYFKIYSKHTPINLVRD

SEQ ID NO: 422 - protein, 30 aa, Severe acute respiratory syndrome coronavirus 2
RTFLLKYNENGTITDAVDCALDPLSETKCT

SEQ ID NO: 423 - protein, 30 aa, Severe acute respiratory syndrome coronavirus 2
PNITNLCPFGEVFNATRFASVYAWNRKRIS

SEQ ID NO: 424 - protein, 30 aa, Severe acute respiratory syndrome coronavirus 2
IRGDEVRQIAPGQTGKIADYNYKLPDDFTG

SEQ ID NO: 425 - protein, 24 aa, Severe acute respiratory syndrome coronavirus 2
LDSKVGGNYNYLYRLFRKSNLKPF

SEQ ID NO: 426 - protein, 30 aa, Severe acute respiratory syndrome coronavirus 2
NGVEGFNCYFPLQSYGFQPTNGVGYQPYRV

SEQ ID NO: 427 - protein, 28 aa, Severe acute respiratory syndrome coronavirus 2
NLVKNKCVNFNFNGLTGTGVLTESNKKF

SEQ ID NO: 428 - protein, 13 aa, Severe acute respiratory syndrome coronavirus 2
LPFQQFGRDIADT

SEQ ID NO: 429 - protein, 29 aa, Severe acute respiratory syndrome coronavirus 2
EVPVAIHADQLTPTWRVYSTGSNVFQTRA

SEQ ID NO: 430 - protein, 22 aa, Severe acute respiratory syndrome coronavirus 2
AGCLIGAEHVNNSYECDIPIGA

SEQ ID NO: 431 - protein, 7 aa, Severe acute respiratory syndrome coronavirus 2
VKQIYKT

SEQ ID NO: 432 - protein, 6 aa, Severe acute respiratory syndrome coronavirus 2
EDLLFN

SEQ ID NO: 433 - protein, 30 aa, Severe acute respiratory syndrome coronavirus 2; identical to SEQ ID NO: 401

SEQ ID NO: 434 - protein, 30 aa, Severe acute respiratory syndrome coronavirus 2
SSTASALGKLQDVVNQNAQALNTLVKQLSS

SEQ ID NO: 435 - protein, 7 aa, Severe acute respiratory syndrome coronavirus 2
EELDKYF

SEQ ID NO: 436 - protein, 30 aa, Severe acute respiratory syndrome coronavirus 2
YFKNHTSPDVDLGDISGINASVVNIQKEID

SEQ ID NO: 437 - protein, 12 aa, Severe acute respiratory syndrome coronavirus 2
DRLNEVAKNLNE

SEQ ID NO: 438 - protein, 6 aa, Severe acute respiratory syndrome coronavirus 2
GSNQNG

SEQ ID NO: 439 - protein, 29 aa, Severe acute respiratory syndrome coronavirus 2
GKMKDLSPRWYFYYLGTGPEAGLPYGANK

SEQ ID NO: 440 - protein, 20 aa, Severe acute respiratory syndrome coronavirus 2
VATEGALNTPKDHIGTRNPA

SEQ ID NO: 441 - protein, 4 aa, Severe acute respiratory syndrome coronavirus 2
TLPK

SEQ ID NO: 442 - protein, 7 aa, Severe acute respiratory syndrome coronavirus 2
LLDRLNQ

SEQ ID NO: 443 - protein, 30 aa, Severe acute respiratory syndrome coronavirus 2
TVTKKSAAEASKKPRQKRTATKAYNVTQAF

SEQ ID NO: 444 - protein, 21 aa, Severe acute respiratory syndrome coronavirus 2
QTQGNFGDQELIRQGTDYKHW

SEQ ID NO: 445 - protein, 16 aa, Severe acute respiratory syndrome coronavirus 2
FKDQVILLNKHIDAYK

SEQ ID NO: 446 - protein, 8 aa, Severe acute respiratory syndrome coronavirus 2
PKKDKKKK

SEQ ID NO: 447 - protein, 13 aa, Severe acute respiratory syndrome coronavirus 2
QTVTLLPAADLDD

SEQ ID NO: 448 - protein, 10 aa, Severe acute respiratory syndrome coronavirus 2
EELDKYFKNH

SEQ ID NO: 449 - protein, 63 aa, Severe acute respiratory syndrome coronavirus 2
VKQIYKTPPIKDFGGFNFSQILPDPSKPSKRFIEDLLFNKVTLADAGFIKQYGDCLGDIAARD

SEQ ID NO: 450 - protein, 63 aa, Severe acute respiratory syndrome coronavirus 2
VKQMYKTPTLKYFGGFNFSQILPDPLKPTKRFIEDLLFNKVTLADAGFMKQYGECLGDINARD

SEQ ID NO: 451 - protein, 70 aa, Severe acute respiratory syndrome coronavirus 2
VKSSQSSPIIPGFGGDFNLTLLEPVSISTGSARSIESDALLFDKVTIAIDPGYMQGYDDCMQQGPASARD

SEQ ID NO: 452 - protein, 67 aa, Severe acute respiratory syndrome coronavirus 2
LMNGVTLSTKGLKVNFNVDDINFSPVLGCLGSECASIKEADSLSLFDKVKLSDVGNFNCTGGAEIRD

SEQ ID NO: 453 - protein, 69 aa, Severe acute respiratory syndrome coronavirus 2
LMQGVTLSSNLNTNLHSDVDNIDFKSLLGCLGSQLCLGEDSLSSLFNVKLSDVGFVEAYNNTGGSEIRD

SEQ ID NO: 454 - protein, 66 aa, Severe acute respiratory syndrome coronavirus 2
DKKAFTLANVSSGDYNLSSVIPSLPTSGSRVAGAIEDILFSKLVTSGLGTVDADYKSCTKGLSIAD

SEQ ID NO: 455 - protein, 68 aa, Severe acute respiratory syndrome coronavirus 2
DSNAFSLANVTSFGDYNLSSVLPQRINRISSRIAGALEDLLFSKVVTSGLGTVDVDYKSCTKGLSIAD

SEQ ID NO: 456 - protein, 57 aa, Severe acute respiratory syndrome coronavirus 2
DVVIGIVNNTVYDPLQPEDSFKEELDKYFKNHTSPDVDLGDISGINASVVNIQKEID

SEQ ID NO: 457 - protein, 55 aa, Severe acute respiratory syndrome coronavirus 2
DVVIGINNTVYDPPELDSFKEELDKYFKNHTSPDVDLGDISGINASVVNIQKEID

SEQ ID NO: 458 - protein, 60 aa, Severe acute respiratory syndrome coronavirus 2
QVTYQNISTNLPPPLLGNSTGIDFQDELDEFFKNVSTSIPNFGSLTQINTTLLDLTYEML

SEQ ID NO: 459 - protein, 56 aa, Severe acute respiratory syndrome coronavirus 2
AVNYTKAPYVMLNTSIPNLPDFKEELDQWFKNQTSVAPDSLDYINVTFLDLQVEMN

SEQ ID NO: 460 - protein, 56 aa, Severe acute respiratory syndrome coronavirus 2
SVNTKAPFIYLNNSIPNLSDFAELSLWFKNHTSIAPNLTFNSHINATFLDLYYEMN

SEQ ID NO: 461 - protein, 63 aa, Severe acute respiratory syndrome coronavirus 2
NVTFVNISRELQTIVPEYIDVNKTLQELSYKLPNYTVPDLVVEQYNQILNLTSEISTLENKSA

SEQ ID NO: 462 - protein, 66 aa, Severe acute respiratory syndrome coronavirus 2
NVTFVNISRVELHTVIPDYVDVNKTLQEFAQNLPKYVKPNFDLTPFNLTYLNLSEEKLKQLEAKTA

SEQ ID NO: 463 - protein, 30 aa, Severe acute respiratory syndrome coronavirus 2
APYVMLNTSIPNLPDFKEELDQWFKNQTSV

SEQ ID NO: 464 - protein, 23 aa, Severe acute respiratory syndrome coronavirus 2
SQSIIAYTMSLGAENSVAYSNNS